SEQUENCE LISTING (2 sequences)

SEQ ID NO: 1 - DNA, 21 bp, Artificial Sequence
  Forward primer (A3243+)
ccaagaacagggtttgttaag

SEQ ID NO: 2 - DNA, 22 bp, Artificial Sequence
  Reverse primer (A3243G-)
tgttaagaagaggaattgaacc

(19) United States
(12) Patent Application Publication
(10) Pub. No.: US 2023/0043700 A1
(43) Pub. Date: Feb. 9, 2023

(54) ISOLATED MITOCHONDRIA WITH SMALLER SIZE AND LIPID MEMBRANE-BASED VESICLES ENCAPSULATING ISOLATED MITOCHONDRIA

(71) Applicant: LUCA SCIENCE INC., Tokyo (JP)

(72) Inventors: Masashi SUGANUMA, Tokyo (JP); Yuma YAMADA, Hokkaido (JP); Hideyoshi HARASHIMA, Hokkaido (JP); Daisuke SASAKI, Hokkaido (JP); Mitsue HIBINO, Hokkaido (JP); Yoshihiro OHTA, Tokyo (JP)

(21) Appl. No.: 17/789,401

(22) PCT Filed: Dec. 25, 2020
(86) PCT No.: PCT/JP2020/049314

§ 371 Date: Jun. 27, 2022

(30) Foreign Application Priority Data
Dec. 27, 2019 (JP) 2019-239479

(51) Int. Cl. A61K 9/127; A61K 35/12; C12Q 1/6883; A61P 43/00

(52) U.S. Cl. CPC A61K 9/1272; A61K 35/12; A61K 9/1277; C12Q 1/6883; A61P 43/00; C12Q 2600/158

(57) ABSTRACT

According to the present invention, there is provided a composition comprising a population of mitochondria with a smaller size and a population of lipid membrane-based vesicles encapsulating mitochondria in a closed space and a method for producing the composition.

### CROSS REFERENCE TO RELATED APPLICATIONS

This application claims the benefit of priority to Japan application number 2019-239479, filed on Dec. 27, 2019, the entire contents of which are hereby incorporated by reference.

### TECHNICAL FIELD

The present invention relates to isolated mitochondria with a smaller size and lipid membrane based vesicles encapsulating isolated mitochondria.

### BACKGROUND

Mitochondrial dysfunction, for example, respiratory chain complex dysfunction is a major cause responsible for a mitochondrial disease and aging. Decreased mitochondrial function influences cells in many organs principally involved in mitochondrial diseases and age-related diseases. To overcome this, attempts to introduce mitochondria into cells have been made (U.S. Pat. No. 9,603,872B). In Patent Literature 1, mitochondria are mixed with lipofectamine 2000 reagent to obtain lipoplexes of mitochondria with lipofectamine (i.e., associations of lipofectamine particles and free-form mitochondrial particles). In U.S. Pat. No. 9,603,872B, it was confirmed that the lipoplexes obtained were introduced into cells. However, in U.S. Pat. No. 9,603,872B, physiological actions of the introduced lipoplexes have not yet been confirmed.

A technology called a micro flow channel device has been developed for forming liposomes by using a channel having a width of 100 to 200 μm (Kimura N. et al., ACS Omega, 3: 5044-5051, 2018). According to Kimura N. et al., 2018, vesicles of lipid-membrane base and having a particle size of about 10 nm to 100 nm can be obtained.

### BRIEF SUMMARY

The present invention provides lipid membrane-based vesicles encapsulating isolated mitochondria and a method for producing the vesicles.

The present inventors obtained a composition comprising a population of lipid membrane-based vesicles encapsulating mitochondria. The vesicles each conceivably have a bag-like membrane structure (closed space) formed of lipid membrane for comprising mitochondria.

Herein, for example, the following inventions are provided.

(1) A composition comprising a population of lipid membrane-based vesicles encapsulating mitochondria.

(2) The composition according to item (1), wherein the population of lipid membrane-based vesicles has a particle size distribution, as determined by dynamic light scattering, having a peak at less than 1 μm.

(3) The composition according to item (2), wherein the population of lipid membrane-based vesicles has a particle size distribution, as determined by dynamic light scattering, having a peak at less than 500 nm.

(4) The composition according to any one of items (1) to (3), wherein the population of lipid membrane-based vesicles has a PDI of less than 0.5.

(5) The composition according to any one of items (1) to (4), wherein the encapsulated mitochondria can be incorporated into the cytoplasm of the cells in contact therewith and the mitochondria can be fused with endogenous mitochondria in the cytoplasm.

(6) The composition according to any one of items (1) to (6), for use in delivering mitochondria into cells.

(7) The composition according to item (6), for use in improving respiratory activity of mitochondria in cells.

(8) A method for producing the composition according to item (1), comprising:

bringing an aqueous solution comprising isolated mitochondria with an ethanol solution comprising a lipid that can form lipid membrane into contact with each other in a confluent channel within a micro flow channel device to mix the solutions.

(9) The method according to item (8), wherein the micro flow channel device comprises a flow channel for facilitating mixing of the solutions brought into contact with each other in the confluent channel, the flow channel having a baffle construct.

Also, the following inventions are provided.

[1] A composition comprising a population of mitochondria, wherein the population has a particle size distribution, as determined by dynamic light scattering, having a peak at less than 1 μm.
[2] The composition according to item [1], wherein the population has a particle size distribution, as determined by dynamic light scattering, having a peak at less than 500 nm.
[3] The composition according to item [1] or [2], wherein the population has a PDI of less than 0.5.
[4] A composition comprising a population of lipid membrane-based vesicles encapsulating mitochondria, wherein the population of lipid membrane-based vesicles has a particle size distribution, as determined by dynamic light scattering, having a peak at less than 1 μm.
[4A] The composition according to item [1], wherein 50% or more of the mitochondria are each encapsulated in a lipid membrane-based vesicle.
[5] The composition according to item [4] or [4A], wherein the population of lipid membrane-based vesicles has a particle size distribution, as determined by dynamic light scattering, having a peak at less than 500 nm.
[6] The composition according to any one of items [4] to [5] (i.e., items [4], [4A] and [5]), wherein the population of lipid membrane-based vesicles has a PDI of less than 0.5.
[7] The composition according to any one of items [4] to [6], wherein the encapsulated mitochondria can be incorporated into the cytoplasm of cells in contact therewith, and the mitochondria can be fused with endogenous mitochondria in the cytoplasm.
[8] The composition according to any one of items [4] to [7], for use in delivering mitochondria into cells.
[9] The composition according to item [8], for use in improving respiratory activity of mitochondria in cells.
[10] A method for producing the composition according to item [4] or [4A], comprising bringing an aqueous solution comprising isolated mitochondria and an ethanol solution comprising a lipid that can form lipid membrane into contact with each other in a confluent channel within a micro flow channel device to mix the solutions.
[11] The method according to item [10], wherein the micro flow-channel device comprises a flow channel for facilitating mixing of the solutions brought into contact with each other in the confluent channel, the flow channel having a baffle construct.

Mitochondria can be encapsulated in vesicles in the present invention, which is suitable for a pharmaceutical formulation. A population of lipid membrane-based nano vesicles containing mitochondria having a monodispersed size distribution (i.e., PDI≤0.5) and/or a peak at less than 1 μm in the size distribution will be further suitable for a pharmaceutical formulation.

[12] A method of measuring mitochondria DNA level in the isolated mitochondria or the encapsulated mitochondria, comprising amplifying at least a part of the mitochondria DNA in a sample including the isolated mitochondria or the encapsulated mitochondria to obtain amplicon of the amplified DNA and counting the amplicon to obtain the mitochondria DNA level.
[13] The method according to item [12], further comprising comparing the measured mitochondria DNA level to a standard value.

### BRIEF DESCRIPTION OF THE DRAWINGS

FIG. 1 shows the particle size distribution and PDI, as determined by dynamic light scattering (DLS) and zeta potential (ζ) on a suspension of mitochondria isolated from cells. a) shows data of isolated mitochondria (product to be prepared at time of use) before freezing and b) shows data of isolated mitochondria (frozen product) after a freeze-and-thaw process.

FIG. 2 shows the results of polarization of mitochondria in a product to be prepared at time of use and a frozen product detected with a fluorescent dye (TMRE). Fluorescence was observed from each of the product to be prepared at time of use and the frozen product.

FIG. 3, panel a) shows a scheme of preparation of nano vesicles using a micro flow channel device and subsequent dialysis. FIG. 3, panel b) shows the scheme of preparing nanoparticles by bringing a lipid-containing organic phase and an aqueous buffer (aqueous phase) into contact with each other in a confluent channel within a micro flow channel device to mix them; compositions of the organic phase and aqueous phase and flow rates thereof, the particle size distribution by DLS of the obtained nanoparticles, PDI and zeta potential (ζ) thereof.

FIG. 4 shows the scheme of preparing lipid membrane-based vesicles encapsulating mitochondria by bringing a lipid-containing organic phase and isolated mitochondria-containing aqueous buffer (aqueous phase) into contact with each other in a confluent channel within a micro flow channel device to mix them; compositions of the organic phase and aqueous phase and flow rates thereof, the particle size distribution, and PDI by DLS of the obtained vesicle and zeta potential (ζ) thereof.

FIG. 5-1, panel a) shows the particle size distribution and PDI by DLS of the particles obtained and zeta potential (ζ) thereof in the case where the organic phase is an organic phase containing no lipid (50% ethanol solution) and the aqueous phase is an aqueous buffer containing isolated mitochondria; and panel b) shows the particle size distribution and PDI by DLS of the particles obtained in the case where the organic phase is an organic phase containing stearylated octaarginine (STR-R8) and the aqueous phase is an aqueous buffer containing isolated mitochondria and zeta potential (ζ) thereof.

FIG. 5-2, panel c) shows the particle size distribution and PDI by DLS of the particles obtained in the case where an aqueous buffer was used in place of the organic phase and the aqueous phase is an aqueous buffer containing isolated mitochondria, and zeta potential (t) thereof.

FIG. 6 shows fluorescence microscopic images of nanoparticles prepared by a micro flow channel device in accordance with the scheme of FIG. 4 (herein, lipid is fluorescently stained with DOPE-N-(7-nitro-2-1,3-benzoxadiazol-4-yl)(NBD-DOPE); while mitochondria are stained with MitoTracker (trademark) Deep Red). Panel a) shows fluorescent signals from isolated mitochondria; panel b) shows fluorescent signals from lipids, and panel c) shows merged signals of these, indicating that they are almost completely co-localized.

FIG. 7 shows an electron microscopic image of an isolated mitochondrion fixed by a chemical fixation method.

FIG. 8 shows an electron microscopic image of a nano-capsule prepared in accordance with the scheme of FIG. 3, panel b) and stained by a negative staining method. FIG. 8 shows that the inside of the nano-capsule obtained is filled up with lipid (lipid layer) against expectation.

FIG. 9 shows an electron microscopic image of a nanoparticle prepared in accordance with the scheme of FIG. 4 and stained by a negative staining method. In the negative staining method, mitochondria did not create contrast, with the result that mitochondria themselves cannot be detected.

FIG. 10 shows an electron microscopic image of isolated mitochondria obtained in accordance with the scheme of FIG. 5-1, panel b), treated with STR-R8 and fixed by a chemical fixation method.

FIG. 11 shows the particle size distributions and PDI by DLS of mitochondria-encapsulating lipid membrane-based vesicles obtained in accordance with the same scheme as in FIG. 4 except that the types of lipids were changed, and zeta potential (ζ) thereof. Panels a) and b) show the data of negative controls of using the aqueous solution containing no mitochondria. Panels c) and d) show the results obtained in the same conditions respectively as in panels a) and b) except that the aqueous solution is an aqueous buffer containing isolated mitochondria.

FIG. 12 shows the flow rate of the solution to be introduced into a micro flow channel device; the particle size distribution and PDI by DLS of mitochondria-encapsulating lipid membrane-based vesicles obtained; and zeta potential (ζ).

FIG. 13 shows confocal laser scanning microscopic images of cells obtained by bringing the mitochondria-encapsulating lipid membrane-based vesicles obtained into contact with human cultured cells and incubating them for 3 hours. In FIG. 13, mitochondria present within human cells are stained with MitoTracker (trademark) Green; and the mitochondria in the mitochondria-encapsulating lipid membrane-based vesicles are stained with MitoTracker (trademark) Deep Red. In the cells incubated, the images formed of fluorescence respectively emitted from these mitochondria almost completely match in localization (see, HeLa mito green, isolated mito red, and Merge).

FIG. 14 shows confocal laser scanning microscopic images of cells obtained by bringing isolated mitochondria not packaged in vesicles into contact with human cultured cells and incubating them for 3 hours. In FIG. 14, mitochondria present within the human cells were stained with MitoTracker (trademark) Green; and the isolated mitochondria were stained with MitoTracker (trademark) Deep Red. FIG. 14 shows that signals derived from isolated mitochondria were virtually not observed within the cells.

FIG. 15 shows confocal laser scanning microscopic images of cells obtained by bringing isolated mitochondria not packaged in vesicles and treated with STR-R8 into contact with human cultured cells and incubating them for 3 hours. In FIG. 15, mitochondria present within human cells were stained with MitoTracker (trademark) Green; and the isolated mitochondria were stained with MitoTracker (trademark) Deep Red. FIG. 15 shows that signals derived from isolated mitochondria were virtually not observed within the cells. In FIG. 15, the left bottom panel shows optical microscopic image of human cultured cells incubated, suggesting that cell death is induced.

FIG. 16 shows confocal laser scanning microscopic images of cells obtained by bringing isolated mitochondria not packaged in vesicles into contact with human cultured cells (human cardiac precursor cells) and incubating them for 3 hours. In FIG. 16, mitochondria present within human cells were stained with MitoTracker (trademark) Green; and the isolated mitochondria were stained with MitoTracker (trademark) Deep Red. FIG. 16 shows that signals derived from isolated mitochondria were not virtually observed within the cells.

FIG. 17 shows confocal laser scanning microscopic images of cells obtained by bringing isolated mitochondria not packaged in vesicles and treated with STR-R8 into contact with human cultured cells (human cardiac precursor cells) and incubating them for 3 hours. In FIG. 17, mitochondria present within human cells were stained with MitoTracker (trademark) Green; whereas, the isolated mitochondria were stained with MitoTracker (trademark) Deep Red. FIG. 17 shows that signals derived from isolated mitochondria were virtually not observed within the cells.

FIG. 18 shows the scheme of obtaining lipid membrane-based vesicles encapsulating hCDC-derived mitochondria by obtaining human cardiac muscle stem cells (hCDC) from the cardiac muscle and isolating mitochondria from hCDC.

FIG. 19 shows the measurement results of mitochondrial respiratory activity of cells obtained by bringing lipid membrane-based vesicles encapsulating hCDC-derived mitochondria into contact with skin fibroblasts obtained from a MELAS patient and incubating them for 3 hours or 24 hours.

FIG. 20 shows the measurement results of mitochondrial respiratory activity of cells obtained by bringing lipid membrane-based vesicles encapsulating hCDC-derived mitochondria into contact with skin fibroblasts obtained from a LHON patient and incubating them for 3 hours or 24 hours.

FIG. 21 shows the measurement results of mitochondrial respiratory activity of cells obtained by bringing lipid membrane-based vesicles encapsulating hCDC-derived mitochondria or a lipid complex of lipofectamine and mitochondria (LFN iso Mt), into contact with normal fibroblasts, and incubating them for 24 hours. LFN iso Mt reduces mitochondrial respiratory activity of the cells. From this, it was considered that LFN iso Mt enters into the cells and causes toxicity against mitochondria.

FIG. 22 shows the measurement results of mitochondrial respiratory activity of cells obtained by bringing lipid membrane-based vesicles encapsulating hCDC-derived mitochondria or a lipid complex of lipofectamine and mitochondria (LFN iso Mt), into contact with skin fibroblasts obtained from a Leigh encephalopathy patient, and incubating them for 24 hours.

FIG. 23 shows the measurement results of mitochondrial respiratory activity of cells obtained by bringing lipid membrane-based vesicles encapsulating hCDC-derived mitochondria or a lipid complex of lipofectamine and mitochondria (LFN iso Mt), into contact with skin fibroblasts obtained from a LHON patient, and incubating them for 24 hours.

FIG. 24 shows the capability of mitochondria-encapsulating lipid membrane-based vesicles and LFN iso Mt to be incorporated into cells.

FIG. 25 shows the particle size distribution and PDI by DLS of LFN iso Mt, and zeta potential (ζ). In LFN iso Mt, the zeta potential value is close to 0. From this, it was considered that a complex in which negatively charged mitochondria and positively charged LFN are electrically neutralized is obtained.

FIG. 26 shows electron microscopic images of lipofectamine 2000 (LFN) and a mixture of LFN and isolated mitochondria (LFN+Mt), negatively stained (in panels B and A, respectively).

FIG. 27 shows electron microscopic images of isolated mitochondria (Panel A) and a mixture of lipofectamine 2000 (LFN) and isolated mitochondria (LFN+Mt), stained by chemical fixation (in Panel B). FIG. 27, Panel C shows the survival ratio of the cells treated with MITO-Q and LFN+Mt, respectively.

FIG. 28 shows a schematic figure of a complex of lipofectamine and isolated mitochondria (panel a) and a schematic figure of lipid membrane-based vesicles encapsulating mitochondria (panel b) based on the obtained results.

FIG. 29A shows the result of the assay for the membrane potential of the mitochondria isolated by various methods indicated in the figure. MitoTracker Deep Red was used as a mitochondrial membrane potential indicator.

FIG. 29B shows the result of the assay of the membrane potential of the mitochondria isolated by various methods indicated in the figure. Tetramethylrhodamine methyl ester (TMRM) was used as a mitochondrial membrane potential indicator.

FIG. 30A shows the overview of the assay for the mitochondrial respiration activity in the cells treated with the encapsulated mitochondria or the LFN-treated mitochondria.

FIG. 30B shows the results of the assay for the mitochondrial respiration activity.

FIG. 30C shows the results of the assay for the mitochondrial respiration activity in a bar graph format.

FIG. 31A shows the calibration curve fitted for the DNA concentration by quantitative PCR and the protein concentration measured by BradFord method.

FIG. 31B shows the calibration curve fitted for the copy number of the amplicon by PCR method and the template concentration.

FIG. 31C shows the copy number of mtDNA in the mitochondria isolated by the methods indicated in the figure. The copy number was standardized using total mitochondrial protein amount.

FIG. 31D shows the copy number of mtDNA in the encapsulated mitochondria which has been isolated by the methods indicated in the figure. The copy number was standardized using total mitochondrial protein amount.

FIG. 31E shows the basal respiration activity of the cells treated with the encapsulated mitochondria which has been isolated by the methods indicated in the figure.

FIG. 31F shows the maximal respiration activity of the cells treated with the encapsulated mitochondria which has been isolated by the methods indicated in the figure.

FIG. 31G shows the amount of TFAM in the mitochondria isolated by the methods indicated in the figure. The amount was standardized using total mitochondrial protein amount.

FIG. 31H shows the concentration of the total protein included in the mitochondria isolated by the various methods indicated in the figure. D-Mt was isolated by the conventional detergent method, Q (pH 7.4) was isolated by iMIT using pH 7.4 buffers, and Q (pH 8.9) was isolated by iMIT using pH 8.9 buffers.

FIG. 32A shows the size distribution of the hCPC-MITO-Q prepared by encapsulating the mitochondria isolated by iMIT method from human cardiac progenitor cells (hCPC).

FIG. 32B shows the results of the staining of the mitochondria inside the cell treated with or without hCPC-MITO-Q with TMRM.

FIG. 33 shows the results of the mitochondrial respiration activity in the cells treated with the samples indicated in the figure. The term “Res-hCPC-MITO-Q” represents the MITO-Q prepared from the hCPCs treated with MITO-Porter encapsulating resveratrol.

FIG. 34A shows the time course of the assay protocol.

FIG. 34B shows the maximal respiration activity of the cells treated with MITO-Q after the incubation time indicated in the figure.

FIG. 35 shows the maximal respiration activity of the cells treated with MITO-Q that has been stored at 4° C. for various periods indicated in the figure.

FIG. 36 shows a reference figure showing the structure of a micro flow channel device. In the figure, a big arrow indicates the flow direction of liquid of the channel.

### DETAILED DESCRIPTION

In the specification, the “mitochondria” is an intracellular organelle present in the cytoplasm of eukaryotic cells. The mitochondria conceivably play a role in producing ATP (by oxidative phosphorylation) within a cell through the electron transport system. Mitochondria have own DNA (mitochondrial DNA), which encodes mitochondrial constituent factors (for example, proteins of the respiratory chain complex in the electron transport system), which is independent of DNA of cellular nucleus. A mutation of mitochondrial DNA sometimes impairs the mitochondrial functions. Malfunction of the mitochondria may cause a disease called as a mitochondrial disease. To overcome this, an attempt to supply exogeneous mitochondria has been carried out as a therapy.

In the specification, the “vesicle” refers to an object in a particle form having a closed space surrounded by membrane, inside. The closed space refers to a space to/from which physical, chemical and/or physiological migration of substances is restricted.

In the specification, the “lipid membrane-based vesicle” refers to a vesicle, such as a liposome, formed of membrane comprising a lipid as a main constituent. Amphipathic lipid, or cationic or anionic lipid, which is placed in an aqueous solution, can form vesicles, which are composed of a lipid bilayer (particularly, a single lipid bilayer) having a closed space comprising an aqueous solution therein.

In the specification, the “encapsulating” refers to the state where a predetermined substance is encapsulated within a closed space to/from which migration of substances is restricted. Therefore, an encapsulated mitochondrion will be separated into a closed space in a hollow sac or vesicle formed by membrane structure. A lipid membrane-based vesicle having a lipid bilayer membrane usually is called a liposome. Lipid membrane structure usually has no or little permeability against water, blood, or other aqueous solution. An encapsulated mitochondrion can therefore be protected by the membrane structure, which fully packages the whole mitochondrion, in a body fluid during a delivery of mitochondria to a cell. Membrane structure can facilitate the encapsulated mitochondrion into a cell when the composition of the membrane is similar to that of a cell membrane or when the surface of the membrane structure has a positive charge. Whether mitochondria are encapsulated in a vesicle of lipid-membrane base or not can be confirmed, for example, by separately staining mitochondria and lipid, forming a vesicle encapsulating the mitochondria therewith and finding co-presence of respective pigments of the mitochondria and the lipid, when observed by an optical microscope (e.g., by a fluorescence microscope if a fluorescent dye is used) and finding the presence of a hollow lipid membrane (or its cross section) by negative staining when morphology is observed by an electron microscope. Whether mitochondria are encapsulated in a vesicle of lipid-membrane base or not can be confirmed, for example, by bringing the vesicle into contact with cells and observing whether the mitochondria can be introduced into the cytoplasm; or may be confirmed by, if the lipid membrane of a vesicle is positively charged, checking whether a positive zeta potential of, for example, 10 mV or more, is obtained or not. In the specification, the “free-form” mitochondria refer to isolated mitochondria and particularly used in clearly describing mitochondria not encapsulated in a vesicle. In the specification, unless otherwise specified, “isolated mitochondria” or “mitochondria” refer to free-form mitochondria.

In the specification, the “population” refers to a group of a plurality of the same or different substances. In the specification, the “population of lipid membrane-based vesicles encapsulating mitochondria” is a group of at least a plurality of the same or different mitochondria-encapsulating lipid membrane-based vesicles. The population may not be always homogenous and may have physical, chemical and/or physiological distributions. The physical distribution includes, for example, particle size and polydispersity index. The chemical distribution includes, for example, a zeta potential distribution and a lipid composition distribution. The physiological distribution include, for example, a difference of physiological function (for example, respiratory activity).

In the specification, the “dynamic light scattering” (DLS) refers to a technique for determining the sizes of fine particles in the nanometer order in a solution. The methods for measuring particle sizes and polydispersity are defined, for example, in ISO22412: 2017. In the dynamic light scattering, the size distribution of particles can be obtained. Specifically, an average particle size can be obtained from an autocorrelation function of scattering light intensity in accordance with the Cumulant analysis method (ISO22412). In the specification, the “peak” refers to a portion indicating a maximum frequency in the histogram showing the particle size distribution measured. If mitochondria are isolated intact, the isolated mitochondria conceivably show a particle size distribution having a peak usually around at about 1 μm.

In the specification, “polydispersity index” (PDI) (or referred to as polydispersity) is an index of evaluating the breadth of the particle size distribution obtained by DLS. PDI can be obtained from an autocorrelation function of scattering light intensity in accordance with the Cumulant analysis method (ISO22412). PDI=0 means that a group of particles in a solution consists of completely the same-size particles, and PDI is up to 1. If PDI is 0.5 or more, a group of particles is considered to have poly-dispersity. The particle size distribution of mitochondria (intracellular organelle) has polydispersity and isolated mitochondria are considered to usually have a PDI of 0.5 or more.

In the specification, the “zeta potential” (ζ potential) refers to the potential which can be calculated by electrophoretic light scattering in accordance with the Helmholtz-Smoluchowski equation. The zeta potential is defined as follows. When particles move relative to a solution, a layer of the solution having a certain thickness moves together with the particles. The potential difference between the surface of the layer (slipping plane) and the bulk portion of the solution, which is positioned sufficiently distant from the surface, is defined as the zeta potential. The zeta potential can be measured by an electrophoretic light scattering method and obtained in accordance with the Helmholtz-Smoluchowski equation based on the dielectric constant of the solution, viscosity of the solution, the migration speed of particles and the electric field. Mitochondria release protons from the inner membrane to the outside during respiration. Due to this, mitochondria are negatively polarized and have a minus zeta potential. When mitochondria are encapsulated in a vesicle of lipid-membrane base, the zeta potential is determined based on the composition of the lipid-membrane base. Thus, the effect of mitochondria on the zeta potential is none or limited.

In the specification, the “respiration” refers to an activity of mitochondria to produce ATP by consuming oxygen by use of the concentration gradient of protons released by mitochondria from the interior in the electron transport system. The respiratory activity of mitochondria refers to respiration capacity of mitochondria, which can be determined, for example, by oxygen consumption rate (OCR) of mitochondria. The oxygen consumption rate can be determined, for example, by an extracellular flux analyzer. To describe more specifically, a substrate of the respiratory chain complex, such as malic acid, is added to mitochondria, and then, the OCR of a solution of the mitochondria (designated as “OCR1”) is measured. Thereafter, an ATP synthetase inhibitor (for example, oligomycin) is added to mitochondria, and then, OCR of a solution of the mitochondria (designated as “OCR2”) can be measured. Subsequently, an uncoupler (for example, FCCP) is added to mitochondria, and then, OCR of a solution of the mitochondria (designated as “OCR3”) can be measured. After that, a respiratory chain complex inhibitor (for example, an inhibitor of complex I such as rotenone and an inhibitor of complex III such as antimycin A) is added, and then, OCR of a solution of the mitochondria (designated as “OCR4”) can be measured. The basic respiration rate, ATP production respiration rate and maximum respiration rate of mitochondria can be obtained in accordance with the following equations.

Basic respiration rate of mitochondria=OCR1−OCR4;

ATP production respiration rate of mitochondria=OCR1−OCR2;

Maximum respiration rate of mitochondria=OCR3−OCR4.

In an embodiment, the respiratory activity of mitochondria to be assayed can be the maximum respiratory activity of mitochondria.

In the specification, the “micro flow channel device” or “micro fluidic device” is used interchangeably, and refers to a device comprising a channel, which has a diameter or a width and height in the order of μm. In the channel of the micro flow channel device, two different compositions introduced from two different inlets can be joined in a confluent channel. The confluent channel is a portion at which two flow paths respectively connecting to two different inlets, are joined. The micro flow channel device may have a channel for mixing the solutions (mixing channel) joined besides the confluent channel. The mixing channel may have a structure (for example, bend) for facilitating mixing and stirring. The mixing channel also may or may not have concave and convex in the inner surface.

In the specification, the “ethanol solution” refers to an aqueous solution comprising ethanol. In the specification, the “organic phase” refers to a phase comprising an organic solvent, and may be a phase comprising an organic solvent that can dissolve a lipid that can form lipid membrane. The organic solvent in the organic phase can be, for example, an organic solvent soluble in water. The water soluble organic solvent is advantageous because it is easily removed by a method such as dialysis after formation of vesicles. As an example of the water soluble organic solvent, ethanol may be mentioned. In the embodiment, the organic phase may be, for example, an aqueous ethanol solution.

The present inventors found that lipid membrane-based vesicles encapsulating mitochondria and a population of the vesicles can be obtained by bringing an aqueous solution comprising isolated mitochondria and an organic phase (for example, ethanol solution) comprising a lipid that can form lipid membrane, into contact with each other in the confluent channel of a micro flow channel device to mix them. According to the present invention, there is provided a method for producing lipid membrane-based vesicles encapsulating mitochondria or a population thereof or a composition comprising the vesicles, comprising bringing an aqueous solution comprising isolated mitochondria and an organic phase (for example, ethanol solution) comprising a lipid that can form lipid membrane into contact with each other in the confluent channel within a micro flow channel device to mix them.

As used herein, the term “mitochondria activating agent” means a substance capable of activating a mitochondrial respiratory chain complex (electron transport system), particularly a substance capable of bringing mitochondria into a polarized state in terms of a membrane potential, and in particular, it is preferable to use a substance capable of bringing mitochondria into a hyperpolarized state. Examples of the mitochondria activating agent may include antioxidants such as resveratrol (3,5,4′-trihydroxy-trans-stilbene), coenzyme Q10, vitamin C, vitamin E, N-acetylcysteine, 2,2,6,6,-tetramethylpiperidine 1-oxyl (TEMPO), superoxide dismutase (SOD) and glutathione, and in particular, resveratrol is preferable (see WO2018/092839). The resveratrol that is preferably used in the present invention may be one extracted from a plant by a known method, or one chemically synthesized by a known method such as, for example, Andrus et al.'s method (Tetrahedron Lett. 2003, 44, pp. 4819-4822). Other Examples of mitochondria activating agent include mitochondria DNA, and mitochondria RNA such as 12S rRNA and 16S rRNA (see WO2020/230601, which is incorporated herein by reference in its entirety), and any other component of mitochondria.

The micro flow channel device comprises a confluent channel at which flow channels extending from at least two inlets are joined. The micro flow channel device may further comprise a mixing channel for facilitating mixing of the solutions joined at the confluent channel. The mixing channel may be a linearly extending path or may have at least one bend in order to further facilitate mixing (for example, a baffle structure or a plurality of bends continuously arranged). The mixing channel may or may not have concave and convex in the inner surface.

The channel of the micro flow channel device may have a thickness in the order of μm. The thickness of the channel if the channel has a circular cross section is represented by the diameter thereof. If the cross section is an ellipse, the thickness may be represented by either one of the major axis and minor axis or both of them. If the cross section is a rectangle, either one of the width and height or both of them may be employed. The width and height of the channel each independently can be, for example, 100 μm to 400 μm. Referring to FIG. 36, the micro flow channel device that can be suitably used in the present invention will be described. As shown in FIG. 36, the channel (10) of the micro flow channel device has two liquid-sample inlets (11a and 12a), channels (11 and 12) each connecting between the liquid-sample inlets (11a and 12a) and a confluent channel (13), and a mixing channel (14). The confluent channel 13 is a site at which the channels (11 and 12) extending respectively from the two liquid-sample inlets are joined. The mixing channel 14 is a channel for mixing the liquid samples joined. The mixing channel 14 may be linear channel or a channel having bends. As shown in FIG. 36, in the mixing channel 14, the solution moves along with the flow direction indicated by a big arrow and is guided toward an outlet 14c via bends. The mixing channel 14 may have a single or a plurality of sets of bends represented by 14a and 14b (for example, 10 to 30 sets, 15 to 25 sets, 20 sets). In FIG. 36, 14a represents the region at which the channel is narrowed in width and 14b represents the region at which the narrowed channel is widened.

Examples of the lipid that can form lipid membrane in a lipid membrane-based vesicle include a phospholipid, a glycolipid, a sterol and a saturated or unsaturated fatty acid. The lipid may include a plurality of lipids.

The phospholipid refers to a lipid having a phosphate ester in the structure. The phospholipid may be a type of phospholipid that can constitute cell membrane. Examples of the phospholipid include phosphatidylcholine (for example, dioleoylphosphatidylcholine, dilauroyl phosphatidyl choline, dimyristoylphosphatidylcholine, dipalmitoylphosphatidylcholine, distearoylphosphatidylcholine), phosphatidyl glycerol (for example, dioleoyl phosphatidyl glycerol, dilauroyl phosphatidyl glycerol, dimyristoyl phosphatidyl glycerol, dipalmitoyl phosphatidyl glycerol, distearoylphosphatidylglycerol), phosphatidyl ethanolamine (for example, dilauroyl phosphatidyl ethanolamine, dimyristoylphosphatidylethanolamine, dipalmitoyl phosphatidyl ethanolamine, distearoylphosphatidiethanolamine), phosphatidylserine, phosphatidylinositol, phosphatidic acid, cardiolipin, sphingomyelin, ceramide phosphoryl ethanolamine, ceramide phosphoryl glycerol, ceramide phosphoryl glycerol phosphate, 1,2-dimyristoyl-1,2-deoxyphosphatidylcholine, dioleoyl phosphatidyl ethanolamine, soybean phosphatidylcholine, plasmalogen, egg yolk lecithin, soybean lecithin, hydrogenated products of these, 3β-[N—(N′—,N′-dimethylaminoethane)-carbamoyl] cholesterol (DC-Chol), 1,2-dioleoyl-3-trimethylammonium propane (DOTAP) and 1,2-di-O-octadecenyl-3-trimethylammonium propane (DOTMA).

The glycolipid refers to a lipid to which a sugar is bound. In the glycolipid, the sugar can be bound to an end of a lipid. Examples of the glycolipid include glyceroglycolipid (for example, sulfoxyribosyl glyceride, diglycosyl diglyceride, digalactosyl diglyceride, galactosyl diglyceride, glycosyl diglyceride) and glycosphingolipid (for example, galactosyl cerebroside, lactosyl cerebroside, ganglioside). Examples of the sterol include an animal derived sterol (for example, cholesterol, cholesterol succinate, cholestanol, lanosterol, dihydrolanosterol, desmosterol, dihydrocholesterol), a plant derived sterol (phytosterol) (for example, stigmasterol, sitosterol, campesterol, brassicasterol) and a microorganism derived sterol (for example, zymosterol, ergosterol).

The sterol refers to a steroid alcohol present in animal and plant kingdoms. Examples of the sterol include an animal derived sterol (for example, cholesterol, cholesterol succinate, cholestanol, lanosterol, dihydrolanosterol, desmosterol, dihydrocholesterol) and a plant derived sterol (for example, stigmasterol, sitosterol, campesterol, brassicasterol). A microorganism derived sterol such as zymosterol and ergosterol is also included in sterol.

In an embodiment, a mixture of 1,2-dioleoyl-sn-glycero-3-phosphoethanolamine (DOPE), sphingomyelin (SM), and 1,2-dimrystoyl-sn-glycerol, metoxy polyethylene glycol can be used so as to prepare a lipid membrane-based vesicle. In an embodiment, alkylated polyarginine or S2 peptide such as stearylated octaarginine (STR-R8) or S2 peptide can be further included in the vesicle.

The ethanol solution may have an ethanol concentration of, for example, 10 V/V % to 50 V/V % or 10 V/V % to 20 V/V % as long as the solution can solubilize the lipid constituents.

As the mitochondria, isolated mitochondria can be used. The isolation refers to taking out something from a cell. As the mitochondria, mitochondria purified can be used. The purification refers to completely or partially separating a component from at least one of other components after isolation. The mitochondria purified, since they have been already isolated, can be isolated mitochondria. In the specification, the mitochondria isolated referred to as isolated mitochondria and the mitochondria purified is sometimes referred to as purified mitochondria.

Mitochondria can be isolated from cells by shear stress of water, for example, homogenization Mitochondria can be also isolated from cells by breaking cell membrane by repeating a freeze-thaw process. Mitochondria can be also isolated from cells by breaking cell membrane with a surfactant (concentration of critical micelle concentration or more). Mitochondria can be also isolated from cells to outside of cells by bringing them into contact with a surfactant (concentration below critical micelle concentration) and thereafter incubating the treated cells on ice, optionally followed by giving shear stress of water to the treated cells. The shear stress can be provided to the treated cells, preferably without bubbling, for example, by pipetting the solution containing the treated cells. The surfactant having a concentration below critical micelle concentration can be advantageously used for isolation since damage of the mitochondria taken out with the surfactant can be minimized.

The isolated mitochondria can be collected by centrifugation. Mitochondria can be separated from cell components (for example, high-density cell components) such as nuclei, by a centrifuge process performed, for example, at 500×g for few minutes (for example, 4 minutes). Accordingly, mitochondria can be collected by recovering the supernatant after the centrifuge process. Also, mitochondria may be further centrifuged to precipitate. In this manner, mitochondria may be separated from other cell components (for example, small-density cell components) not precipitated.

An aqueous solution containing mitochondria can be maintained in a buffer (for example, mitochondria preservation buffer). The maintenance can be performed at 4° C. Examples of the buffer that can be used include physiological saline, a Tris buffer, a Hepes buffer and a phosphate buffer. The buffer may comprise a tonicity agent. Examples of the tonicity agent may include sugar such as sucrose. The buffer may comprise a divalent ion chelating agent (for example, for example, ethylenediaminetetraacetic acid (EDTA) and glycol ether diaminetetraacetic acid. The buffer may comprise a physiologically acceptable salt (for example, sodium chloride, magnesium chloride).

Lipid membrane-based vesicles encapsulating mitochondria can be obtained by bringing an aqueous solution comprising mitochondria and an organic phase comprising a lipid that can form lipid membrane (for example, ethanol solution) into contact with each other in a confluent channel of a micro flow channel device to mix them. At this time, the present inventors found that when isolated mitochondria having a particle size of about 1 μm are provided to the micro flow channel device, lipid membrane-based vesicles encapsulating mitochondria that have a particle size substantially smaller than 1 μm can be obtained; and that if mitochondria contained in the vesicles are introduced into cells, the mitochondrial activity of the cells can be improved. The obtained lipid membrane-based vesicles encapsulating mitochondria have a smaller PDI than the mitochondria isolated and the monodispersity thereof as an organelle was relatively high. Thus, according to the present invention, there is provided a method for producing a composition (or formulation) comprising a population of lipid membrane-based vesicles encapsulating mitochondria. According to the present invention, there is also provided a composition comprising a population of lipid membrane-based vesicles encapsulating mitochondria or a mitochondrial formulation comprising the population. A composition comprising a group of mitochondria not encapsulated into a vesicle can be obtained by contacting an organic phase containing no lipid with an aqueous solution containing mitochondria at the step of contacting. Such a composition comprising non-encapsulated mitochondria may further be subjected to encapsulating procedures to form a lipid membrane-based vesicle encapsulating mitochondria.

The flow rates of an aqueous solution comprising mitochondria and an organic phase comprising a lipid that can form lipid membrane (for example, ethanol solution) to be introduced into a confluent channel of a micro flow channel device and the flow rate ratio thereof can be appropriately determined by those skilled in the art.

In an embodiment, a population of mitochondria not encapsulated in a vesicle. In an embodiment, the present invention provides a composition or pharmaceutical composition comprising a population of mitochondria not encapsulated in a vesicle. In a preferred embodiment, a population of mitochondria have a particle size distribution, as determined by dynamic light scattering, having a peak at less than 1 μm, 900 nm or less, 800 nm or less, 700 nm or less, 600 nm or less, 500 nm or less, 400 nm or less, or 300 nm or less. The PDI of the population of mitochondria may be 0.5 or less, 0.4 or less, or 0.3 or less. The population may be monodispersed. Such a population can be obtained by dividing isolated mitochondria into smaller ones by iMIT with a micro fluid device. Small mitochondria are thought to be useful in preparing a lipid membrane-based vesicle encapsulating the divided isolated mitochondria and also in administration in a parenteral route in order to avoid any occlusion in a vasculature. Thus, the pharmaceutical composition may be formulated for a parenteral administration (e.g., intravenous, intramuscular, intraventricular, and intracerebroventricular), for example, by encapsulating the divided isolated mitochondria into a lipid membrane-based vesicle. In an embodiment, divided Q may have cristae comparable to undivided Q in number and/or density.

In a preferred embodiment, a mitochondrion encapsulated in a lipid membrane-based vesicle can be a divided one that can be divided upon being encapsulated into a lipid membrane structure (i.e., vesicle or sac). The division of a mitochondrion can be achieved by using a micro fluid device in the presence or absence of a lipid. Thus, the lipid membrane-based vesicle can encapsulate a divided mitochondrion, which may have a peak that is smaller than 1000 nm, 900 nm or less, 800 nm or less, 700 nm or less, 600 nm or less, 500 nm or less, 400 nm or less, or 300 nm or less in a size distribution of the population. In an embodiment, the divided mitochondria have cristae and matrix inside the mitochondria. In a specific embodiment, the divided mitochondria are fulfilled with cristae inside the inner membrane. According to the present invention, such a divided mitochondrion does not necessarily have a membrane potential, but can improve the mitochondrial respiration inside cells by introducing the vesicle into the cells. In an embodiment, the divided isolated mitochondria inside a vesicle may have no detectable membrane potential. The membrane potential of the mitochondria can be detected by using a mitochondrial membrane potential indicator.

In an embodiment, 50% or more, 60% or more, 70% or more, 80% or more, or 90% or more of a population of encapsulated mitochondria consists of divided isolated mitochondria.

In an embodiment, the mitochondrion in the population can be encapsulated into a lipid membrane-based vesicle. In an embodiment, 50% or more, 60% or more, 70% or more, 80% or more or 90% or more of the mitochondria in the population are each encapsulated into a lipid membrane-based vesicle. the encapsulated mitochondria can be formulated as a pharmaceutical composition.

In an embodiment of the present invention, the composition comprising a population of lipid membrane-based vesicles encapsulating mitochondria of the present invention or a mitochondrial formulation comprising the population may have a particle size distribution, as determined by dynamic light scattering, having a peak at less than 1 μm, 900 nm or less, 800 nm or less, 700 nm or less, 600 nm or less, 500 nm or less, 400 nm or less, or 300 nm or less. The composition comprising a population of lipid membrane-based vesicles encapsulating mitochondria of the present invention or a mitochondrial formulation comprising the population may have a particle size distribution, as determined by dynamic light scattering, having a peak at, for example, 200 nm or more and less than 500 nm.

In an embodiment of the present invention, the composition comprising a population of lipid membrane-based vesicles encapsulating mitochondria of the present invention or a mitochondrial formulation comprising the population may have a particle-size distribution, as determined by dynamic light scattering, having a peak at 50 nm to 200 nm, 50 nm to 150 nm, 100 nm to 500 nm, 200 nm to 400 nm, 300 nm to 500 nm, 500 nm to 1500 nm, 600 nm to 1400 nm, 700 nm to 1300 nm, 800 nm to 1200 nm, or about 1000 nm (or about 1 μm).

In another embodiment, a mitochondrion or divided mitochondrion encapsulated into a lipid membrane-based vesicle itself may have no substantial membrane potential or keep no substantial respiration capability in the vesicle. Even if an encapsulated mitochondrion or divided mitochondrion have no substantial membrane potential or no substantial respiration capability in the vesicle, an encapsulated mitochondrion or divided mitochondrion can likely deliver their constituent ingredients, such as mitochondrial DNA, coenzyme Q10, and other ingredients, to a cellular mitochondrion, and thus, can be beneficial to the cell that has accepted the ingredients. A membrane potential or respiration capability of a mitochondrion can decrease under a higher pH conditions such as pH 8 to 10 or 8 to 9. Such an encapsulated mitochondrion or divided mitochondrion can optionally show or recover its physiological function such as membrane potential or respiration after re-introduced into a cell.

A mitochondria DNA that has leaked out from mitochondria will cause some negative impact on cellular function. Further, a mitochondria DNA in isolated mitochondria will improve the mitochondrial function, especially in a cell having a defect in mitochondrial function. Thus, in a preferred embodiment, the divided mitochondria may maintain the mitochondria DNAs inside the mitochondria. Non-nucleic acid components in isolated mitochondria will improve the intracellular mitochondrial function. Thus, in a preferred embodiment, the divided mitochondria may maintain the non-nucleic acid components inside or on the mitochondria.

The particle size distribution of a population of lipid membrane-based vesicles encapsulating mitochondria of the present invention or the upper limit of its peak may vary depending on the cells serving as a source and the isolation method of mitochondria from the cells.

The composition comprising a population of lipid membrane-based vesicles encapsulating mitochondria of the present invention or a mitochondrial formulation comprising the population may have a polydispersity index (PDI), as determined by dynamic light scattering, of 0.5 or less, 0.4 or less, or 0.3 or less. The composition comprising a population of lipid membrane-based vesicles encapsulating mitochondria of the present invention or a mitochondrial formulation comprising the population may have a PDI of 0.2 to 0.4.

The composition comprising a population of lipid membrane-based vesicles encapsulating mitochondria of the present invention or a mitochondrial formulation comprising the population may have a positive, zero or negative zeta potential. In order to improve dispersibility of vesicles in a solution (to prevent aggregation in solution), the zeta potential can be positive or negative. The vesicles may have a plus zeta potential. Due to the plus zeta potential that the vesicles have, uptake by cells can be improved. The composition comprising a population of lipid membrane-based vesicles encapsulating mitochondria of the present invention or a mitochondrial formulation comprising the population may have a zeta potential of, for example, −10 mV or less, −11 mV or less, −12 mV or less,−13 mV or less, −14 mV or less, −15 mV or less, −16 mV or less, −17 mV or less, −18 mV or less, −19 mV or less, or −20 mV or less. The composition comprising a population of lipid membrane-based vesicles encapsulating mitochondria of the present invention or a mitochondrial formulation comprising the population may have a zeta potential of, for example, 10 mV or more, 11 mV or more, 12 mV or more, 13 mV or more, 14 mV or more, 15 mV or more, 16 mV or more, 17 mV or more, 18 mV or more, 19 mV or more, or 20 mV or more. In order for a vesicle population to have a plus zeta potential, the lipid membrane may be formed of a material that can give the plus zeta potential (for example, lipid membrane formed of a cationic lipid and an electrically neutral lipid comprising a lipid having a cationic part (for example, a lipid having cationic part such as stearylated octaarginine and S2 peptide)). In order for a vesicle population to have a minus zeta potential, the lipid membrane may be formed of a material having a minus zeta potential (for example, lipid membrane formed of anionic lipid).

The composition comprising a population of lipid membrane-based vesicles encapsulating mitochondria of the present invention or a mitochondrial formulation comprising the population may have a particle size distribution, as determined by dynamic light scattering, having a peak at less than 1 μm, a PDI of 0.5 or less, and have a plus zeta potential. The composition comprising a population of lipid membrane-based vesicles encapsulating mitochondria of the present invention or a mitochondrial formulation comprising the population may have a particle size distribution, as determined by dynamic light scattering, having a peak at less than 500 nm, a PDI of 0.5 or less, and have a plus zeta potential. The composition comprising a population of lipid membrane-based vesicles encapsulating mitochondria of the present invention or a mitochondrial formulation comprising the population may have a particle size distribution, as determined by dynamic light scattering, having a peak at less than 500 nm, have a PDI of 0.5 or less, and have a zeta potential of 10 mV or more.

The composition comprising a population of lipid membrane-based vesicles encapsulating mitochondria of the present invention or a mitochondrial formulation comprising the population may have a particle size distribution, as determined by dynamic light scattering, having a peak at less than 1 μm, have a PDI of 0.5 or less, and have a minus zeta potential. The composition comprising a population of lipid membrane-based vesicles encapsulating mitochondria of the present invention or a mitochondrial formulation comprising the population may have a particle size distribution, as determined by dynamic light scattering, having a peak at less than 500 nm, a PDI of 0.5 or less, and have a minus zeta potential. The composition comprising a population of lipid membrane-based vesicles encapsulating mitochondria of the present invention or a mitochondrial formulation comprising the population may have a particle size distribution, as determined by dynamic light scattering, having a peak at less than 500 nm, a PDI of 0.5 or less, and have a zeta potential of −10 mV or less.

The composition comprising a population of lipid membrane-based vesicles encapsulating mitochondria of the present invention or a mitochondrial formulation comprising the population may have a particle size distribution, as determined by dynamic light scattering, having a peak at 500 nm to 1500 nm (or about 1 μm), and have a plus zeta potential. The composition comprising a population of lipid membrane-based vesicles encapsulating mitochondria of the present invention or a mitochondrial formulation comprising the population may have a particle size distribution, as determined by dynamic light scattering, having a peak at 500 nm to 1500 nm (or about 1 μm), a PDI of 0.5 or less, and have a plus zeta potential. The composition comprising a population of lipid membrane-based vesicles encapsulating mitochondria of the present invention or a mitochondrial formulation comprising the population may have a particle size distribution, as determined by dynamic light scattering, having a peak at 500 nm to 1500 nm (or about 1 μm), have a PDI of 0.5 or less, and have a zeta potential of 10 mV or more.

The composition comprising a population of lipid membrane-based vesicles encapsulating mitochondria of the present invention or a mitochondrial formulation comprising the population may have a particle size distribution, as determined by dynamic light scattering, having a peak at 500 nm to 1500 nm (or about 1 μm), and have a minus zeta potential. The composition comprising a population of lipid membrane-based vesicles encapsulating mitochondria of the present invention or a mitochondrial formulation comprising the population may have a particle size distribution, as determined by dynamic light scattering, having a peak at 500 nm to 1500 nm (or about 1 μm), have a PDI of 0.5 or less, and have a minus zeta potential. The composition comprising a population of lipid membrane-based vesicles encapsulating mitochondria of the present invention or a mitochondrial formulation comprising the population may have a particle size distribution, as determined by dynamic light scattering, having a peak at 500 nm to 1500 nm (or about 1 μm), have a PDI of 0.5 or less, and have a zeta potential of −10 mV or less.

The composition comprising a population of lipid membrane-based vesicles encapsulating mitochondria of the present invention or a mitochondrial formulation comprising the population, when it is introduced into cells, releases mitochondria into the cytoplasm and improves mitochondrial function (for example, respiratory activity) within the cells. The improvement of mitochondrial function within the cells can be determined, for example, by measuring the oxygen consumption rate of mitochondria.

The composition comprising a population of lipid membrane-based vesicles encapsulating mitochondria of the present invention or a mitochondrial formulation comprising the population, when it is introduced into cells, releases mitochondria into the cytoplasm and allows the mitochondria released to fuse with endogenous mitochondria. More specifically, the composition comprising a population of lipid membrane-based vesicles encapsulating mitochondria of the present invention or a mitochondrial formulation comprising the population encapsulates mitochondria within lipid membrane-based vesicles. When the composition or formulation is incorporated into cells, it is segregated in endosomes. Mitochondria are protected by the lipid membrane even in the endosomal environment and thereafter can be ejected from the endosomes; more specifically, endosomes are fused with lipid membrane of the vesicles to eject mitochondria out of the lipid membrane. In this manner, the mitochondria separated from whole or part of the lipid membrane can be released in the cytoplasm. The mitochondria separated from whole or part of the lipid membrane can be in contact with other mitochondria (for example, endogenous mitochondria) and fuse with them. The fusion of mitochondria in cells can be confirmed by labeling mitochondria in cells and mitochondria encapsulated in vesicles separately with different fluorescent dyes (distinguishable by wavelength) and examining whether they are co-present in the cells.

In the composition comprising a population of lipid membrane-based vesicles encapsulating mitochondria of the present invention or a mitochondrial formulation comprising the population, the interior and exterior environments of the vesicle are physically, biochemically, and/or physiologically separated by the lipid membrane. More specifically, vesicles have a closed space formed of lipid membrane, which serves as a barrier to inhibit free transportation of substances between the interior and exterior regions. Encapsulation of mitochondria in lipid-membrane based vesicles can be confirmed by observing an image of a hollow space within vesicles of lipid-membrane base by a negative staining method and an electron microscope; and labeling mitochondria and lipid membrane separately with different fluorescent dyes (distinguishable by wavelength) and examining whether they are co-present or not when observed by a microscope.

The composition comprising a population of lipid membrane-based vesicles encapsulating mitochondria of the present invention or a mitochondrial formulation comprising the population encapsulates mitochondria. The mitochondria may have respiratory activity within the cells or in the presence of substrates of the respiratory chain complex. The composition comprising a population of lipid membrane based encapsulating mitochondria of the present invention or a mitochondrial formulation comprising the population can be used for improving reduced mitochondrial function in cells. The composition comprising a population of lipid membrane-based vesicles encapsulating mitochondria of the present invention or a mitochondrial formulation comprising the population can be administered, for example, to a tissue having reduced mitochondrial function. The composition comprising a population of lipid membrane-based vesicles encapsulating mitochondria of the present invention or a mitochondrial formulation comprising the population can be administered, for example, to a tissue damaged by myocardial infarction. The composition comprising a population of lipid membrane-based vesicles encapsulating mitochondria of the present invention or a mitochondrial formulation comprising the population, can be administered, for example, to a subject having a mitochondrial dysfunction. Examples of the mitochondrial dysfunction include a neurodegenerative disorder and neuropsychiatric disorder.

The composition comprising a population of lipid membrane-based vesicles encapsulating mitochondria of the present invention or a mitochondrial formulation comprising the population may comprise an effective amount of the vesicles. The effective amount refers to the amount sufficient to improve mitochondrial function of the cells when the vesicles are introduced into the cells.

The composition comprising a population of lipid membrane-based vesicles encapsulating mitochondria of the present invention or a mitochondrial formulation comprising the population can be provided in a frozen state. The composition comprising a population of lipid membrane-based vesicles encapsulating mitochondria of the present invention or a mitochondrial formulation comprising the population in a frozen state may further comprise a cryoprotectant (for example, glycerol).

The composition comprising a population of lipid membrane-based vesicles encapsulating mitochondria of the present invention or a mitochondrial formulation comprising the population can be stored at 4° C. for 1 day or more, 2 days or more, 3 days or more, 4 days or more, 5 days or more, 6 days or more, a week or more. The stored composition can be used so as to improve the intracellular mitochondria in a cell that is contacted with the composition.

According to the present invention, there is provided a method for producing a composition comprising a population of lipid membrane-based vesicles encapsulating mitochondria or a mitochondrial formulation comprising the population, comprising

bringing an aqueous buffer comprising isolated mitochondria and an organic phase comprising a lipid that can form lipid membrane into contact with each other in the confluent channel of a micro flow channel device to mix them.

The organic phase is not particularly limited as long as it can be removed by dialysis to be later carried out; for example, an ethanol solution may be mentioned.

In the method of the present invention, the micro flow channel device may have a channel for facilitating mixing of solutions in contact with each other in a confluent channel and having at least one bend. In the method of the present invention, the micro flow channel device comprises a flow channel for facilitating mixing of solutions in contact with each other in a confluent channel, the flow channel having a baffle construct.

The method of the present invention may further comprise removing ethanol from the obtained mixture (a solution comprising lipid membrane-based vesicles encapsulating mitochondria). Removing ethanol can be carried out by subjecting the obtained mixture to dialysis using a buffer for preserving mitochondria as an external solution.

The method of the present invention may further comprise adding a pharmaceutically acceptable excipient (for example, a buffer, a tonicity agent, a stabilizer, a dispersant, a salt, and a cryoprotectant) to a solution (buffer solution) comprising lipid membrane-based vesicles encapsulating mitochondria.

In the method of the present invention, isolated mitochondria may be stained with a potential-dependent dye (for example, mitochondrial membrane potential indicator). Examples of the mitochondrial membrane potential indicator that can be used in the present invention include tetramethylrhodamine methyl ester (TMRM), tetramethylrhodamine ethyl ester (TMRE), 3,3′-dihexyloxacarbocyanine iodide (DiOC₆), 6-amino-9-(2-methoxycarbonylphenyl)xanthen-3-ylidene) azanium chloride (rhodamine 123), and 5,5′,6,6′-tetrachloro-1,1′,3,3′-tetraethylbenzimidazolylcarbocyanine iodide (JC-1). JC-1 is accumulated in mitochondria in a membrane potential dependent manner, forms an association at a high concentration and changes from green to red. TMRM and TMRE are each accumulated in mitochondria in a membrane potential dependent manner, and show a high fluorescence intensity at a high concentration. In the present invention, for example, TMRE can be used. Accordingly, if a mitochondrial membrane potential indicator is used, the fluorescence intensity thereof reflects the magnitude of the mitochondrial membrane potential. The relationship between fluorescence intensity and membrane potential can be determined based on a calibration curve previously prepared. In this manner, whether mitochondria-encapsulating lipid membrane-based vesicles encapsulate functional mitochondria or not can be easily evaluated.

In an embodiment, the present disclosure provides mitochondria that have been isolated by a new method, referred to herein as the “detergent and homogenization free (DHF)” method, or alternatively, as the “iMIT” method. As described herein, the mitochondria isolated by the iMIT method are not damaged (e.g., retain inner and outer membrane integrity), and maintain functional capacity (e.g., membrane potential). The mitochondria obtained by the iMIT method are referred to herein as “Q” mitochondria. These mitochondria are suitable for use in treatments for various diseases and disorders including those described herein, e.g., by mitochondrial transplantation. Mitochondrial transplantation is a treatment that is expected to have a utility in a variety of diseases and disorders. Exogenous mitochondria (e.g., Q mitochondria) are internalized into cells in which mitochondria are severely dysfunctional and/or cells in which an influx of highly functional mitochondria is a benefit, to restore and/or enhance mitochondrial function.

In embodiments, the present disclosure provides methods for recovering or isolating mitochondria from cells by treating cells in solution with a surfactant at a concentration below the critical micellar concentration (CMC), removing the surfactant from the solution containing the treated cells, and then incubating the surfactant-treated cells to recover the mitochondria into the solution, thereby recovering the mitochondria from the cells. The method is referred to herein as “iMIT”. Accordingly, provided herein is iMIT, a method for obtaining mitochondria from a cell, comprising:

(A) treating cells with a surfactant at a concentration below the critical micelle concentration (CMC) in a first solution,
(B) removing the surfactants from the first solution to form a second solution, and
(C) incubating the surfactant-treated cells in the second solution to recover mitochondria in the second solution. Additional configurations of (A) to (C) above and of the present method are described below.

According to the method of the present disclosure, cells having mitochondria in their cytoplasm are treated with a surfactant at a concentration below the critical micelle concentration in solution. Thus, in embodiments, the cell membranes are weakened in structural strength but are not permeabilized because of the low concentration of the surfactant, while the mitochondrial membranes are exposed to little or no surfactant and remain intact. In embodiments, the cell membranes may be partially permeabilized, but the mitochondrial membranes are exposed to little or no surfactant due to the low concentration of the surfactant and remain intact.

In embodiments, the solution of (A) may comprise a buffer. Exemplary buffers for use in the methods provided herein include, for example, Tris buffer, HEPES buffer, and phosphate buffer. Buffers may be, for example, pH 6.7-7.6 (e.g., pH 6.8-7.4, pH 7.0-7.4, e.g., pH 7.2-7.4, e.g., pH 7.4). In embodiments, the buffers may include tonicity agents and osmotic modifiers. Exemplary tonicity agents and osmotic modifiers include monosaccharides (e.g., glucose, galactose, mannose, fructose, inositol, ribose, xylose, etc.), disaccharides (e.g., lactose, sucrose, cellobiose, trehalose, maltose, etc.), trisaccharides (e.g., raffinose, melesinose, etc.), polysaccharides (e.g., cyclodextrin, etc.), sugar alcohols (e.g., erythritol, xylitol, sorbitol, mannitol, maltitol, etc.), glycerin, diglycerin, polyglycerin, propyleneglycol, polypropyleneglycol, ethyleneglycol, diethyleneglycol, triethyleneglycol, polyethyleneglycol, and the like. Buffers may also contain a chelating agent, particularly a chelating agent for divalent metals, such as a chelating agent for calcium ion. Chelating agents include, for example, glycol ether diaminetetraacetic acid (EGTA) and ethylenediaminetetraacetic acid (EDTA).

In embodiments, a buffer may be a Tris buffer comprising sucrose and a chelator, wherein the pH is 6.7-7.6 (e.g., pH 6.8-7.4, pH 7.0-7.4, e.g., pH 7.2-7.4, e.g., pH 7.4). In embodiments, the Tris buffer may comprise digitonin or saponin, or another surfactant provided herein. In embodiments, the digitonin or saponin or other surfactant may have a concentration of 20% or less, 15% or less, 14% or less, 13% or less, 12% or less, 11% or less, or 10% or less of the critical micelle concentration. In embodiments, digitonin may be used at concentrations of 400 μM or less, 350 μM or less, 200 μM or less, 150 μM or less, 100 μM or less, 90 μM or less, 80 μM or less, 70 μM or less, 60 μM or less, 50 μM or less, 40 μM or less, or 30 μM or less (e.g., at a concentration of 30 μM). In embodiments, saponins may be used at concentrations of 400 μM or less, 400 μM or less, 350 μM or less, 200 μM or less, 150 μM or less, 100 μM or less, 90 μM or less, 80 μM or less, 70 μM or less, 60 μM or less, 50 μM or less, 40 μM or less, or 30 μM or less (e.g., at a concentration of 30 μM).

In embodiments, the surfactant used in the methods provided herein may be an ionic or a nonionic surfactant. Nonionic surfactants used in the present invention may include, for example, ester, ether, and alkyl glycoside forms. Non-ionic surfactants include, for example, alkyl polyethylene glycols, polyoxyethylene alkylphenyl ethers, and alkyl glycosides. Nonionic surfactants may include Triton-X 100, Triton-X 114, Nonidet P-40, n-Dodecyl-D-maltoside, Tween-20, Tween-80, saponin and/or digitonin. In the treating step (A), at least one of the surfactants selected from the group consisting of Triton-X 100, saponin and digitonin is used. In embodiments, the surfactant is saponin or digitonin.

In embodiments, the treatment step (A), comprises treating the cells with a surfactant at a concentration below the critical micelle concentration. The treatment time of the cells in step (A) may be, for example, 1-30 minutes, for example, 1-10 minutes, or for example, 1-5 minutes, for example, 2-4 minutes, for example, 3 minutes. The treatment of the cells in (A) may be carried out on ice, at 4° C. or at room temperature, or at a temperature between.

In embodiments, the concentration of surfactant in the treatment step (A) can be at a concentration below the critical micelle concentration, e.g., 90% or less, 80% or less, 70% or less, 60% or less, 50% or less, 40% or less, 30% or less, 20% or less, 15% or less, 14% or less, 13% or less, 12% or less, 11% or less, 10% or less, for example, 5-15%, for example, 8-12%, for example 10% of the critical micelle concentration.

In embodiments, the treatment step (A) is a pretreatment of the cells. Without wishing to be bound by theory, it is believed that treatment of the cells with a surfactant below the critical micelle concentration can reduce the strength of the cell membrane; and/or partially or completely eliminates the effect of detergents on intracellular mitochondria.

Thus, in view of minimizing the effect of surfactants on mitochondria, the concentration of surfactant in the solution in which the mitochondria come into contact at least in any step (e.g., each of steps (B) to (E)) during and after recovering the mitochondria from the cell can be below the critical micelle concentration, e.g., less than 10%, less than 5%, less than 4%, less than 3%, less than 2%, or less than 1% or less of the critical micelle concentration; or below the detection limit. In view of minimizing the effects of surfactants on mitochondria, it is preferred that no surfactant should be added to the solution in which the mitochondria come into contact, during and after recovering the mitochondria from the cell.

In embodiments, the cells may be in the form of cells present in a tissue, or they may be isolated from a tissue (e.g., single cells) or a population thereof. The cells isolated from the tissue may be cultured cells, or single cells or a population thereof, obtained by treatment of the tissue or cultured cells with enzymes used to make them be single cells, such as collagenase. Tissues may be chopped, if desired, prior to enzymatic treatment, such as collagenase.

In embodiments, the surfactant can be removed from the solution before mitochondria are recovered from the surfactant-treated cells in (A) in order to reduce the concentration of surfactant in contact with the mitochondria or to sufficiently reduce the surfactant in contact with the mitochondria.

In the removing step (B), removal of surfactants can be performed, for example, by replacing the buffer with a solution containing a lower or reduced concentration of surfactant (preferably a surfactant-free solution) (e.g., a buffer) or adding the solution to the buffer. If the surfactant-treated cells are adherent cells, the buffer containing the surfactant can be removed by aspirating the solution, rinsing the cells in a solution containing a lower or reduced concentration of surfactant (preferably a surfactant-free solution) (e.g., a buffer) if needed, and adding a solution containing a lower or reduced concentration of surfactant (preferably a surfactant-free solution) (e.g., a buffer). If the surfactant-treated cells are floating cells, it is possible to remove the surfactant by centrifuging the cells, removing the supernatant, rinsing the cells in a solution containing a lower or reduced concentration of surfactant (preferably a surfactant-free solution) (e.g., a buffer) if needed, and adding a solution containing a lower or reduced concentration of surfactant (preferably a surfactant-free solution) (e.g., a buffer).

Removal means at least decreasing the concentration of surfactant in the solution in which the mitochondria come into contact, including, for example, less than 10%, less than 5%, less than 4%, less than 3%, less than 2%, or less than 1% or less of the concentration of surfactant; or below the detection limit in the solution in which the mitochondria come into contact. To ensure removal of the surfactant from the solution, (B) may include washing the cells with a solution containing a lower or reduced concentration of surfactant (preferably a surfactant-free solution) (e.g., a buffer).

In (B), in order to remove the surfactant from the solution, the solution added to or exchanged with the solution may preferably be a buffer and may be a buffer as described in (A) above (but a solution containing a lower concentration of surfactant, preferably a solution with no surfactant or undetectable levels of surfactant).

Cells treated in (A) have a reduced plasma membrane strength and can allow mitochondria to be released from the cell interior to the extracellular area merely by incubating them in a solution. However, in the steps before (C), the amount of surfactant contacting the mitochondrion is small, and the effect of surfactant on the mitochondrion is limited, thus the decrease in the intensity of the mitochondrial membrane is also limited and/or the mitochondrial membranes remain intact.

In embodiments, the method comprises obtaining mitochondria that are released into the second solution simply by allowing the cell to stand still in the second solution.

Thus, in the present invention, the surfactant-treated cells can be incubated in solution to release mitochondria from the cell interior to the extracellular area. The term “release” in (C) means that mitochondria exit from the interior of the cell to the outside of the region surrounded by the plasma membrane (e.g., on the solution side or extracellular side).

The solution for use in incubating in (C) (the “second solution”) may be a solution containing a lower concentration of surfactant. In preferred embodiments, the second solution is a surfactant-free solution or a solution with a negligible and/or undetectable amount of surfactant. Solutions for use in incubating in (C) may be, for example, buffers as described in (A) above and may be buffers (with lower concentrations of surfactants than one as described in (A) above (preferably surfactant-free solutions). The solution used in (C) may be a solution comprising, for example, a buffer, an osmotic modifier, and a divalent metal chelator, substantially free of surfactants. As used herein, “substantially free” is used in the sense of not excluding the presence of contamination with an amount of “substantially free ingredient” that cannot be removed or cannot be detected.

In (C), the incubation may be, for example, 1-30 minutes, for example, 5-25 minutes, or for example, 5-20 minutes, for example, 5-15 minutes, for example, 10 minutes. The treatment of the cells in (C) may be carried out on ice, or at room temperature, or at a temperature between them.

In (C), a physical stimulus can be added such that the lipid bilayer of the mitochondrion does not cause mechanical disruption, in order to enhance the recovery of the mitochondria from the cell. Thus, in (C), for example, the incubation can be carried out under shaking or non-shaking conditions. In (C), for example, the incubation can be carried out under stirring or non-stirring conditions. In (C), surfactant treatment makes the cells easier to detach from the adhesive surface, so detachment of the cells from the adhesive surface by mild water flow as described above does not appear to negatively affect the polarization ratio. Alternatively, in (C), the incubation can be carried out to the extent that the cells will not become detached.

In (C), mitochondria recovered in solution can be used in various applications as isolated mitochondrial populations. In embodiments, the present disclosure provides populations of mitochondria produced via the method provided herein, which are referred to herein as “Q” mitochondria. In embodiments, the present disclosure provides individual mitochondrion produced via the method provided herein (i.e., individual Q).

In embodiments, the methods provided herein further comprise (D) purifying mitochondria recovered in solution. Mitochondria can be separated from one or more other cellular components by centrifugation. For example, mitochondria can be purified as supernatants by centrifugation of the mitochondrial population recovered in (C) at 1500 g or less, 1000 g or less, or 500 g or less to precipitate contaminants such as the detached cells contained in the mitochondrial population. The mitochondria can preferably be purified, for example, as supernatants by centrifugation at 500 g. Mitochondria may also be collected as a precipitate by subjecting the resultant supernatant to further centrifugation (e.g., 8000 g to 12000 g) for enrichment and the like. The term “purified” used herein means that the mitochondria are separated from at least one of the other components in solution by the manipulation.

The mitochondrial population obtained in (C) and/or (D) above can be used as an isolated mitochondrial population in various applications.

The method of the present invention may further comprise (E) freezing mitochondria. Freezing can be performed by mildly suspending the mitochondria in a buffer for freezing. The buffer for freezing may be a buffer as described in (A), but not including a surfactant, and may further comprise a cryoprotectant. Exemplary cryoprotectants are known in the art and include, for example, glycerol, sucrose, trehalose, dimethyl sulfoxide (DMSO), ethylene glycol, propylene glycol, diethyl glycol, triethylene glycol, glycerol-3-phosphate, proline, sorbitol, formamide, and polymers. Thus, the mitochondria provided herein can be stored by freezing. In the method of the present disclosure, mitochondria may not be frozen if cryopreservation is not necessary, e.g., the mitochondria may be used when freshly isolated. In other embodiments, the mitochondria may be stored at 4° C.±3° C. or on ice. In embodiments, the mitochondria provided herein produced by the method provided herein may be stored in liquid nitrogen, at about −80° C.±3° C. or lower, about −20° C.±3° C. or lower, or about 4° C.±3° C. In embodiments, the mitochondria may be stored for days, weeks, or months, or longer, and retain the capacity to function after thawing.

In embodiments, the methods provided herein further comprise methods for thawing the mitochondria that have been isolated as provided herein and subsequently frozen. Methods for thawing the mitochondria provided herein comprise thawing the mitochondria at a temperature of about 20° C.±3° C. or colder, and thawing the mitochondria rapidly, for example, within about 5, about 4, about 3, about 2, or about 1 minute. In embodiments, the rapid thaw of the mitochondria results in the mitochondria retaining the functional capabilities described herein.

In embodiments, the methods provided herein do not comprise methods of disrupting the cell membrane in the whole process of collecting mitochondria from a cell in such a manner that the mitochondrial membranes are disrupted. For example, in the methods provided herein, the cells are not disrupted by homogenization during the process of collecting mitochondria from cell. That is, in embodiments, the methods provided herein do not comprise homogenization; in embodiments, the methods comprise homogenization but the homogenization is carried only to the extent that it does not cause any bubbles or bubbles to the solution relative to the cell or tissue. In embodiments, the methods also do not comprise freeze-thawing of cells. Although repeated freeze-thawing of cells is suitable for disrupting the plasma membrane and recovering its contents, and can be used to retrieve mitochondria from the cell, freeze-thawing is believed to also disrupt the mitochondrial lipid bilayer because the membrane potential of the obtained mitochondria is not maintained (as opposed to the method of the present disclosure, in which the mitochondrial membrane potential is maintained).

In embodiments, the methods of the present disclosure do not include other methods of disrupting the cell membrane (e.g., sonication, treatment with a strong stream of water to the extent that a solution produces bubbles, or to the extent that the solution foams) during the whole process of collecting mitochondria from cell. In embodiments, the method of the present disclosure is performed without performing any processes that may substantially cause physical, chemical, or physiological damage to the mitochondria, although a freeze-thaw cycle can be applied to the mitochondria for storage. Thus, the method of the present invention is capable of obtaining mitochondria with minimal damage.

The method of the present invention does not require one or more filtration steps in purifying mitochondria recovered from cells.

In embodiments, the methods provided herein gently separate the mitochondria from the microtubule system without damage to the mitochondria, while the mitochondria are still in the cell. During the incubation period, the mitochondria, which have become non-filamentous in shape due to the detachment of the microtubules from the mitochondrial surface, are able to exit the cell through the surfactant-treated cell membrane. Thus, the mitochondria obtained from the cell via the disclosed method are obtained without ripping and tearing of the mitochondrial membrane or otherwise damaging the structure of the mitochondria. Thus, the isolated mitochondria and populations thereof provided herein are capable of maintaining function after isolation and are vastly more suitable for use in treating disease conditions than any previously described isolated mitochondria.

Accordingly, the methods provided herein differ from conventional methods for isolating mitochondria in important ways, and provide isolated or obtained mitochondria that have surprising and advantageous features relative to mitochondria isolated by conventional methods or any other previously disclosed method.

In a preferred embodiment, mitochondria can be isolated from cardiac cells, muscle cells, cardiac muscle cells, cardiac progenitor cells, cardiac stem cells, cell lines such as HUVEC cells, and HeLa cells.

The present disclosure provides a population of isolated or obtained or processed mitochondria, wherein the mitochondria in the population exhibit superior functional capability. For example, in an aspect, the present disclosure provides a population of isolated mitochondria wherein at least about 60%, at least about 65%, at least about 70%, at least about 75%, at least about 80%, at least about 85%, at least about 90%, or at least about 95% of the mitochondria in the population have intact inner and outer membranes; and/or at least about 60%, at least about 65%, at least about 70%, at least about 75%, at least about 80%, at least about 85%, at least about 90%, or at least about 95% of the mitochondria in the population are polarized as measured by a fluorescence indicator. In embodiments, the fluorescence indicator is selected from the group consisting of positively charged dyes such as JC-1, tetramethylrhodamine methyl ester (TMRM), and tetramethylrhodamine ethyl ester (TMRE).

In embodiments, the present disclosure provides a population of isolated mitochondria, wherein at least about 60%, at least about 65%, at least about 70%, at least about 75%, at least about 80%, at least about 85%, at least about 90%, or at least about 95% of the mitochondria in the population maintain functional capability (e.g., are polarized) in an extracellular environment. In embodiments, the functional capability in an extracellular environment is measured by a fluorescence indicator of membrane potential. In embodiments, the fluorescence indicator is selected from the group consisting of positively charged dyes such as JC-1, TMRM, and TMRE. In embodiments, the extracellular environment may comprise a total calcium concentration of about 4 mg/dL to about 12 mg/dL, or about 1 mmol/L (1000 μM) to about 3 mmol/L (3000 μM). For example, in embodiments, the extracellular environment comprises a concentration of total calcium of about 8 mg/dL to about 12 mg/dL, or about 2 mmol/L (2000 μM) to about 3 mmol/L (3000 μM). In embodiments, the extracellular environment comprises a concentration of free or active calcium of about 4 mg/dL to about 6 mg/dL, or about 1 mmol/L (1000 μM) to about 1.5 mmol/L (1500 μM). In embodiments, the population of mitochondria maintain functional capability in an environment having a higher calcium concentration compared to the calcium environment in a cell.

In embodiments, provided herein is a population of isolated mitochondria wherein at least about 60%, at least about 65%, at least about 70%, at least about 75%, at least about 80%, at least about 85%, at least about 90%, or at least about 95% of the mitochondria in the population are not undergoing dynamin-related protein 1 (drp1)-dependent division. In embodiments, provided herein is a population of isolated mitochondria having inner and outer membranes, wherein the inner membranes of the mitochondria comprise densely folded cristae.

In embodiments, provided herein is a population of isolated mitochondria wherein at least about 60%, at least about 65%, at least about 70%, at least about 75%, at least about 80%, at least about 85%, at least about 90%, or at least about 95% of the mitochondria in the population have a substantially non-filamentous, non-branched structure or shape. For example, in embodiments, the mitochondria provided herein appear as round, dot-like, globular, irregularly shaped, and/or slightly elongated, or any mixture thereof, when viewed under a microscope. In embodiments, at least about 60%, at least about 65%, at least about 70%, at least about 75%, at least about 80%, at least about 85%, at least about 90%, or at least about 95% of the mitochondria in the population have a longer diameter to shorter diameter ratio of no more than 4:1, no more than 3.5:1, or no more than 3:1. In embodiments, at least about 60%, at least about 65%, at least about 70%, at least about 75%, at least about 80%, at least about 85%, at least about 90%, or at least about 95% of the isolated mitochondria in the population of mitochondria provided herein have a length shorter than the double or triple of the hydrodynamic diameter of the mitochondrion. In this manner, the isolated mitochondria provided herein have a markedly different shape (non-filamentous) when compared to the shape of most mitochondria (filamentous) that are within cells. Thus, in embodiments, the population of mitochondria provided herein has a shape that is distinct from mitochondria that exist in a cell and have not been isolated, in that at least about 60%, at least about 65%, at least about 70%, at least about 75%, at least about 80%, at least about 85%, at least about 90%, or at least about 95% of the mitochondria in the population are non-filamentous in shape. In embodiments, the population of isolated mitochondria provided herein exhibit decreased association with mitochondria-associated membrane (MAM). In embodiments, the association with MAM is measured by expression of glucose regulated protein 75 (GRP75). In embodiments, the population of isolated mitochondria provided herein exhibit about 60%, at least about 65%, at least about 70%, about 60%, about 50%, about 40%, about 30%, or less association with MAM when compared to mitochondria in a cell, and/or mitochondria that have been obtained by a conventional method of isolation such as one involving homogenization and/or high levels of detergent, as further described herein. In embodiments, the population of isolated mitochondria provided herein exhibit a decrease in association with MAM, wherein the decrease is at least about 30%, at least about 40%, at least about 50%, at least about 60%, at least about 70%, or more relative to the association with MAM of mitochondria in a cell or of mitochondria isolated by a conventional method of isolation.

In embodiments, the population of isolated mitochondria provided herein are between about 500 nm and about 3500 nm in size. In embodiments, at least about 60%, at least about 65%, at least about 70%, at least about 75%, at least about 80%, at least about 85%, at least about 90%, at least about 95%, or at least about 99% of the mitochondria in the population are between about 500 nm and about 3500 nm in size. In embodiments, the average size of the mitochondria in the population is about 500 nm, about 600 nm, about 700 nm, about 800 nm, about 900 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, about 2400 nm, about 2500 nm, about 2600 nm, about 2700 nm, about 2800 nm, about 2900 nm, about 3000 nm, about 3100 nm, about 3200 nm, about 3300 nm, about 3400 nm, or about 3500 nm. In embodiments, the polydispersity index (PDI) of the population of isolated mitochondria is about 0.2 to about 0.8. In embodiments, the PDI of the population of isolated mitochondria is about 0.2 to about 0.5. In embodiments, the PDI of the population of isolated mitochondria is about 0.25 to about 0.35. In embodiments, the zeta potential of the population of mitochondria is about −15 mV to about −40 mV. In embodiments, the zeta potential of the population of mitochondria is about −20 mV, about −25 mV, about −30 mV, about −35 mV, or about −40 mV.

In embodiments, the population of isolated mitochondria provided herein are capable of being incorporated into cells and/or co-localization with endogenous mitochondria in cells, when the population of isolated mitochondria is contacted with a population of cells. For example, in embodiments, the present disclosure provides methods for obtaining mitochondria from cells, and subsequently contacting a population of cells (e.g., ex vivo or in vivo cells) with the population of isolated mitochondria. In such embodiments, the mitochondria provided herein, which are isolated via the iMIT method described herein, are capable of co-localizing with the endogenous mitochondria present in the cells. In embodiments, the mitochondria provided herein are further capable of fusing with the mitochondria present in the cells that they have contacted. In embodiments, a substantial fraction of the population of isolated mitochondria are capable of co-localization and/or fusion with endogenous mitochondria in cells. For example, in embodiments, at least about 30%, at least about 40%, at least about 50%, at least about 60%, at least about 70%, at least about 80%, at least about 90%, or at least about 95% of the mitochondria in the population are capable of co-localization and/or fusion with endogenous mitochondria in cells. Thus, the mitochondria provided herein are markedly different from mitochondria isolated via conventional methods in that they are capable of co-localization and/or fusion with endogenous mitochondria in cells.

In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about 4° C. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about 4° C. In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −20° C. or colder. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −20° C. In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −80° C. or colder. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −80° C. In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage in liquid nitrogen. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage in liquid nitrogen.

In embodiments, the storage is for at least about 2 hours, at least about 6 hours, at least about 12 hours, at least about 24 hours, at least about 48 hours, at least about 1 week, at least about 2 weeks, at least about 3 weeks, at least about 1 month, at least about 2 months, at least about 3 months, or longer. Thus, in embodiments, the isolated mitochondria provided herein are markedly different from mitochondria isolated via conventional methods at least in that they maintain functional capacity when freshly isolated and even after storage.

In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after the population of mitochondria have been frozen for storage and then thawed. In embodiments, after being frozen and then thawed, the maintenance rate of the membrane potential is about 90% relative to the membrane potential of the mitochondria prior to freezing. For example, in embodiments, the polarization ratio of a population of mitochondria that has been frozen and thawed is about 90% of the polarization ratio of that population prior to freezing. In embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after being frozen for storage and then thawed, for example, after being frozen for storage and then thawed one, two, three, or more times. Thus, in embodiments, the isolated mitochondria provided herein are markedly different from mitochondria isolated via conventional methods at least in that they maintain functional capacity when even after being frozen for storage and then thawed.

In embodiments, the population of isolated mitochondria provided herein are capable of being incorporated into cells and/or co-localization with and/or fusion with endogenous mitochondria in cells after storage of the mitochondria at any temperature provided herein (e.g., 4° C.±3° C., −20° C.±3° C., −80° C.±3° C., or in liquid nitrogen). For example, in embodiments, at least about 60%, at least about 65%, at least about 70%, at least about 75%, at least about 80%, at least about 85%, at least about 90%, or at least about 95% of the mitochondria in the population capable of being incorporated into cells and/or co-localization with and/or fusion with endogenous mitochondria in cells after the mitochondria have been stored and/or undergone one or more freeze-thaw cycle. In embodiments, the method of storing and thawing the population of isolated mitochondria provided herein comprises storing the population at about −20° C.±3° C., about −80° C.±3° C., or colder (e.g., in liquid nitrogen), and then thawing the mitochondria at about 20° C.±3° C. or colder, wherein the mitochondria are thawed within about 5 minutes, about 4 minutes, about 3 minutes, about 2 minutes, or about 1 minute. In particular embodiments, the population of mitochondria is thawed within about 1 minute. Thus, in embodiments, the mitochondria provided herein are markedly different from mitochondria isolated via conventional methods at least in that they are capable of being incorporated into cells and/or co-localization with and/or fusion with endogenous mitochondria in cells, whereas mitochondria isolated by conventional methods are incapable of or exhibit vastly reduced ability to being incorporated into cells and/or co-localize with and/or fuse with endogenous mitochondria in cells. In embodiments, the co-localized isolated mitochondria can form a filamentous structure, a network structure, and/or a mesh-like structure.

In embodiments, the present disclosure provides compositions comprising the isolated mitochondria provided herein. The compositions, in embodiments, further comprise one or more pharmaceutically acceptable carrier.

In embodiments, the present disclosure provides methods for isolating mitochondria from cells which differs from conventionally known methods and results in mitochondria having the superior functionality and other characteristics provided herein. In embodiments, the method for isolating mitochondria from cells comprises treating cells in a first solution with a surfactant at a concentration below the critical micelle concentration (CMC) for the surfactant, removing the surfactant to form a second solution, incubating the cells in the second solution, and recovering mitochondria from the second solution. In embodiments the concentration of the surfactant in the first solution is about 50% or less of the CMC for the surfactant. For example, in embodiments, the concentration of the surfactant in the first solution is about 40% or less, about 30% or less, about 20% or less, or about 10% or less of the CMC for the surfactant.

In embodiments, the surfactant is a non-ionic surfactant. In embodiments, the surfactant is selected from the group consisting of Triton-X 100, Triton-X 114, Nonidet P-40, n-Dodecyl-D-maltoside, Tween-20, Tween-80, saponin and digitonin. In embodiments, the surfactant is saponin or digitonin. In embodiments, the concentration of the surfactant is less than about 400 W. For example, in embodiments, the concentration of surfactant in the first solution is less than about 300 μM, less than about 200 μM, less than about 100 μM, or less than about 50 W. In embodiments, the concentration of the surfactant in the first solution is about 100 μM, about 75 μM, about 60 μM, about 50 μM, about 40 μM, about 30 μM, or about 20 W. In embodiments, the concentration of the surfactant in the first solution is about 20 μM to about 50 μM, or about 30 μM to about 40 W.

In embodiments, the first solution further comprises a buffer comprising one or more of a tonicity agent, osmotic modifier, or chelating agent. In embodiments, the first solution comprises a tris buffer, sucrose, and a chelator.

In embodiments, the step of treating the cells in the first solution comprising a low concentration of surfactant (e.g., below the CMC for the surfactant) comprises incubating the cells in the first solution for about 2 minutes to about 30 minutes at room temperature. For example, in embodiments, the step of treating cells in the first solution comprises incubating the cells in the first solution for about 2, about 5, about 10, about 15, about 20, about 25, or about 30 minutes. The incubation may be carried out at a temperature of about 4° C. to about 37° C.

In embodiments, the step of removing the surfactant comprises decreasing the surfactant in the solution to less than 10% of the surfactant concentration in the first solution, or to less than 1% of the surfactant concentration in the first solution. In embodiments, the step of removing the surfactant comprises washing the cells with a buffer.

In embodiments, the step of incubating the second solution comprises incubating the cells in the second solution for about 5 minutes to about 30 minutes. For example, in embodiments, the step of incubating the cells in the second solution comprises incubating the cells in the second solution for about 5, about 10, about 15, about 20, about 25, or about 30 minutes. In embodiments, the step of incubating the cells in the second solution is carried out at a temperature of about 4° C.±3° C. or on ice.

In embodiments, the step of recovering the mitochondria from the second solution comprises collecting the supernatant to recover the isolated mitochondria. In embodiments, the step of recovering the mitochondria from the second solution comprises centrifuging the second solution and collecting the supernatant following centrifugation to recover the isolated mitochondria.

In embodiments, the iMIT may be performed on a cell attaching to a culture surface. In embodiments, the iMIT may be performed on a cell attaching to a culture surface without detaching the cell from the surface. In embodiments, the step of recovering the mitochondria from the second solution comprises collecting the supernatant to recover the isolated mitochondria, which can be optionally followed by washing the remaining cell on the culture surface with the second solution or another second solution to combine it with the supernatant.

In embodiments, the methods provided herein further comprise freezing the isolated mitochondria. In embodiments, the methods comprise freezing the mitochondria in a buffer comprising a cryoprotectant (e.g., glycerol). In embodiments, the methods comprise freezing the mitochondria in the buffer in liquid nitrogen. In embodiments, the methods further comprise thawing the mitochondria after freezing. In embodiments, the methods for thawing the mitochondria comprise rapidly thawing the mitochondria, for example, within about 5 minutes or within about 1 minute. In embodiments, the mitochondria are thawed in a warm bath having a temperature of about 20° C.±3° C. to about 37° C.±3° C. In embodiments, the mitochondria are thawed at a temperature of about 20° C.±3° C. or colder.

In embodiments, the present disclosure provides a population of isolated mitochondria obtained by the method provided herein. In embodiments, the method provided herein is the “iMIT” method and the mitochondria obtained by this method are referred to herein as “Q” mitochondria. In embodiments, the present disclosure provides compositions and/or formulations comprising the population of isolated mitochondria obtained by the methods provided herein.

In embodiments, the present disclosure provides methods for treating or preventing a disease or disorder associated with mitochondrial dysfunction, the method comprising contacting cells of a subject with a population of isolated mitochondria provided herein, e.g., the Q mitochondria. In embodiments, the disease or disorder is an ischemia-related disease or disorder. For example, in embodiments, the ischemia-related disease or disorder is selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, and reperfusion injury of a transplanted organ. In embodiments, the disease or disorder is a genetic disorder. In embodiments, the disease or disorder is a cancer, cardiovascular disease, ocular disorder, otic disorder, autoimmune disease, inflammatory disease, or fibrotic disorder. In embodiments, the disease is acute respiratory distress syndrome (ARDS). In embodiments, the disease or disorder is an aging disease or disorder, or a condition associated with aging. In embodiments, the disease or disorder is pre-eclampsia or intrauterine growth restriction (IUGR).

In embodiments, the present disclosure provides methods for treating or preventing a disease or disorder provided herein, wherein the method comprises administering the population of isolated mitochondria or the composition to a subject in need thereof. In embodiments, the route of administration of the isolated mitochondria is via an intravenous, intra-arterial, intra-tracheal, subcutaneous, intramuscular, inhalation, or intrapulmonary route of administration. In embodiments, the subject is a mammal, e.g., a human.

In embodiments, the present disclosure provides an isolated mitochondrion having intact inner and outer membranes, wherein the inner membrane comprises folded cristae, wherein the mitochondrion has been isolated from a cell, wherein the mitochondrion is polarized as measured by a fluorescence indicator (e.g., JC-1, TMRM, or TMRE), and wherein the mitochondrion is capable of maintaining polarization in an extracellular environment. In embodiments, the folded cristae are densely folded cristae. In embodiments, the mitochondrion has a substantially non-filamentous shape. In embodiments, the mitochondrion comprises voltage dependent anion channels (VDAC) on its surface that are associated with tubulin. For example, in embodiments, the isolated mitochondrion comprises dimeric tubulin associated with VDAC on the surface. In embodiments, the tubulin comprises at least α-tubulin. In embodiments, the tubulin is a heterodimer comprising α-tubulin and β-tubulin. In embodiments, the tubulin is a homodimer. In embodiments, the isolated mitochondrion exhibits decreased association with MAM as measured by GRP75 expression. For example, in embodiments, isolated mitochondrion exhibits about 70%, about 60%, about 50%, about 40%, about 30%, or less association with MAM when compared to mitochondrion that is present in a cell (i.e. has not been isolated), and/or a mitochondrion that has been obtained by a conventional method of isolation such as one involving homogenization and/or high levels of detergent, as further described herein. In embodiments, the isolated mitochondrion provided herein exhibits a decrease in association with MAM, wherein the decrease is at least about 30%, at least about 40%, at least about 50%, at least about 60%, at least about 70%, or more relative to the association with MAM of a mitochondrion that is present in a cell (i.e., has not been isolated) and/a mitochondrion that has been isolated by a conventional method of isolation.

In embodiments, the isolated mitochondrion provided herein has a membrane potential of between about −30 mV and about −220 mV. In embodiments, the isolated mitochondrion is non-filamentous in shape. In embodiments, the isolated mitochondrion is not undergoing drp1-dependent division. In embodiments, the isolated mitochondrion is between about 500 nm and 3500 nm in size. For example, in embodiments, the isolated mitochondrion is about 500, about 600, about 700, about 800 nm, about 900 nm, about 1000 nm, about 1100 nm, about 1200 nm, about 1500 nm, about 2000 nm, about 2500 nm, about 3000 nm, or about 3500 nm in size.

In embodiments, the present disclosure provides an isolated mitochondrion obtained by the methods provided herein. In embodiments, the present disclosure provides compositions and formulations comprising an isolated mitochondrion provided herein.

In a preferred embodiment, mitochondria can be isolated from a MITO-Cell. A MITO-Cell is a cell that has been activated by contacting the cell with a MITO-Porter (see WO2018/092839, which is incorporated herein in its entirety by reference). A MITO-Porter comprises a mitochondria-activating agent inside a liposome, which optionally present a mitochondria-targeting signal molecule such as alkylated physiologically acceptable polycation such as polyarginine or S2 peptide, or lipid conjugated to polycation such as polyarginine or S2 peptide (see WO2017/090763 and WO2018/092839, which are incorporated herein in their entirety by reference). Examples of a mitochondria-activating agent include, but not limited to, antioxidants such as resveratrol (3,5,4′-trihydroxy-trans-stilbene), coenzyme Q10, vitamin C, vitamin E, N-acetylcysteine, 2,2,6,6,-tetramethylpiperidine 1-oxyl (TEMPO), superoxide dismutase (SOD) and glutathione, and in particular, resveratrol is preferable (see WO2018/092839). It has been reported that resveratrol can activate SIRT1 of Sirtuin family, which is a family of enzymes having an NAD⁺ dependent histone deacetylase activity. Resveratrol can corm a Sirtuin-AMPK-PPAR-PGC-1α complex to accelerate the transcription of FOXO1 and results in mitochondrial biogenesis. MITO-Cell, a cell treated with MITO-Porter containing a mitochondria-activating agent, has an activated respiration activity and respiration complex activity due to the mitochondria-activating agent such as an antioxidant. It has also been reported that Sirtuin-AMPK-PPAR-PGC-1α axis signal-inducing mitochondrial biogenesis further activate cristae fusion and fission in order to accept the increase in an oxidative phosphorylation reaction due to influx of e⁻ from NAD⁺. The cristae fusion will cause the densification of cristae, resulting in drastic increase in the fluorescence of a mitochondria indicator within the mitochondria. Therefore, theoretically, the Q isolated from MITO-Cell, which has been activated by MITO-Porter (also referred to as “Super Q”), may have cristae with a density higher than the Q from a untreated cell, an antioxidant included in the MITO-Porter such as resveratrol, more nucleoids, and a more activated SIRT3-AMPK-PPAR-PGC-la complex or an activated SIRT1-AMPK-PPAR-PGC-1α, and more transcripts due to the activation of the complex than Q from a untreated cell. Thus, it is thought that an encapsulated Super Q may show an effect stronger than an encapsulated Q after on intracellular mitochondria activation.

In an embodiment, the present invention provides Super Q or mitochondria isolated from cells that has been treated with MITO-Porter. In an embodiment, Super Q can be isolated by iMIT method. In an embodiment, Super Q and encapsulated Super Q may contain the mitochondria activating agent that has been incorporated by using MITO-Porter such as resveratrol. In an embodiment, Super Q and encapsulated Super Q may contain more mitochondria activating agent than in Q and encapsulated Q isolated from untreated cells.

In an aspect, the present disclosure provides populations of mitochondria that have been isolated from a cell using the methods provided herein and as a result, are highly functional. As described above, the novel method of isolation provided herein is referred to interchangeably as the “DHF” method or the “iMIT” method; the mitochondria obtained via the DHF or iMIT method is referred to herein as “Q” mitochondria. The Q mitochondria have been spared from the disruption and membrane destruction that occurs when mitochondria are isolated via conventional methods, and thus are structurally and functionally superior to mitochondria isolated via conventional methods.

In embodiments, the present disclosure provides a population of isolated or obtained mitochondria, wherein the population contains a high proportion of polarized mitochondria (i.e., the population has a high polarization ratio). Thus, the population of mitochondria provided herein comprises a high proportion of mitochondria having membrane potential. In embodiments, the present disclosure provides a population of mitochondria, wherein a high proportion of the mitochondria in the population have intact inner and outer membranes. In embodiments, the presence of intact inner and outer membranes can be determined by the functional activity of the mitochondria, for example, the membrane potential and polarization.

The population of mitochondria provided herein is thus superior from mitochondrial populations obtained from cells using conventional methods, such as methods that involve homogenization and/or freeze-thaw of cells and/or high concentrations of detergents or surfactants, as described above. For example, the mitochondria isolated from cells via conventional methods are necessarily damaged by the isolation process and lose functional capacity. Accordingly, in the present disclosure provides a population of isolated mitochondria having a higher polarization ratio and/or a higher % polarization and/or higher % mitochondria with an intact inner and outer membrane, than a population of mitochondria obtained by conventional methods.

In embodiments, the polarization ratio of the population of isolated or obtained mitochondria may be, for example, 40% or more, 45% or more, 50% or more, 55% or more, 60% or more, 65% or more, 70% or more, 75% or more, 80% or more, or 85% or more.

In embodiments, at least about 60%, at least about 65%, at least about 70%, at least about 75%, at least about 80%, at least about 85%, at least about 90%, at least about 95%, or more of the population of isolated or obtained mitochondria are polarized as measured by a fluorescence indicator. In embodiments, the fluorescence indicator may be any fluorescence indicator known to the person of ordinary skill in the art to be suitable for measuring mitochondrial membrane potential. In embodiments, the fluorescence indicator is selected from the group consisting of JC-1, TMRM, and TMRE.

In embodiments, at least about 60%, at least about 65%, at least about 70%, at least about 75%, at least about 80%, at least about 85%, at least about 90%, at least about 95%, or more of the population of isolated or obtained mitochondria have intact inner and outer membranes. In embodiments, at least about 60%, at least about 65%, at least about 70%, at least about 75%, at least about 80%, at least about 85%, at least about 90%, at least about 95%, or more of the population of isolated or obtained mitochondria have densely folded cristae in the inner membrane. For example, in embodiments the cristae structure of the Q mitochondria resembles that of the cristae structure of mitochondria that are in a cell, i.e., has not been isolated from a cell. The term “densely folded cristae” as used herein means that the mitochondria comprise cristae present at a high density, that is, highly folded cristae. The density of cristae may be assessed using microscopy (e.g., transmission electron or optical microscopy including confocal microscopy). In embodiments, cristae density in mitochondria may be measured by the number of cristae folds per square micrometer, which can be manually determined by counting the number of folds and/or via an automated software program. In embodiments, “high density of cristae,” “densely folded cristae,” and the like means at least about 3, at least about 4, at least about 5, at least about 6, at least about 7, at least about 8, or more cristae (i.e., cristae folds) per square micrometer. Alternatively or additionally, cristae density in mitochondria may be measured by the cristae surface area per mitochondrial volume. Thus, in embodiments, “high density of cristae,” “densely folded cristae,” and the like means that the cristae surface area per mitochondrial volume (μm² μm⁻³) is at least about 20, at least about 25, at least about 30, at least about 35, at least about 40, or more. Methods for determining cristae density are known in the art (see, for example, Segawa et al., “Quantification of cristae architecture reveals time dependent characteristics of individual mitochondria” Life Science Alliance vol. 3 no. 7, June 2020; and Nielsen et al., The Journal of Physiology 595.9 (2017) pp. 2839-47). In embodiments, at least about 60%, at least about 65%, at least about 70%, at least about 75%, at least about 80%, at least about 85%, at least about 90%, at least about 95%, or more of the mitochondria in the population of mitochondria provided herein have at least about 3, at least about 4, at least about 5, at least about 6, at least about 7, at least about 8, or more cristae per square micrometer; and/or have at least about 20 cristae surface area per mitochondrial volume (μm² μm⁻³), at least about 25 μm² μm⁻³, at least about 30 μm² μm⁻³, at least about 35 μm² μm⁻³, at least about 40 μm² μm⁻³, or more. In embodiments, the isolated mitochondria provided herein have average or representative cristae density that is equivalent to and/or not significantly less than the cristae density of mitochondria in the cell type from which the isolated mitochondria were obtained. In embodiments, at least about 60%, at least about 65%, at least about 70%, at least about 75%, at least about 80%, at least about 85%, at least about 90%, at least about 95%, or more of the mitochondria in the population of mitochondria provided herein exhibit cristae density that is equivalent to and/or not significantly less than the average or representative cristae density of mitochondria in the cell type from which the isolated mitochondria were obtained.

In embodiments, the population of isolated mitochondria provided herein have the surprising feature of maintaining functional capability even when exposed to a high calcium (Ca′) environment. In embodiments, the population of isolated mitochondria provided herein maintain functional capability in an extracellular environment due to the methods of isolation provided herein. In embodiments, at least about 60%, at least about 65%, at least about 70%, at least about 75%, at least about 80%, at least about 85%, at least about 90%, at least about 95%, or more of the population of isolated or obtained mitochondria maintain functional capability in an extracellular environment. In embodiments, the extracellular environment comprises a total calcium concentration of about 6 mg/dL to about 14 mg/dL, or of about 8 mg/dL to about 12 mg/dL. In embodiments, the extracellular environment comprises concentration of free/active calcium of about 3 mg/dL to about 8 mg/dL, or of about 4 mg/dL to about 6 mg/dL. Thus, in embodiments, the Q mitochondria provided herein possess the remarkable characteristics of being isolated from a cellular environment with minimal or negligible damage, and retain capacity to function even when exposed to an extracellular environment, e.g., a calcium rich environment that would otherwise be expected to cause damage to the mitochondria and/or significantly inhibit their functional capacity.

Without wishing to be bound by theory, in some embodiments, the ability of the isolated or obtained mitochondria provided herein to maintain functional capability in an extracellular environment is due, in part or in whole, to the association of tubulin with voltage dependent anion channels (VDAC) on the mitochondrial surface. For example, in embodiments, during the iMIT isolation process provided herein, tubulin may associate with all or a substantial number of VDAC on the mitochondrial surface such that mitochondria are capable of maintaining function even in a calcium rich environment (e.g., an extracellular environment comprising about 3 mg/dL to about 14 mg/dL calcium, or more). In embodiments, the association of tubulin with VDAC on the surface of the isolated mitochondria may be determined by detecting the presence of tubulin at the mitochondrial surface, for example by staining.

Without wishing to be bound by theory, in some embodiments, the isolated Q mitochondria provided herein are able to maintain functional capability in an extracellular environment due, in whole or in part, to a depletion of cholesterol, ergosterol, and/or related molecules in the outer membrane of the Q mitochondria during iMIT isolation. That is, cholesterol (which stabilizes VDAC structure) may be depleted to an extent due to contact of a small amount of surfactant with the mitochondrial membrane during the isolation procedure, resulting in isolated mitochondria having VDAC on the surface that have lost some or all function, such that the mitochondria become resistant to extracellular calcium concentrations (e.g., an extracellular environment comprising about 3 mg/dL to about 14 mg/dL calcium, or more). Thus, in embodiments, the isolated mitochondria provided herein comprise a very low level of sterol concentration in the mitochondrial membrane.

In embodiments, the population of isolated or obtained mitochondria further exhibit reduced association with mitochondria-associated membrane (MAM) relative to mitochondria that are in a cell and/or mitochondria that have been isolated or obtained using a conventional method such as one that involves homogenization of cells and/or freeze thaw of cells. In embodiments, the decreased association with MAM is measured by glucose regulated protein GRP75 expression at the surface of the mitochondria.

In embodiments, the isolated mitochondria are substantially non-filamentous in shape. “Non-filamentous” may be used interchangeably with “non-network-like” and the like, and means that the mitochondria do not exhibit the branched and mesh-like network of mitochondria that exist within a cell. In embodiments, rather than having a filamentous, networked, or branched structure, the mitochondria provided herein appear as round, globular, irregularly shaped, and/or slightly elongated, or any mixture thereof, when viewed under a microscope. At lower magnitude, the isolated mitochondria appear as a dot-like structure. In contrast, at lower magnitude, the highly elongated, network, or branched structure of mitochondria in a cell is visible. In embodiments, at least about 60%, at least about 65%, at least about 70%, at least about 75%, at least about 80%, at least about 85%, at least about 90%, at least about 95%, or more of the population of isolated or obtained mitochondria have a long diameter to short diameter ratio of no more than 4:1, no more than 3.5:1, or no more than 3:1. Without wishing to be bound by theory, the shape of the mitochondria isolated via the methods provided herein results from the gentle removal of connections via motor proteins to microtubules while the mitochondria is still in the cell prior to isolation. That is, once the mitochondria are no longer tethered to the microtubules of the cell, they lose the highly elongated and branched/networked shape that they had within the cell to instead form the non-filamentous shape described herein.

In embodiments, at least about 60%, at least about 65%, at least about 70%, at least about 75%, at least about 80%, at least about 85%, at least about 90%, or at least about 95% of the isolated mitochondria in the population of mitochondria provided herein have a length shorter than the double of the hydrodynamic diameter of the mitochondria. In embodiments, the hydrodynamic diameter is about 1 μm, and at least about 60%, at least about 65%, at least about 70%, at least about 75%, at least about 80%, at least about 85%, at least about 90%, or at least about 95% of the isolated mitochondria in the population of mitochondria provided herein have a length of 2 μm or less, 1.9 μm or less, 1.8 μm or less, 1.7 μm or less, 1.6 μm or less, 1.5 μm or less, 1.4 μm or less, or 1.3 μm or less in length of the major axis. In embodiments, hydrodynamic diameter is measured by Dynamic Light Scattering method (DLS). In embodiments, hydrodynamic diameter is a median diameter D₅₀.

In general in a cell, mitochondria are highly elongated in shape or in the form of filamentous, branched structures as described above. Non-filamentous and non-elongated mitochondria generally only exist in a cell when drp1-dependent division, or drp1-dependent fission, is occurring. For this process of mitochondrial fission, interaction with the endoplasmic reticulum causes initial constriction of the mitochondrion. Drp1 proteins are recruited to mitochondria and assemble on its surface to cause further constriction. DYN2 is recruited to carry out the final stage of membrane scission. The resulting mitochondria may generally be spherical in shape. In a cell, such spherical mitochondria may retain spherical shape for a limited period of time before becoming elongated or forming the more typical branch-like structures. In contrast, mitochondria isolated using the iMIT method are non-filamentous in shape without undergoing Drp1 mediated fission. In addition, mitochondria isolated by conventional methods such as methods involving homogenization of a cell yield mitochondria that are non-filamentous and largely rounded or spherical in shape because they have been damaged and torn from the microtubules in the cell that otherwise cause them to maintain an elongated shape. In contrast to mitochondria isolated in such a manner, the mitochondria isolated by the iMIT method provided herein have not undergone damaging removal from microtubules and are not undergoing drp1 mediated fission. Accordingly, the mitochondria of the present disclosure differ from both natural mitochondria in a cell and mitochondria isolated by more conventional methods. For example, in embodiments, the mitochondria provided herein, obtained via the iMIT method, are substantially non-filamentous in shape while at the same time exhibiting a highly functional status (e.g., polarization), intact inner and outer membrane structure including densely folded cristae, and while not undergoing drp1 fission.

In embodiments, the Q mitochondria provided herein, when contacted with a cell or with a population of cells, exhibit the surprising feature of co-localization with endogenous mitochondria within the cell or cells. The Q mitochondria co-localize with the endogenous mitochondria to a much higher degree compared to mitochondria isolated via conventional methods. In embodiments, the Q mitochondria provided herein, when contacted with a cell or with a population of cells, fuse with endogenous mitochondria within the cell or cells. The fusion of the isolated Q mitochondria is a distinct difference from, and advantage over, mitochondria isolated via conventional methods. In embodiments, the mitochondria retain this ability even after storage. Thus, in embodiments, the Q mitochondria provided herein are superior to conventionally isolated mitochondria at least in that they are more efficient at co-localization with and/or fusion with endogenous mitochondria in cells and thus exhibit a superior clinical effect when used to treat any diseases or disorders such as those described herein. This may suggest that the Q mitochondria provided herein have more robust and nearly intact outer membrane, compared to conventionally isolated mitochondria.

In embodiments, the present disclosure provides a population of mitochondria that is isolated or obtained by the methods provided herein. For example, the present disclosure provides a population of mitochondria that is isolated or obtained by a method comprising steps (A) to (C) of the iMIT method as herein described above. In embodiments, the present disclosure provides a population of mitochondria that is isolated or obtained by a method comprising the steps of (A) to (E) as herein described above.

According to the present disclosure, there is provided a composition comprising a population of isolated mitochondria of the present invention. According to the present disclosure, there is provided a mitochondrial formulation comprising a population of isolated mitochondria of the present invention. Compositions comprising a population of isolated mitochondria of the present invention may further comprise a buffer. Mitochondrial formulations comprising a population of isolated mitochondria of the present invention are pharmaceutically acceptable and may further comprise pharmaceutically acceptable additional components, e.g., excipients. A population of isolated mitochondria of the disclosure, or compositions or mitochondrial formulations containing it, may be obtained during the separation process without using cell sorting by flow cytometer such as fluorescence activated cell sorting (FACS). Thus, a population of isolated mitochondria of the present invention, or a composition or mitochondrial preparation containing it, does not contain fluorescent dyes and fluorescent probes (as well as non-fluorescent mitochondrial stains and probes). In embodiments, the composition is a pharmaceutical composition. All of these features of Q can be performed in Super Q preparation.

In embodiment, the detergent treatment may be performed below 10° C., 9° C., 8° C., 7° C., 6° C., 5° C., or 4° C., preferably at a temperature between about 0° C. and about 4° C. or on ice (as long as the sample does not freeze). In embodiments, all of the isolation procedures may be performed at a temperature between about 0° C. and a room temperature, preferably, below 10° C., 9° C., 8° C., 7° C., 6° C., 5° C., or 4° C., preferably at about 4° C. or on ice.

The present disclosure provides a population of mitochondria that is isolated or obtained by the methods provided herein from cells whose mitochondria have been activated. Activation of mitochondria can be achieved by various methods, for example, by contacting the mitochondria with a mitochondria activating agent. Such an activation of mitochondria can be achieved by various methods including MITO-Porter technology. MITO-Porter technology may use a complex of mitochondria targeting carrier and a mitochondria activating agent. In a complex of mitochondria targeting carrier and a mitochondria activating agent, a mitochondria targeting carrier can be covalently or non-covalently connected to a mitochondria activating agent, optionally through a linker. In the complex, a mitochondria targeting carrier such as MTS peptide, polycation, octaarginine, and S2 peptide can be covalently linked to a lipid or a hydrophobic moiety (e.g., hydrocarbon) to be presented on the surface of a lipid membrane-based vesicle such as a liposome via hydrophobic interaction. In an embodiment, a mitochondria targeting carrier in a form of vesicle may encapsulate or contain a mitochondria activating agent. In embodiments, the activated mitochondria have an improved membrane potential and/or an improved respiration activity, which can be evaluated by oxygen consumption rate (OCR), compared to that of untreated mitochondria or mitochondria before a mitochondria activating treatment.

The present disclosure also provides a population of mitochondria that is isolated or obtained by the methods provided herein from cells that has been treated with a MITO-Porter encapsulating a mitochondria activating agent such as resveratrol. In embodiments, the present disclosure provides a population of mitochondria that is isolated or obtained by the methods provided herein from cells, wherein the population of mitochondria comprises the mitochondria activating agent such as resveratrol. The present invention includes the step of introducing a complex of a mitochondria-targeting carrier and a mitochondria activating agent into cells, such as CPCs or non-CPC cells. In embodiments, the cells may not be cardiac cells.

The present invention includes the step of introducing a complex of a mitochondria-targeting carrier and a mitochondria activating agent into cells, such as CPCs or non-CPC cells. In a preferable embodiment, a complex of a mitochondria-targeting carrier and a mitochondria activating agent will be a lipid-membrane based vesicle encapsulating or containing a mitochondria activating agent (i.e., a mitochondria-targeting liposome).

The mitochondria-targeting carrier is one having a function to selectively reach mitochondria as one of intracellular organelles when the carrier is introduced into a cell. Examples of the mitochondria-targeting carrier may include liposoluble cation substances such as Lipophilic triphenylphosphonium cation (TPP) and Rhodamine 123; polypeptides such as Mitochondrial Targeting Sequence (MTS) peptide (Kong, B W. et al., Biochimica et Biophysica Acta 2003, 1625, pp. 98-108) and S2 peptide (Szeto, H. H. et al., Pharm. Res. 2011, 28, pp. 2669-2679); and mitochondria-targeting liposomes such as DQAsome (Weissig, V. et al., J. Control. Release 2001, 75, pp. 401-408), MITO-Porter (Yamada, Y. et al., Biochim Biophys Acta. 2008, 1778, pp. 423-432), DF-MITO-Porter (Yamada, Y. et al., Mol. Ther. 2011, 19, pp. 1449-1456) and modified DF-MITO-Porter modified with S2 peptide (Kawamura, E. et al., Mitochondrion 2013, 13, pp. 610-614). The documents are hereby incorporated by reference regarding production and use of the carriers in the present invention.

A preferred mitochondria-targeting carrier in the present invention is a mitochondria-targeting liposome, and in particular, MITO-Porter, DF-MITO-Porter or modified DF-MITO-Porter is preferable.

The complex of a mitochondria-targeting carrier and a mitochondria activating agent is a substance having a configuration in which a mitochondria-targeting carrier and a mitochondria activating agent behave in a unified manner regardless of whether chemical bonding, physical encapsulation or the like is used to form the complex. For example, when the liposoluble cation lipid or polypeptide is a mitochondria-targeting carrier, a complex of a mitochondria-targeting carrier and a mitochondria activating agent can be formed by bonding the mitochondria-targeting carrier to the mitochondria activating agent using a chemical method such as covalent bonding or ionic bonding in accordance with, for example, Murphy et al.'s method regarding a liposoluble cation substance (G. F. Kelso et al., J. Biol. Chem., 2001, 276, pp. 4588-4596) or a method regarding Szeto peptide as described in JP2007-503461A.

Further, when the mitochondria-targeting carrier is a liposome, a complex of a mitochondria-targeting carrier and a mitochondria activating agent can be formed by chemically bonding the mitochondria activating agent to a surface of a lipid membrane of the liposome, or physically encapsulating the mitochondria activating agent in the liposome, i.e. an internal space blocked by a lipid membrane.

The complex can be introduced into cells, such as CPCs or non-CPC cells, by a method for introduction of the complex into a cell, which is known for the mitochondria-targeting carrier. The complex may be introduced into a cell by, for example, culturing cells, such as CPCs or non-CPC cells, in an appropriate medium containing the complex, or incubating the complex and cells, such as CPCs or non-CPC cells, in the presence of a known substance capable of accelerating uptake of a substance into a cell, such as lipofectamine or polyethylene glycol.

A preferred example of the step of introducing a complex of a mitochondria-targeting carrier and a mitochondria activating agent into cells, such as CPCs or non-CPC cells, in the first aspect of the present invention is a step of introducing a complex into cells, such as CPCs or non-CPC cells, by incubating cells, such as CPCs or non-CPC cells, and a complex which is a mitochondria-targeting liposome encapsulating a mitochondria activating agent, particularly a complex which is MITO-Porter or DF-MITO-Porter having the surface modified with MTS peptide or S2 peptide and encapsulating a mitochondria activating agent.

The mitochondria activating agent is a substance capable of activating a mitochondrial respiratory chain complex (electron transport system), particularly a substance capable of bringing mitochondria into a polarized state in terms of a membrane potential, and in particular, it is preferable to use a substance capable of bringing mitochondria into a hyperpolarized state. Examples of the mitochondria activating agent may include antioxidants such as resveratrol (3,5,4′-trihydroxy-trans-stilbene), coenzyme Q10 (see WO2020/203961A, which is incorporated herein by reference in its entirety), vitamin C, vitamin E, N-acetylcysteine, TEMPO, SOD and glutathione, and in particular, resveratrol is preferable.

The resveratrol that is preferably used in the present invention may be one extracted from a plant by a known method, or one chemically synthesized by a known method such as, for example, Andrus et al.'s method (Tetrahedron Lett. 2003, 44, pp. 4819-4822).

The cells, such as CPCs or non-CPC cells, produced by the method according to the present invention is one of additional aspects of the present invention, and can considerably improve the viability of mice receiving doxorubicin as shown in Examples below.

It is clinically known that administration of doxorubicin, a type of anthracycline-based pharmaceutical agent, causes severe myocardial injury, and mice receiving doxorubicin are used as cardiac failure model mice. Therefore, the cells, such as CPCs or non-CPC cells, produced by the method according to the present invention can be used for treatment and/or prevention of myocardial injury, particularly severe myocardial injury, recovery, protection or suppression of deterioration of the cardiac function, treatment and/or prevention of cardiac failure, or the like.

Another aspect of the present invention relates to a cell population including myocardial stem cells, wherein an average value of ratios of fluorescence intensity of JC-1 dimer to fluorescence intensity of JC-1 monomer (fluorescence intensity of JC-1 dimer/fluorescence intensity of JC-1 monomer) when the cell population is stained with fluorescent dye JC-1 is 1 to 4.

Mitochondria generate a proton concentration gradient inside and outside the membrane under the action of respiratory chain complexes existing in the mitochondria, and come into a polarized state in which there is a membrane potential. When receiving apoptosis, metabolic stress or the like, the polarized mitochondria are turned into a depolarized state in which the membrane potential is reduced. In this way, the state of polarization of mitochondria is a parameter indicating a metabolism activity of mitochondria, and a cell having a large number of polarized mitochondria is considered to be a cell having activated mitochondria.

It is known that fluorescent dye JC-1 (5,5′,6,6′-tetrachloro-1,1′,3,3′-tetraethylbenzimidazolylcarbocyanine iodide), which is a mitochondrial membrane potential probe, is a monomer emitting green fluorescence in depolarized mitochondria, but forms a dimer emitting red fluorescence in polarized mitochondria. Therefore, the ratio of fluorescence intensity between JC-1 monomer and JC-1 dimer is an index indicating a state of polarization of mitochondria. The ratio of fluorescence intensity can be measured by, for example, detecting a fluorescence ratio in accordance with manufacturer's protocol using JC-1 commercially available from Thermo Fisher Scientific, Cosmo Bio Co., Ltd. or the like.

The cell population according to this aspect is a cell population including cells, such as CPCs or non-CPC cells, having activated mitochondria, and the degree of activation of mitochondria of cells, such as CPCs or non-CPC cells, included in the population can be represented by an average value of ratios of fluorescence intensity of JC-1 dimer to fluorescence intensity of JC-1 monomer (fluorescence intensity of JC-1 dimer/fluorescence intensity of JC-1 monomer) when the cell population is stained with JC-1.

The average value of ratios of fluorescence intensity can be determined by measuring a ratio of fluorescence intensity of JC-1 dimer to fluorescence intensity of JC-1 monomer (fluorescence intensity of JC-1 dimer/fluorescence intensity of JC-1 monomer) for each of any number of cells, such as CPCs or non-CPC cells, preferably more than 10 and less than 100 cells, such as CPCs or non-CPC cells, included in the cell population, and calculating an average value of the measured ratios. The average value of ratios of fluorescence intensity of JC-1 dimer to fluorescence intensity of JC-1 monomer in a cell population including CPC having activated mitochondria is more than 1, preferably 1 to 4.

The cell population according to this aspect is a cell population mainly consisting of cells, such as CPCs or non-CPC cells. The cell population can be produced typically by the foregoing method according to the first aspect of the present invention.

The mitochondria that have been isolated from the cells have been treated with the mitochondria activating agent. Therefore, the mitochondria will be preferably isolated from cells that have been treated with a MITO-Porter (or a lipid membrane based vesicle or liposome) encapsulating or containing a mitochondria activating agent.

In an embodiment, mitochondrial DNA concentration in the isolated mitochondria or the divided mitochondria will be 10⁵ to 10⁷ copies/μg protein. The copy number of mitochondrial DNAs can be calculated by quantitative PCR using primer set comprising a forward primer having a sequence of SEQ ID NO: 1 and a reverse primer having a sequence of SEQ ID NO: 2. In an embodiment, mitochondrial transcription factor A (TFAM) can be contained in the isolated mitochondria or the divided mitochondria at a concentration between about 50 ng/mg total protein and about 300 ng/mg total protein (for example, about 100 ng/mg total protein to 250 ng/mg total protein), which can be calculated by ELISA using anti-TFAM antibody. The calculation can be performed by comparing a TFAM standard value or sample.

In an aspect, the present invention provides a method of measuring mitochondria DNA level in encapsulated mitochondria, comprising providing isolated mitochondria, and measuring mitochondria DNA level in the isolated mitochondria. In an embodiment, the method may further comprises measuring an amount of protein in the isolated mitochondria. In an embodiment, the method may further comprises measuring an amount of protein in the isolated mitochondria and calculating the ratio of mitochondria DNA level to the amount of protein. In an embodiment, mitochondria DNA level may be expressed as a copy number or concentration of mitochondria DNA. In a preferable embodiment, the isolated mitochondria may be encapsulated in a vesicle such as a lipid membrane-based vesicle. In an embodiment, the protein amount can be measured by Bradford method. In an embodiment, the DNA amount can be measured by quantitative PCR method. In a preferable embodiment, the step of measuring mitochondria DNA level may comprise amplifying the mitochondria DNA by using the primer set comprising the first primer having a nucleotide sequence set forth in SEQ ID NO: 1 and the second primer having a nucleotide sequence set forth in SEQ ID NO:2. The method may further comprise comparing the measured mitochondria DNA level to a standard value. The standard value may be a value of the functional isolated mitochondria obtained by iMIT method. The mitochondria DNA level in the isolated mitochondria or the encapsulated mitochondria may be indicative of the damage to the mitochondria during isolation process or storage. In an embodiment, if the level is a predetermined value that is 20% or more, 30% or more, 40% or more, 50% or more, 60% or more, 70% or more, 80% or more, or 90% or more of the standard value, the method may further comprise predicting the sample with a level equal to or more than the predetermined value as being a pharmaceutically effective ingredient for a pharmaceutical composition. In other words, the level may be an indicative of the effectiveness, functionality or usefulness of the sample as a pharmaceutically effective ingredient. In an embodiment, the method may further comprise selecting the sample having a level equal to or more than the predetermined value, and/or discarding the sample having a level below the predetermined value. The method may be a method of test the mitochondrial function of a sample including isolated mitochondria or encapsulated mitochondria. Therefore, the method according to the present invention will be useful in predicting the damage to the mitochondria in a sample such as isolated mitochondria sample, stored mitochondria sample, encapsulated mitochondria sample, pharmaceutically acceptable mitochondria formulation, or pharmaceutically acceptable mitochondria preparation. The step of prediction may comprise comparing the DNA level or the ratio above with a predetermined value. The predetermined value will range between 2×10⁵ copy/μg protein and 5×10⁶ copy/μg protein. The lower limit of the predetermined value will be 2×10⁵ copy/μg protein, 3×10⁵ copy/μg protein 4×10⁵ copy/μg protein, 5×10⁵ copy/μg protein, 6×10⁵ copy/μg protein, 7×10⁵ copy/μg protein, 8×10⁵ copy/μg protein, 9×10⁵ copy/μg protein, 1×10⁶ copy/μg protein, 2×10⁶ copy/μg protein, 3×10⁶ copy/μg protein, 4×10⁶ copy/μg protein, or 5×10⁶ copy/μg protein. The upper limit of the predetermined value may be 5×10⁶ copy/μg protein, 4×10⁶ copy/μg protein, 3×10⁶ copy/μg protein, 2×10⁶ copy/μg protein, or 1×10⁶ copy/μg protein.

The present disclosure also provides a population of isolated or obtained or processed mitochondria that have been artificially activated, wherein the mitochondria in the population exhibit superior functional capability. For example, in an aspect, the present disclosure provides a population of isolated mitochondria wherein at least about 60%, at least about 65%, at least about 70%, at least about 75%, at least about 80%, at least about 85%, at least about 90%, or at least about 95% of the mitochondria in the population have intact inner and outer membranes; and/or at least about 60%, at least about 65%, at least about 70%, at least about 75%, at least about 80%, at least about 85%, at least about 90%, or at least about 95% of the mitochondria in the population are polarized as measured by a fluorescence indicator. In embodiments, the fluorescence indicator is selected from the group consisting of positively charged dyes such as JC-1, tetramethylrhodamine methyl ester (TMRM), and tetramethylrhodamine ethyl ester (TMRE).

In embodiments, the present disclosure provides a population of isolated mitochondria, wherein at least about 60%, at least about 65%, at least about 70%, at least about 75%, at least about 80%, at least about 85%, at least about 90%, or at least about 95% of the mitochondria in the population maintain functional capability (e.g., are polarized) in an extracellular environment. In embodiments, the functional capability in an extracellular environment is measured by a fluorescence indicator of membrane potential. In embodiments, the fluorescence indicator is selected from the group consisting of positively charged dyes such as JC-1, TMRM, and TMRE. In embodiments, the extracellular environment may comprise a total calcium concentration of about 4 mg/dL to about 12 mg/dL, or about 1 mmol/L (1000 μM) to about 3 mmol/L (3000 μM). For example, in embodiments, the extracellular environment comprises a concentration of total calcium of about 8 mg/dL to about 12 mg/dL, or about 2 mmol/L (2000 μM) to about 3 mmol/L (3000 μM). In embodiments, the extracellular environment comprises a concentration of free or active calcium of about 4 mg/dL to about 6 mg/dL, or about 1 mmol/L (1000 μM) to about 1.5 mmol/L (1500 μM). In embodiments, the population of mitochondria maintain functional capability in an environment having a higher calcium concentration compared to the calcium environment in a cell.

In embodiments, provided herein is a population of isolated mitochondria wherein at least about 60%, at least about 65%, at least about 70%, at least about 75%, at least about 80%, at least about 85%, at least about 90%, or at least about 95% of the mitochondria in the population are not undergoing dynamin-related protein 1 (drp1)-dependent division. In embodiments, provided herein is a population of isolated mitochondria having inner and outer membranes, wherein the inner membranes of the mitochondria comprise densely folded cristae.

In embodiments, provided herein is a population of isolated mitochondria wherein at least about 60%, at least about 65%, at least about 70%, at least about 75%, at least about 80%, at least about 85%, at least about 90%, or at least about 95% of the mitochondria in the population have a substantially non-filamentous, non-branched structure or shape. For example, in embodiments, the mitochondria provided herein appear as round, dot-like, globular, irregularly shaped, and/or slightly elongated, or any mixture thereof, when viewed under a microscope. In embodiments, at least about 60%, at least about 65%, at least about 70%, at least about 75%, at least about 80%, at least about 85%, at least about 90%, or at least about 95% of the mitochondria in the population have a longer diameter to shorter diameter ratio of no more than 4:1, no more than 3.5:1, or no more than 3:1. In embodiments, at least about 60%, at least about 65%, at least about 70%, at least about 75%, at least about 80%, at least about 85%, at least about 90%, or at least about 95% of the isolated mitochondria in the population of mitochondria provided herein have a length shorter than the double or triple of the hydrodynamic diameter of the mitochondrion. In this manner, the isolated mitochondria provided herein have a markedly different shape (non-filamentous) when compared to the shape of most mitochondria (filamentous) that are within cells. Thus, in embodiments, the population of mitochondria provided herein has a shape that is distinct from mitochondria that exist in a cell and have not been isolated, in that at least about 60%, at least about 65%, at least about 70%, at least about 75%, at least about 80%, at least about 85%, at least about 90%, or at least about 95% of the mitochondria in the population are non-filamentous in shape. In embodiments, the population of isolated mitochondria provided herein exhibit decreased association with mitochondria-associated membrane (MAM). In embodiments, the association with MAM is measured by expression of glucose regulated protein 75 (GRP75). In embodiments, the population of isolated mitochondria provided herein exhibit about 60%, at least about 65%, at least about 70%, about 60%, about 50%, about 40%, about 30%, or less association with MAM when compared to mitochondria in a cell, and/or mitochondria that have been obtained by a conventional method of isolation such as one involving homogenization and/or high levels of detergent, as further described herein. In embodiments, the population of isolated mitochondria provided herein exhibit a decrease in association with MAM, wherein the decrease is at least about 30%, at least about 40%, at least about 50%, at least about 60%, at least about 70%, or more relative to the association with MAM of mitochondria in a cell or of mitochondria isolated by a conventional method of isolation.

In embodiments, the population of isolated mitochondria provided herein are between about 500 nm and about 3500 nm in size. In embodiments, at least about 60%, at least about 65%, at least about 70%, at least about 75%, at least about 80%, at least about 85%, at least about 90%, at least about 95%, or at least about 99% of the mitochondria in the population are between about 500 nm and about 3500 nm in size. In embodiments, the average size of the mitochondria in the population is about 500 nm, about 600 nm, about 700 nm, about 800 nm, about 900 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, about 2400 nm, about 2500 nm, about 2600 nm, about 2700 nm, about 2800 nm, about 2900 nm, about 3000 nm, about 3100 nm, about 3200 nm, about 3300 nm, about 3400 nm, or about 3500 nm. In embodiments, the polydispersity index (PDI) of the population of isolated mitochondria is about 0.2 to about 0.8. In embodiments, the PDI of the population of isolated mitochondria is about 0.2 to about 0.5. In embodiments, the PDI of the population of isolated mitochondria is about 0.25 to about 0.35. In embodiments, the PDI is about 0 to 0.8, preferably about 0 to 0.5, more preferably about 0 to 0.35. In embodiments, the zeta potential of the population of mitochondria is about −15 mV to about −40 mV. In embodiments, the zeta potential of the population of mitochondria is about −20 mV, about −25 mV, about −30 mV, about −35 mV, or about −40 mV.

In embodiments, the population of isolated mitochondria provided herein are capable of being incorporated into cells and/or co-localization with endogenous mitochondria in cells, when the population of isolated mitochondria is contacted with a population of cells. For example, in embodiments, the present disclosure provides methods for obtaining mitochondria from cells, and subsequently contacting a population of cells (e.g., ex vivo or in vivo cells) with the population of isolated mitochondria. In such embodiments, the mitochondria provided herein, which are isolated via the iMIT method described herein, are capable of co-localizing with the endogenous mitochondria present in the cells. In embodiments, the mitochondria provided herein are further capable of fusing with the mitochondria present in the cells that they have contacted. In embodiments, a substantial fraction of the population of isolated mitochondria are capable of co-localization and/or fusion with endogenous mitochondria in cells. For example, in embodiments, at least about 30%, at least about 40%, at least about 50%, at least about 60%, at least about 70%, at least about 80%, at least about 90%, or at least about 95% of the mitochondria in the population are capable of co-localization and/or fusion with endogenous mitochondria in cells. Thus, the mitochondria provided herein are markedly different from mitochondria isolated via conventional methods in that they are capable of co-localization and/or fusion with endogenous mitochondria in cells.

In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about 4° C. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about 4° C. In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −20° C. or colder. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −20° C. In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −80° C. or colder. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage at about −80° C. In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage in liquid nitrogen. For example, in embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after storage in liquid nitrogen. In embodiments, the storage is for at least about 2 hours, at least about 6 hours, at least about 12 hours, at least about 24 hours, at least about 48 hours, at least about 1 week, at least about 2 weeks, at least about 3 weeks, at least about 1 month, at least about 2 months, at least about 3 months, or longer. Thus, in embodiments, the isolated mitochondria provided herein are markedly different from mitochondria isolated via conventional methods at least in that they maintain functional capacity when freshly isolated and even after storage.

In embodiments, the isolated mitochondria provided herein are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after the population of mitochondria have been frozen for storage and then thawed. In embodiments, after being frozen and then thawed, the maintenance rate of the membrane potential is about 90% relative to the membrane potential of the mitochondria prior to freezing. For example, in embodiments, the polarization ratio of a population of mitochondria that has been frozen and thawed is about 90% of the polarization ratio of that population prior to freezing. In embodiments, at least about 60%, at least about 65%, at least about 70%, at least about 75%, at least about 80%, at least about 85%, at least about 90%, or at least about 95% of the mitochondria in the population are stable and/or polarized and/or maintain membrane potential and/or maintain an intact inner and outer membrane and/or maintain the capacity to function after exposure to an extracellular environment (e.g., after exposure to a total calcium concentration of about 4 mg/dL to about 12 mg/dL), after being frozen for storage and then thawed, for example, after being frozen for storage and then thawed one, two, three, or more times. Thus, in embodiments, the isolated mitochondria provided herein are markedly different from mitochondria isolated via conventional methods at least in that they maintain functional capacity when even after being frozen for storage and then thawed.

In embodiments, the population of isolated mitochondria provided herein are capable of being incorporated into cells and/or co-localization with and/or fusion with endogenous mitochondria in cells after storage of the mitochondria at any temperature provided herein (e.g., 4° C.±3° C., −20° C.±3° C., −80° C.±3° C., or in liquid nitrogen). For example, in embodiments, at least about 60%, at least about 65%, at least about 70%, at least about 75%, at least about 80%, at least about 85%, at least about 90%, or at least about 95% of the mitochondria in the population capable of being incorporated into cells and/or co-localization with and/or fusion with endogenous mitochondria in cells after the mitochondria have been stored and/or undergone one or more freeze-thaw cycle. In embodiments, the method of storing and thawing the population of isolated mitochondria provided herein comprises storing the population at about −20° C.±3° C., about −80° C.±3° C., or colder (e.g., in liquid nitrogen), and then thawing the mitochondria at about 20° C.±3° C. or colder, wherein the mitochondria are thawed within about 5 minutes, about 4 minutes, about 3 minutes, about 2 minutes, or about 1 minute. In particular embodiments, the population of mitochondria is thawed within about 1 minute. Thus, in embodiments, the mitochondria provided herein are markedly different from mitochondria isolated via conventional methods at least in that they are capable of being incorporated into cells and/or co-localization with and/or fusion with endogenous mitochondria in cells, whereas mitochondria isolated by conventional methods are incapable of or exhibit vastly reduced ability to being incorporated into cells and/or co-localize with and/or fuse with endogenous mitochondria in cells. In embodiments, the co-localized isolated mitochondria can form a filamentous structure, a network structure, and/or a mesh-like structure.

In embodiments, the present disclosure provides compositions comprising the isolated mitochondria provided herein. The compositions, in embodiments, further comprise one or more pharmaceutically acceptable carrier.

In embodiments, the present disclosure provides methods for isolating mitochondria from cells which differs from conventionally known methods and results in mitochondria having the superior functionality and other characteristics provided herein. In embodiments, the method for isolating mitochondria from cells comprises treating cells in a first solution with a surfactant at a concentration below the critical micelle concentration (CMC) for the surfactant, removing the surfactant to form a second solution, incubating the cells in the second solution, and recovering mitochondria from the second solution. In embodiments the concentration of the surfactant in the first solution is about 50% or less of the CMC for the surfactant. For example, in embodiments, the concentration of the surfactant in the first solution is about 40% or less, about 30% or less, about 20% or less, or about 10% or less of the CMC for the surfactant.

In embodiments, the surfactant is a non-ionic surfactant. In embodiments, the surfactant is selected from the group consisting of Triton-X 100, Triton-X 114, Nonidet P-40, n-Dodecyl-D-maltoside, Tween-20, Tween-80, saponin and digitonin.

In embodiments, the surfactant is saponin or digitonin. In embodiments, the concentration of the surfactant is less than about 400 μM. For example, in embodiments, the concentration of surfactant in the first solution is less than about 300 μM, less than about 200 μM, less than about 100 μM, or less than about 50 μM. In embodiments, the concentration of the surfactant in the first solution is about 100 μM, about 75 μM, about 60 μM, about 50 μM, about 40 μM, about 30 μM, or about 20 μM. In embodiments, the concentration of the surfactant in the first solution is about 20 μM to about 50 μM, or about 30 μM to about 40 W.

In embodiments, the first solution further comprises a buffer comprising one or more of a tonicity agent, osmotic modifier, or chelating agent. In embodiments, the first solution comprises a tris buffer, sucrose, and a chelator.

In embodiments, the step of treating the cells in the first solution comprising a low concentration of surfactant (e.g., below the CMC for the surfactant) comprises incubating the cells in the first solution for about 2 minutes to about 30 minutes at room temperature. For example, in embodiments, the step of treating cells in the first solution comprises incubating the cells in the first solution for about 2, about 5, about 10, about 15, about 20, about 25, or about 30 minutes. The incubation may be carried out at a temperature of about 4° C. to about 37° C.

In embodiments, the step of removing the surfactant comprises decreasing the surfactant in the solution to less than 10% of the surfactant concentration in the first solution, or to less than 1% of the surfactant concentration in the first solution. In embodiments, the step of removing the surfactant comprises washing the cells with a buffer.

In embodiments, the step of incubating the second solution comprises incubating the cells in the second solution for about 5 minutes to about 30 minutes. For example, in embodiments, the step of incubating the cells in the second solution comprises incubating the cells in the second solution for about 5, about 10, about 15, about 20, about 25, or about 30 minutes. In embodiments, the step of incubating the cells in the second solution is carried out at a temperature of about 4° C.±3° C. or on ice.

In embodiments, the step of recovering the mitochondria from the second solution comprises collecting the supernatant to recover the isolated mitochondria. In embodiments, the step of recovering the mitochondria from the second solution comprises centrifuging the second solution and collecting the supernatant following centrifugation to recover the isolated mitochondria.

In embodiments, the iMIT may be performed on a cell attaching to a culture surface. In embodiments, the iMIT may be performed on a cell attaching to a culture surface without detaching the cell from the surface. In embodiments, the step of recovering the mitochondria from the second solution comprises collecting the supernatant to recover the isolated mitochondria, which can be optionally followed by washing the remaining cell on the culture surface with the second solution or another second solution to combine it with the supernatant.

In embodiments, the methods provided herein further comprise freezing the isolated mitochondria. In embodiments, the methods comprise freezing the mitochondria in a buffer comprising a cryoprotectant (e.g., glycerol). In embodiments, the methods comprise freezing the mitochondria in the buffer in liquid nitrogen. In embodiments, the methods further comprise thawing the mitochondria after freezing. In embodiments, the methods for thawing the mitochondria comprise rapidly thawing the mitochondria, for example, within about 5 minutes or within about 1 minute. In embodiments, the mitochondria are thawed in a warm bath having a temperature of about 20° C.±3° C. to about 37° C.±3° C. In embodiments, the mitochondria are thawed at a temperature of about 20° C.±3° C. or colder.

In embodiments, the present disclosure provides a population of isolated mitochondria obtained by the method provided herein. In embodiments, the method provided herein is the “iMIT” method and the mitochondria obtained by this method are referred to herein as “Q” mitochondria. In embodiments, the present disclosure provides compositions and/or formulations comprising the population of isolated mitochondria obtained by the methods provided herein.

In embodiments, the present disclosure provides methods for treating or preventing a disease or disorder associated with mitochondrial dysfunction, the method comprising contacting cells of a subject with a population of isolated mitochondria provided herein, e.g., the Q mitochondria. In embodiments, the disease or disorder is an ischemia-related disease or disorder. For example, in embodiments, the ischemia-related disease or disorder is selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, and reperfusion injury of a transplanted organ. In embodiments, the disease or disorder is a genetic disorder. In embodiments, the disease or disorder is a cancer, cardiovascular disease, ocular disorder, otic disorder, autoimmune disease, inflammatory disease, or fibrotic disorder. In embodiments, the disease is acute respiratory distress syndrome (ARDS). In embodiments, the disease or disorder is an aging disease or disorder, or a condition associated with aging. In embodiments, the disease or disorder is pre-eclampsia or intrauterine growth restriction (IUGR).

In embodiments, the present disclosure provides methods for treating or preventing a disease or disorder provided herein, wherein the method comprises administering the population of isolated mitochondria or the composition to a subject in need thereof. In embodiments, the route of administration of the isolated mitochondria is via an intravenous, intra-arterial, intra-tracheal, subcutaneous, intramuscular, inhalation, or intrapulmonary route of administration. In embodiments, the subject is a mammal, e.g., a human.

In embodiments, the present disclosure provides an isolated mitochondrion having intact inner and outer membranes, wherein the inner membrane comprises folded cristae, wherein the mitochondrion has been isolated from a cell, wherein the mitochondrion is polarized as measured by a fluorescence indicator (e.g., JC-1, TMRM, or TMRE), and wherein the mitochondrion is capable of maintaining polarization in an extracellular environment. In embodiments, the folded cristae are densely folded cristae. In embodiments, the mitochondrion has a substantially non-filamentous shape. In embodiments, the mitochondrion comprises voltage dependent anion channels (VDAC) on its surface that are associated with tubulin. For example, in embodiments, the isolated mitochondrion comprises dimeric tubulin associated with VDAC on the surface. In embodiments, the tubulin comprises at least α-tubulin.

In embodiments, the tubulin is a heterodimer comprising α-tubulin and β-tubulin. In embodiments, the tubulin is a homodimer. In embodiments, the isolated mitochondrion exhibits decreased association with MAM as measured by GRP75 expression. For example, in embodiments, isolated mitochondrion exhibits about 70%, about 60%, about 50%, about 40%, about 30%, or less association with MAM when compared to mitochondrion that is present in a cell (i.e. has not been isolated), and/or a mitochondrion that has been obtained by a conventional method of isolation such as one involving homogenization and/or high levels of detergent, as further described herein. In embodiments, the isolated mitochondrion provided herein exhibits a decrease in association with MAM, wherein the decrease is at least about 30%, at least about 40%, at least about 50%, at least about 60%, at least about 70%, or more relative to the association with MAM of a mitochondrion that is present in a cell (i.e., has not been isolated) and/a mitochondrion that has been isolated by a conventional method of isolation.

In embodiments, the isolated mitochondrion provided herein has a membrane potential of between about −30 mV and about −220 mV. In embodiments, the isolated mitochondrion is non-filamentous in shape. In embodiments, the isolated mitochondrion is not undergoing drp1-dependent division. In embodiments, the isolated mitochondrion is between about 500 nm and 3500 nm in size. For example, in embodiments, the isolated mitochondrion is about 500, about 600, about 700, about 800 nm, about 900 nm, about 1000 nm, about 1100 nm, about 1200 nm, about 1500 nm, about 2000 nm, about 2500 nm, about 3000 nm, or about 3500 nm in size.

In embodiments, the present disclosure provides an isolated mitochondrion obtained by the methods provided herein. In embodiments, the present disclosure provides compositions and formulations comprising an isolated mitochondrion provided herein.

The present disclosure may also provide the invention as described below.

Item 1. A population of isolated mitochondria, wherein:
(i) at least 80% of the mitochondria in the population have intact inner and outer membranes,
(ii) at least 80% of the mitochondria in the population are polarized as measured by a fluorescence indicator,
and/or
(iii) at least 80% of the mitochondria in the population maintain functional capability in an extracellular environment. In a preferable embodiment, the mitochondria have been activated. In a more preferable embodiment, the mitochondria have been activated with a mitochondria activating agent such as resveratrol. In a still more preferable embodiment, such mitochondria can be obtained from cells that has been treated with a mitochondria activating agent, for example, a lipid membrane based vesicle (e.g., liposome) containing or encapsulating a mitochondria activating agent such as resveratrol.
Item 2. The population of isolated mitochondria of Item 1, wherein the functional capability in an extracellular environment of (iii) is measured by a fluorescence indicator of membrane potential.
Item 3. The population of isolated mitochondria of Item 1, wherein the extracellular environment of (iii) comprises a total calcium concentration of about 8 to about 12 mg/dL.
Item 4. The population of isolated mitochondria of Item 1, wherein the extracellular environment of (iii) comprises a free/active calcium concentration of about 4 to about 6 mg/dL.
Item 5. The population of isolated mitochondria of Item 1, wherein at least 80% of the mitochondria in the population are not undergoing dynamin-related protein 1 (drp1)-dependent division.
Item 6. The population if isolated mitochondria of Item 1, wherein the inner membranes of the mitochondria comprise densely folded cristae.
Item 7. The population of isolated mitochondria of any one of Items 1-6, wherein at least 80% of the mitochondria in the population have a non-filamentous shape.
Item 8. The population of isolated mitochondria of Item 7, wherein at least 85% of the mitochondria have a non-filamentous shape.
Item 9. The population of isolated mitochondria of Item 8, wherein at least 90% of the mitochondria have a non-filamentous shape.
Item 10. The population of isolated mitochondria of any one of Items 1-9, wherein the mitochondria exhibit decreased association with mitochondria-associated membrane (MAM) as measured by glucose regulated protein 75 (GRP75) expression.
Item 11. The population of isolated mitochondria of Item 10, wherein the decreased association is a decrease of at least about 30% relative to the association with MAM of mitochondria in a cell or of isolated mitochondria obtained by a method comprising homogenization of cells.
Item 12. The population of isolated mitochondria of Item 11, wherein the decreased association is a decrease of at least about 50%.
Item 13. The population of isolated mitochondria of any one of Items 1-12, wherein
(i) at least 85% of the mitochondria in the population have intact inner and outer membranes,
(ii) at least 85% of the mitochondria in the population are polarized as measured by a fluorescence indicator,
and/or
(iii) at least 85% of the mitochondria in the population maintain functional capability in an extracellular environment.
Item 14. The population of isolated mitochondria of any one of Items 1-12, wherein
(i) at least 90% of the mitochondria in the population have intact inner and outer membranes,
(ii) at least 90% of the mitochondria in the population are polarized as measured by a fluorescence indicator, and/or
(iii) at least 90% of the mitochondria in the population maintain functional capability in an extracellular environment.
Item 15. The population of isolated mitochondria of any one of Items 1-14, wherein the fluorescence indicator is selected from the group consisting of JC-1, tetramethylrhodamine methyl ester (TMRM), and tetramethylrhodamine ethyl ester (TMRE).
Item 16. The population of isolated mitochondria of any one of Items 1-15, wherein at least 80% of the mitochondria in the population are between about 500 nm and about 3500 nm in size.
Item 17. The population of isolated mitochondria of any one of Items 1-16, wherein the polydispersity index (PDI) of the population is about 0.2 to about 0.8, Item 18. The population of isolated mitochondria of any one of Items 1-16, wherein the polydispersity index (PDI) of the population is about 0.2 to about 0.3.
Item 19. The population of isolated mitochondria of any one of Items 1-18, wherein the zeta potential of the population of mitochondria is between about −15 mV and about −40 mV.
Item 20. The population of isolated mitochondria of any one of Items 1-19, wherein upon contact of the population of isolated mitochondria with a population of cells, the isolated mitochondria are capable co-localization with endogenous mitochondria in the cells.
Item 21. The population of isolated mitochondria of any one of Items 1-19, wherein upon contact of the population of isolated mitochondria with a population of cells, the mitochondria are capable of fusing with endogenous mitochondria in the cells.
Item 22. The population of isolated mitochondria of Item 20, wherein the mitochondria are capable of co-localization with the endogenous mitochondria after storage at 4° C. for at least 12 hours.
Item 23. The population of isolated mitochondria of Item 21, wherein the mitochondria are capable of fusing with the endogenous mitochondria after storage at 4° C. for at least 12 hours.
Item 24. The population of isolated mitochondria of any one of Items 1-23, wherein at least 70% of the isolated mitochondria in the population are polarized as measured by a fluorescence indicator, after the population undergoes one or more freeze-thaw cycle.
Item 25. The population of isolated mitochondria of any one of Items 1-24, wherein the mitochondria are capable of co-localization with endogenous mitochondria, after the population undergoes one or more freeze-thaw cycle.
Item 26. The population of isolated mitochondria of Item 24 or 25, wherein the population is frozen at−80° C. or colder for at least two weeks and then thawed at 20° C. or colder within about 5 minutes.
Item 27. The population of isolated mitochondria of Item 26, wherein the population is thawed within about 1 minute.
Item 28. The population of isolated mitochondria of Item 26 or 27, wherein the population is frozen in liquid nitrogen for the at least two weeks.
Item 29. The population of isolated mitochondria of Item 28, wherein the population is frozen in liquid nitrogen for at least two months.
Item 30. The population of isolated mitochondria of any one of Items 24 to 29, wherein upon contact of the thawed population of mitochondria with a population of cells, the isolated mitochondria in the population are capable of fusion with endogenous mitochondria in the cells.
Item 31. A composition comprising the population of isolated mitochondria of any one of Items 1-30.
Item 32. A formulation comprising the composition of Item 31 and a pharmaceutically acceptable carrier.
Item 33. A method for isolating mitochondria from cells, the method comprising:
(i) treating cells in a first solution with a surfactant at a concentration below the critical micellar concentration for the surfactant,
(ii) removing the surfactant to form a second solution,
(iii) incubating the cells in the second solution, and
(iv) recovering mitochondria from the second solution, wherein the cells have activated mitochondria, for example, by contacting the cells with a lipid-membrane based vesicle containing or encapsulating a mitochondria activating agent.
Item 34. The method of Item 33, wherein the concentration of the surfactant in the first solution is about 50% or less of the critical micelle concentration for the surfactant.
Item 35. The method of Item 33 or 34, wherein the concentration of the surfactant in the first solution is about 10% or less of the critical micelle concentration for the surfactant.
Item 36. The method of any one of Items 33-35, wherein the surfactant is a nonionic surfactant.
Item 37. The method of any one of Items 33-36, wherein the surfactant is selected from the group consisting of Triton-X 100, Triton-X 114, Nonidet P-40, n-Dodecyl-D-maltoside, Tween-20, Tween-80, saponin and digitonin.
Item 38. The method of Item 37, wherein the surfactant is saponin or digitonin, and wherein the concentration of the surfactant in the first solution is less than about 400 W.
Item 39. The method of Item 37, wherein the surfactant is saponin or digitonin, and wherein the concentration of the surfactant in the first solution is less than about 50 W.
Item 40. The method of Item 37, wherein the surfactant is saponin or digitonin, and wherein the concentration of saponin or digitonin in the first solution is about 30 μM to about 40 W.
Item 41. The method of any one of Items 33-40, wherein the first solution further comprises a buffer comprising one or more of a tonicity agent, osmotic modifier, or chelating agent.
Item 42. The method of Item 41, wherein the first solution comprises a tris buffer, sucrose, and a chelator.
Item 43. The method of any one of Items 33-42, wherein treating the cells in the first solution comprise incubating the cells in the first solution for about 2 minutes to about 30 minutes at room temperature.
Item 44. The method of any one of Items 33-43, wherein removing the surfactant comprises decreasing the surfactant in the solution to less than 10% of the surfactant concentration in the first solution.
Item 45. The method of any one of Items 33-44, wherein removing the surfactant comprises decreasing the surfactant in the solution to less than 1% of the surfactant concentration in the first solution.
Item 46. The method of any one of Items 33-45, wherein removing the surfactant comprises washing the cells with a buffer.
Item 47. The method of any one of Items 33-46, wherein incubating the second solution comprises incubating the cells in the second solution for about 5 minutes to about 30 minutes at about 4° C.
Item 48. The method of any one of Items 33-47, wherein recovering the mitochondria from the second solution comprises collecting the supernatant to recover the isolated mitochondria.
Item 49. The method of any one of Items 33-48, wherein recovering the mitochondria from the second solution comprises centrifuging the second solution and collecting the supernatant following centrifugation to recover the isolated mitochondria.
Item 50. The method of any one of Items 33-49, wherein the method further comprises freezing the isolated mitochondria.
Item 51. The method of Item 50, wherein the method comprises freezing the isolated mitochondria in a buffer comprising a cryoprotectant.
Item 52. A population of isolated mitochondria obtained by the method according to any one of Items 33-51.
Item 53. A method for treating a disease or disorder, the method comprising contacting cells of a subject in need thereof with a population of isolated mitochondria according to any one of Items 1-30 or a composition of Item 31 or a formulation of Item 32, wherein the disease or disorder is selected from the group consisting of diabetes (Type I and Type II), metabolic disease, ocular disorders associated with mitochondrial dysfunction, hearing loss, mitochondrial toxicity associated with therapeutic agents, cardiotoxicity associated with chemotherapy or other therapeutic agents, a mitochondrial dysfunction disorder, and migraine.
Item 54. A method for treating a disease or disorder associated with mitochondrial dysfunction, the method comprising contacting cells of a subject in need thereof with a population of isolated mitochondria according to any one of Items 1-30 or a composition of Item 31 or a formulation of Item 32.
Item 55. The method of Item 54, wherein the disease or disorder is selected from the group consisting of mitochondrial myopathy, diabetes and deafness (DAD) syndrome, Barth Syndrome, Leber's hereditary optic neuropathy (LHON), Leigh syndrome, NARP (neuropathy, ataxia, retinitis pigmentosa and ptosis syndrome), myoneurogenic gastrointestinal encephalopathy (MNGIE), MELAS (mitochondrial encephalopathy, lactic acidosis, and stroke-like episodes) syndrome, myoclonic epilepsy with ragged red fibers (MERRF) syndrome, Kearns-Sayre syndrome, and mitochondrial DNA depletion syndrome.
Item 56. The method of Item 54, wherein the disease or disorder is an ischemia-related disease or disorder.
Item 57. The method of Item 56, wherein the ischemia-related disease or disorder is selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, and reperfusion injury of a transplanted organ.
Item 58. The method of Item 54, wherein the disease or disorder is a genetic disorder.
Item 59. The method of Item 54, wherein the disease or disorder is an aging disease or disorder.
Item 60. The method of Item 54, wherein the disease or disorder is a neurodegenerative condition or cardiovascular condition.
Item 61. The method of Item 60, wherein the neurodegenerative condition is selected from the group consisting of dementia, Friedrich's ataxia, amyotrophic lateral sclerosis, mitochondrial myopathy, encephalopathy, lactacidosis, stroke (MELAS), myoclonic epilepsy with ragged red fibers (MERFF), epilepsy, Parkinson's disease, Alzheimer's disease, or Huntington's Disease. Exemplary neuropsychiatric disorders include bipolar disorder, schizophrenia, depression, addiction disorders, anxiety disorders, attention deficit disorders, personality disorders, autism, and Asperger's disease.
Item 62. The method of Item 60, wherein the cardiovascular condition is selected from the group consisting of coronary heart disease, myocardial infarction, atherosclerosis, high blood pressure, cardiac arrest, cerebrovascular disease, peripheral arterial disease, rheumatic heart disease, congenital heart disease, congestive heart failure, arrhythmia, stroke, deep vein thrombosis, and pulmonary embolism.
Item 63. The method of Item 54, wherein the disease or disorder is a cancer, autoimmune disease, inflammatory disease, or fibrotic disorder.
Item 64. The method of Item 54, wherein the disease is acute respiratory distress syndrome (ARDS).
Item 65. The method of Item 54, wherein the disease or disorder is pre-eclampsia or intrauterine growth restriction (IUGR).
Item 66. The method of any one of Items 54-65, wherein the method comprises administering the population of isolated mitochondria or the composition to the subject via an intravenous, intra-arterial, intra-tracheal, subcutaneous, intramuscular, inhalation, or intrapulmonary route of administration.
Item 67. An isolated mitochondrion having intact inner and outer membranes, wherein the inner membrane comprises folded cristae, wherein the mitochondrion has been isolated from a cell, wherein the mitochondrion is polarized as measured by a fluorescence indicator, and wherein the mitochondrion is capable of maintaining polarization in an extracellular environment.
Item 68. The isolated mitochondrion of Item 67, wherein the mitochondrion has a non-filamentous shape.
Item 69. The isolated mitochondrion of Item 67 or 68, wherein voltage dependent anion channels (VDAC) on the surface of the mitochondrion are associated with tubulin at the surface.
Item 70. The isolated mitochondrion of Item 67, wherein the tubulin is dimeric tubulin.
Item 71. The isolated mitochondrion of Item 70, wherein the tubulin is a heterodimer comprising α-tubulin and β-tubulin.
Item 72. The isolated mitochondrion of any one of Items 67-71, wherein the fluorescence indicator is selected from the group consisting of JC-1, tetramethylrhodamine methyl ester (TMRM), and tetramethylrhodamine ethyl ester (TMRE).
Item 73. The isolated mitochondrion of any one of Items 67-72, wherein the isolated mitochondrion exhibits decreased association with mitochondria-associated membrane (MAM) as measured by glucose regulated protein 75 (GRP75) expression.
Item 74. The isolated mitochondrion of Item 73, wherein the decreased association is a decrease of at least about 30% relative to the association with MAM of a mitochondrion in a cell or of an isolated mitochondrion obtained by a method comprising homogenization of cells.
Item 75. The isolated mitochondrion of Item 74, wherein the decreased association is a decrease of at least about 50%.
Item 76. The isolated mitochondrion of any one of Items 67-75, wherein the membrane potential of the isolated mitochondrion is between about −30 mV and about −220 mV.
Item 77. The isolated mitochondrion of any one of Items 67-76, wherein the isolated mitochondrion is not undergoing drp1 dependent division.
Item 78. The isolated mitochondrion of any one of Items 67-77, wherein the isolated mitochondrion is between about 500 nm and about 3500 nm in size.
Item 79. A composition comprising the isolated mitochondrion of any one of Items 67-78.
Item 80. The population of isolated mitochondria of any one of Items 1-30, wherein the isolated mitochondria are derived or isolated from a cell whose mitochondria have been treated with a mitochondria activating agent.
Item 81. The method of any one of Items 33-52, wherein treating mitochondria in cells with a mitochondria activating agent prior to (i) to (iv).
Item 82. The composition of Item 30 or 79 or the formulation of Item 31, wherein the isolated mitochondria are derived or isolated from a cell whose mitochondria have been treated with a mitochondria activating agent.
Item 83. Isolated mitochondria, wherein the isolated mitochondria are derived or isolated from a cell whose mitochondria have been treated with a mitochondria activating agent.
Item 84. The isolated mitochondria of any one of Items 67-78, wherein the isolated mitochondria are derived or isolated from a cell whose mitochondria have been treated with a mitochondria activating agent.
Item 85. The method of any one of Items 53-66, wherein the population of isolated mitochondria to be administered is the population of isolated mitochondria of Item 80, the composition of Item 82, or formulation of Item 82.
Item 86. The population of isolated mitochondria of Item 80, the composition or formulation of Item 82, or the isolated mitochondria of Item 83 or 84, further comprising the mitochondria activating agent.
Item 87. The population of isolated mitochondria of Item 80, the composition or formulation of Item 82, or the isolated mitochondria of Item 83 or 84, wherein the mitochondria activating agent is resveratrol.
Item 88. The population, the composition, the formulation, or the isolated mitochondria of Item 86, wherein the mitochondria activating agent is resveratrol.
Item 89. Isolated mitochondria, wherein the isolated mitochondria are derived or isolated from a cell that has been treated with a lipid membrane-based vesicle encapsulating or containing a mitochondria activating agent.
Item 90. The isolated mitochondria of Item 89, wherein the mitochondria activating agent is resveratrol.
Item 91. The isolated mitochondria of Item 89 or 90, further comprising the mitochondria activating agent.
Item 92. A population of the isolated mitochondria of any one of Items 88 to 90.
Item 93. A composition comprising the population of the isolated mitochondria of any one of Items 88 to 90.
Item 94. A pharmaceutical composition comprising the population of the isolated mitochondria of any one of Items 88 to 90.
Item 95. The method of any one of Items 33 to 66, wherein the cells have been treated with a lipid membrane based vesicle encapsulating or containing a mitochondria activating agent.
Item 96. The method of Item 95, wherein the mitochondria activating agent is resveratrol.

In an embodiment, the encapsulated mitochondria can be prepared from the isolated mitochondria as explained above.

### EXAMPLES

### Example 1

### Isolation of Mitochondria

Mitochondria were isolated from HeLa cells as follows. Human-derived HeLa cells (RCB3680), which were purchased from the cell bank of Riken, were cultured. The medium used in the culture was MEM+10% FBS and subculture was carried out once or twice per week.

1) Cells were cultured in a dish of 100 mm in diameter and 80% confluence was confirmed.

2) The culture medium was discharged and the dish was washed twice with 3 mL of an isolation buffer (10 mM Tris-HCl, 250 mM sucrose, 0.5 mM EGTA, pH 7.4).

3) 3 mL of a 30 μM digitonin-comprising isolation buffer was added to the dish and the dish was allowed to stand still at room temperature for 3 minutes. 30 μM is about 1/10 of the critical micelle concentration (cmc) of digitonin.

4) The interior of the dish was washed twice with 3 mL of the isolation buffer.

5) 3 mL of the isolation buffer was added to the dish and the dish was allowed to stand still at 4° C. for 10 minutes.

6) Cells were detached by gentle pipetting by use of a micro pipette.

7) The suspension comprising mitochondria and the cells detached was then transferred to 15 mL-centrifuge tubes and centrifuged at 500×g and 4° C. for 10 minutes. The supernatant (2 mL) was collected to obtain an isolated mitochondrial population (hereinafter referred to also as a “product to be prepared at time of use”).

8) When frozen, glycerol was added not to the isolation buffer but to a freezing buffer (10 mM Tris-HCl, 225 mM mannitol, 75 mM sucrose, 0.5 mM EGTA, pH 7.4) so as to obtain a concentration of 10% and suspended. The suspension was frozen in liquid nitrogen to obtain frozen isolated mitochondria (hereinafter referred to also as “frozen product”).

The product to be prepared at time of use was stained with 250 nM tetramethylrhodamine methyl ester (TMRM) in the presence of malic acid and glutamine (each 5 mM) and the activity of the isolated mitochondria was evaluated. As a result, polarization of mitochondria was confirmed. The isolated mitochondria were stained with 100 nM of MitoTrackerDeep red and the purity thereof was checked. As a result, clear green fluorescence was emitted from almost the entire solution comprising isolated mitochondria. From this, it was confirmed that isolated mitochondria are present in almost the entire solution. In other words, isolated mitochondria were present in the ratio of 98% or more of the mitochondrial solution and 90% of the mitochondria showed polarization.

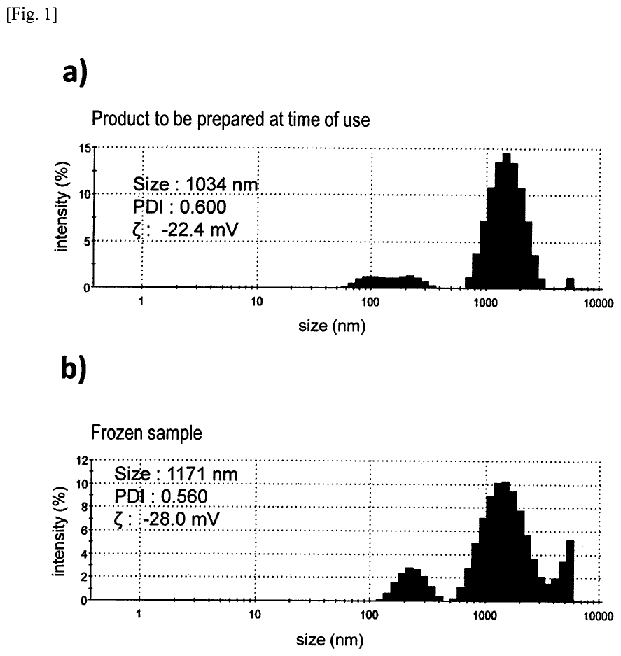

Frozen mitochondria were thawed by exposing them to miming water and centrifuged at 500×g and 4° C. for 10 minutes. The supernatant was collected followed by centrifugation to precipitate mitochondria. The supernatant was discarded and Tris buffer was added in place to obtain samples. The particle size (by dynamic light scattering) and zeta potential (by electrophoretic light scattering) of mitochondria contained in these samples were measured by Zetasizer Nano ZS (Malvern Instruments, Ltd., Worcestershire, UK). More specifically, the average particle size (average hydrodynamic particle size) and polydispersity index (PDI) of mitochondria were obtained from an autocorrelation function of scattering light intensity in accordance with the Cumulant analysis (ISO22412). Thereafter, based on the particle sizes, histograms were prepared. The results were as shown in FIG. 1. In FIG. 1, the results of the product to be prepared at time of use prior to freezing and obtained in step 7) above are shown in panel a); whereas, the results of samples obtained by thawing the frozen products obtained in step 8) above are shown in panel b). As shown in FIG. 1, particle sizes of mitochondria in the isolated mitochondrial population were distributed around about 1,000 nm (see, FIG. 1, panel a)). The same distribution result was obtained in the isolated mitochondrial population frozen and thawed (see, FIG. 1, panel b)). Furthermore, after the freeze-thaw process, the zeta potential was maintained at a negative value (see, zeta (ζ) potential of FIG. 1).

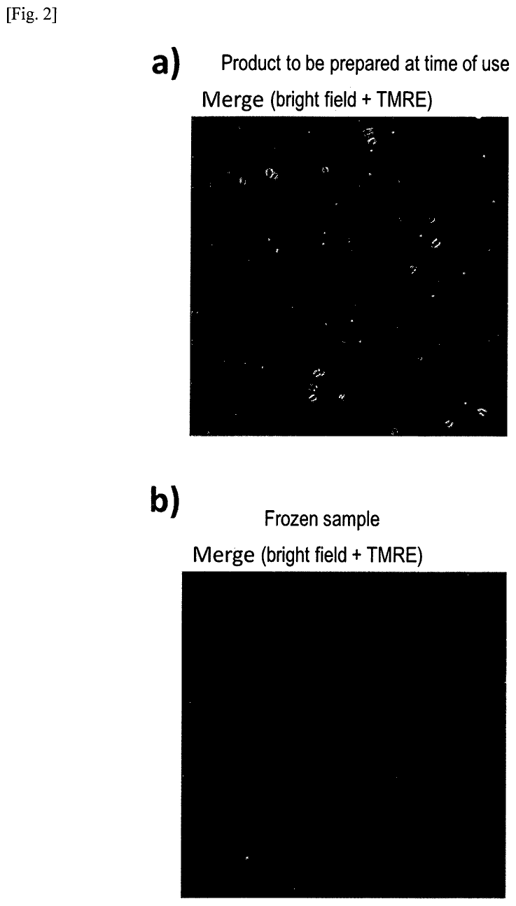

Subsequently, in order to evaluate the surface potential of mitochondria contained in the isolated mitochondrial population, observation was carried out by a confocal laser microscope. More specifically, for evaluating the membrane potential, a mitochondrial potential-dependent reagent, tetramethylrhodamine ethyl ether (TMRE)(excitation wavelength: 549 nm, fluorescence wavelength: 574 nm)(Thermo Fisher, Waltham, Mass.) was used. TMRE emits red fluorescence, if a mitochondrial membrane potential is maintained. The isolated mitochondrial solution (300 μL) was added in a 3.5 cm glass base dish (AGC TECHNO GLASS Co., LTD. (IWAKI), Shizuoka, Japan) and centrifuged at 10×g and 4° C. for 10 minutes. The supernatant was discarded. A staining solution was added so as to obtain 10 nM TMRE, 0.33 mg/mL bovine serum albumin (BSA) (Sigma-Aldrich, St. Louis, Mo.), 5 mM malic acid (Wako Pure Chemical Industries, Ltd., Osaka, Japan) and 5 mM glutamic acid (Wako Pure Chemical Industries, Ltd., Osaka, Japan) each in terms of final concentration. Incubation was carried out at room temperature for 10 minutes. Observation was carried out by use of FV10i-LVI (Olympus Corporation, Tokyo, Japan). The results are as shown in FIG. 2. In FIG. 2, the results of the isolated mitochondrial population (i.e., isolated mitochondrial population before a freeze-thaw process, hereinafter referred to also as a “product to be prepared at time of use”) obtained in step 7) above are shown in panel a); whereas, the results of a sample obtained by thawing a frozen material (i.e., isolated mitochondrial population after a freeze-thaw process, hereinafter referred to also as “frozen product”) obtained in step 8) above are shown in panel b). As shown in FIG. 2, also in the frozen product, red fluorescence is satisfactorily detected. It is demonstrated that the membrane potential is maintained even after the freeze-thaw process.

### Example 2

Coating with Lipid Membrane and Characteristic Analysis of the Particles Obtained

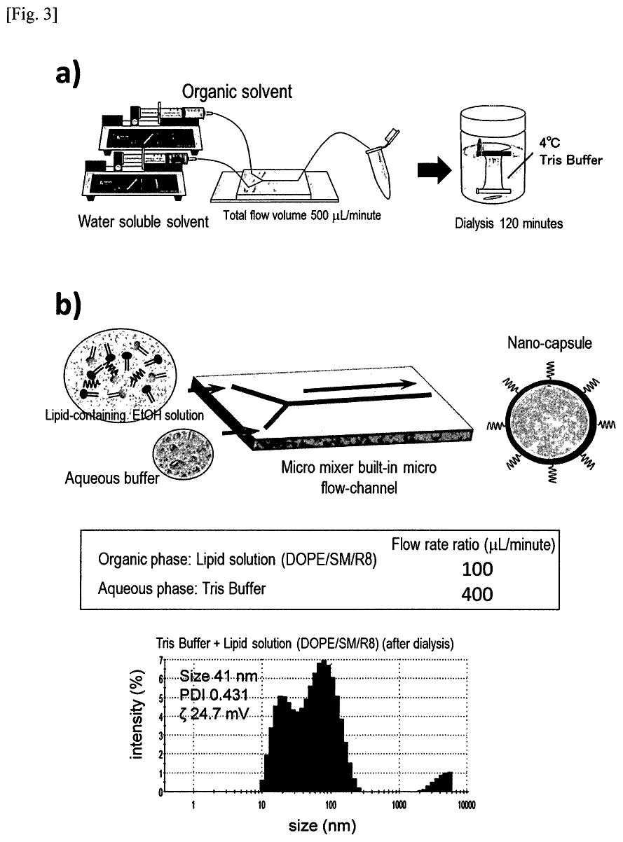

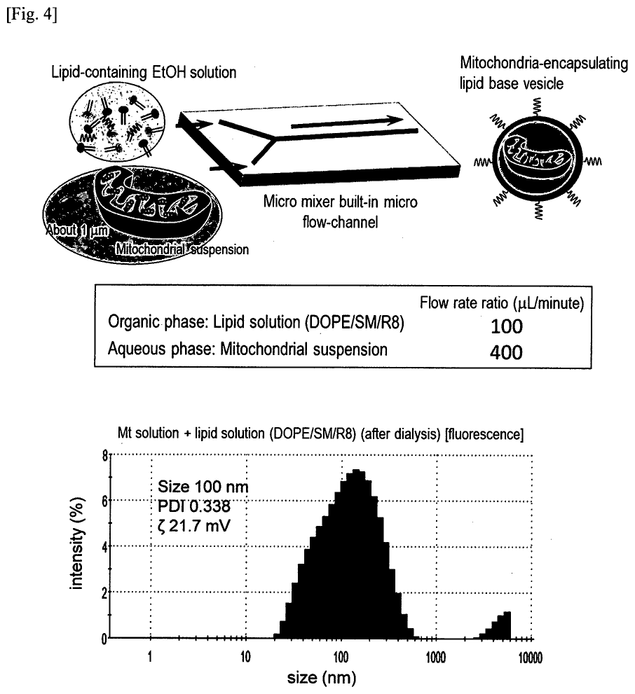

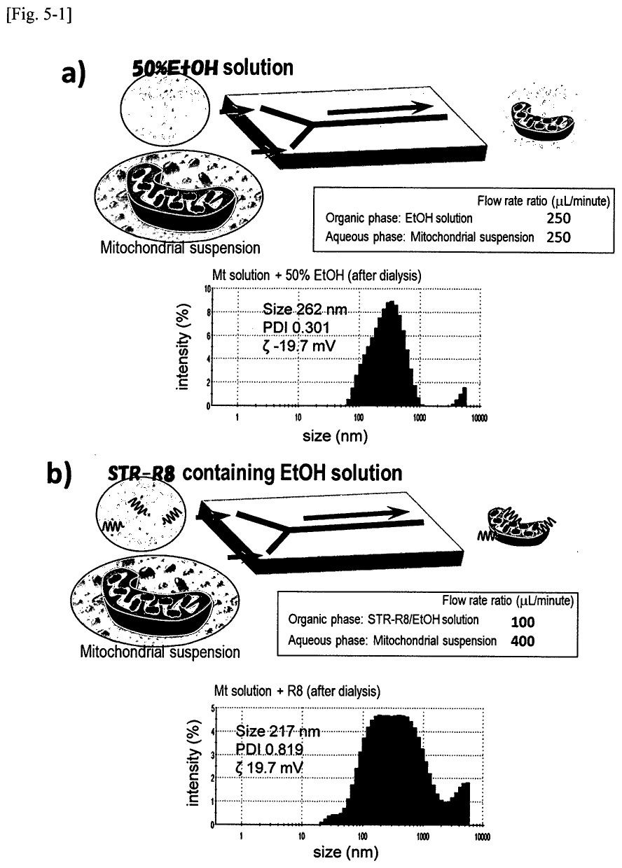

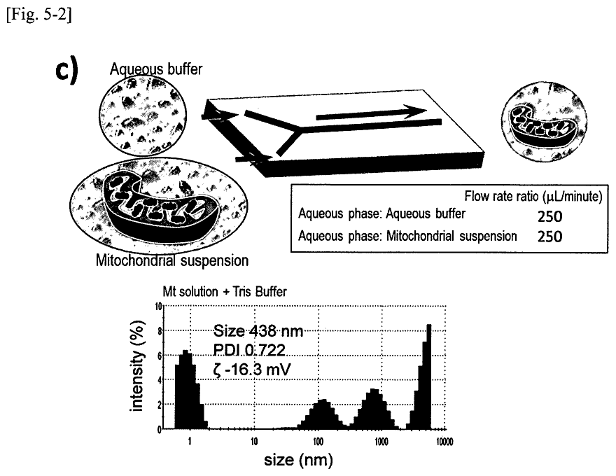

Mitochondria contained in the isolated mitochondrial population were each coated with lipid membrane (to obtain mitochondria encapsulated in lipid membrane) having a lamella structure which serves as a border topologically separating between the interior and the exterior. In coating with lipid membrane, whether a micro flow channel can be used or not was examined. The processes were specifically as follows. As the micro flow channel device, iLiNP™ (Lilac pharma Inc.) having a baffle construct was used. The device, iLiNP has, as shown in FIG. 36, two solution inlets (11a and 12a), channels (11 and 12) respectively connecting the solution inlets to a confluent channel (13) and a mixing channel (14). The confluent channel 13 is a site at which the channels (11 and 12) extending from the two solution inlets are joined. The mixing channel 14 is a channel at which the joined solutions are mixed. As shown in FIG. 36, in the mixing channel 14, the solution moves along with the flow direction indicated by a big arrow and is guided toward an outlet 14c via bends. The mixing channel 14 had a single or a plurality of sets (20 sets) of bends represented by 14a and 14b. In FIG. 36, 14a represents the region at which the width of the channel is narrowed (50 μm in width) and 14b (200 μm in width) represents the region at which the width of the narrowed region is returned to the original width. The height of the channel was 100 μm. According to the micro flow channel device, an organic solvent and a water-soluble solvent can be stirred at a high speed (see, FIG. 3, panel a)). In the Example, the organic phase used herein was 7.7 mM lipid solution (1,2-dioleoyl-sn-glycero phosphoethanolamine (DOPE)/sphingomyelin (SM)/1,2-dimrystoyl-sn-glycerol, methoxy polyethylene glycol 2000 (DMG-PEG 2000)/stearylated octaarginine (STR-R8)=9/2/0.33/1.1 (molar ratio)) dissolved in ethanol; and the aqueous phase used herein was a solution of isolated mitochondria. These two solutions were respectively applied to the two inlets of the micro flow channel device and mixed in the confluent channel of the micro flow channel device (see, FIG. 4). The mixing conditions were as follows: total flow rate: 500 μL/minute (organic phase: 100 μL/minute, aqueous phase: 400 μL/minute). The syringe pump used herein was PUMP 11 ELITE (Harvard Apparatus, Holliston, Mass.). As the controls, a sample comprising no isolated mitochondria in the aqueous phase (see, FIG. 3, panel b)), a sample using ethanol alone in the organic phase (see, FIG. 5, panel a)) and a sample using ethanol comprising STR-R8 alone in the organic phase were evaluated (see, FIG. 5, panel b)) were evaluated. Further, a sample using a mixture of a water-soluble solvent and isolated mitochondrial solution in place of the organic phase, was also evaluated (see, FIG. 5, panel c)). The particle sizes and zeta potentials of individual samples were measured. As a result, as shown in FIG. 4, the particles obtained from the micro flow channel device showed a monodispersed particle size distribution having a peak around 100 nm. Since mitochondria having an activity is polarized, they have a negative zeta potential. In contrast, the particles obtained by a micro flow channel device showed a positive zeta potential (see, FIG. 4). This fact suggests that mitochondria were encapsulated by lipid membrane exposing cationic STR-R8 on the surface, and that the zeta potential was changed to being positive due to the presence of said R8.

Further, it was shown that the mitochondria have been divided into smaller ones (size: 262 nm; PDI: 0.301; and ζ potential: −19.7 mV), and have no detectable negative membrane polarization after the division with the micro flow channel by using a mitochondrial membrane potential indicator.

### Example 3

### Observation of Mitochondria-Encapsulating Lipid Membrane-Based Vesicles by Fluorescence Microscope

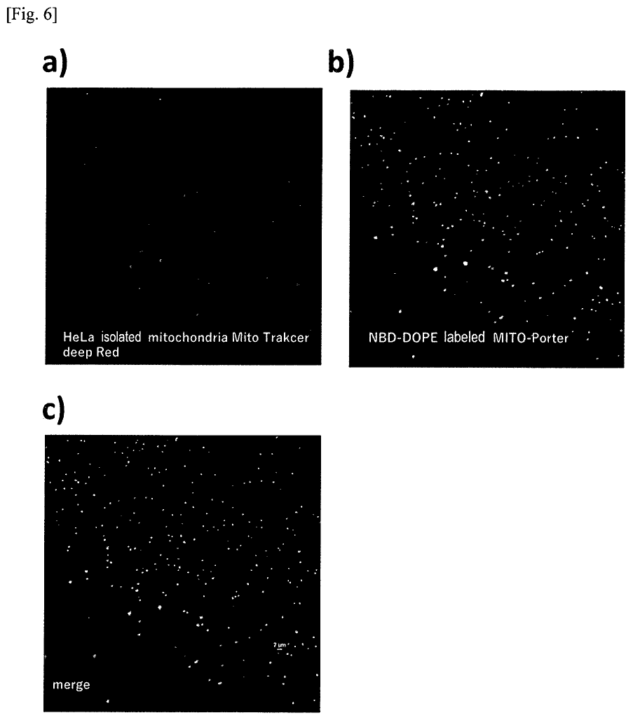

Next, in order to determine whether mitochondria were successfully coated with lipid membrane or not, mitochondria were stained in red (MitoTracker (trademark) Deep Red (excitation wavelength: 644 nm, fluorescence wavelength: 665 nm) (Thermo Fisher, Waltham, Mass.)); whereas, lipid was stained in green (DOPE-N-(7-nitro-2-1,3-benzoxadiazol-4-yl) (NBD-DOPE) (excitation wavelength: 465 nm, fluorescence wavelength: 535 nm) (Avanti Polar lipids, Alabaster, Ala.), and then, the isolated mitochondria were subjected to a coating process using the micro flow channel device as described above, and observed by a fluorescence imaging. If the mitochondria were coated, red and green overlap and yellow fluorescence should be generated. The particles obtained by the micro flow channel device were added to a glass slide and observed by Nikon Al (Nikon Corporation, Tokyo, Japan). The results were as shown in FIG. 6. As shown in FIG. 6, red fluorescence showing the presence of mitochondria (see, FIG. 6, panel a)) and green fluorescence showing the presence of lipid (see, FIG. 6, panel b)) were completely co-present (see, FIG. 6, panel c)). The results support that the obtained particles are particles in the form of mitochondria encapsulated by lipid membrane.

### Example 4

### Observation of Mitochondria-Encapsulating Lipid Membrane-Based Vesicles by Electron Microscope

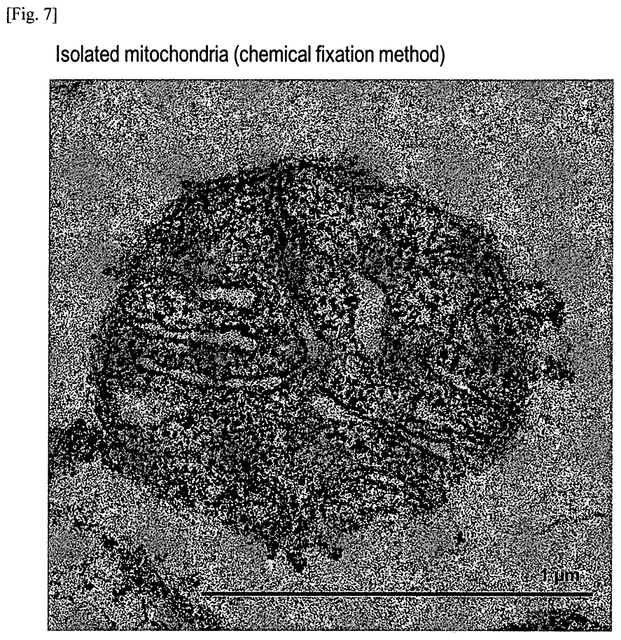

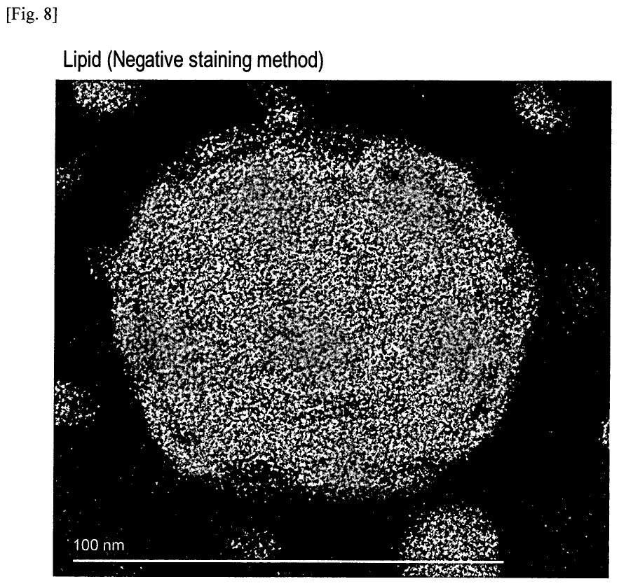

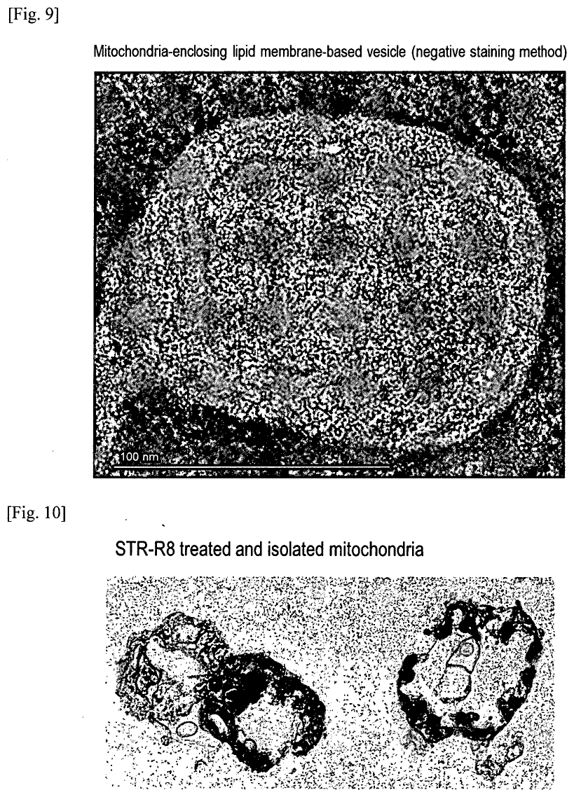

The structure of the mitochondria-encapsulating lipid membrane-based vesicles (see, FIG. 4) obtained by the above micro flow channel device was observed by an electron microscope. The isolated mitochondria (product to be prepared at time of use) and isolated mitochondria with STR-R8 modification (mitochondria shown in FIG. 5, panel b)) were used as controls. More specifically, the isolated mitochondria and isolated mitochondria with STR-R8 modification were stained in accordance with a chemical fixation method, which are routinely employed for observing biological samples. Simple lipid membrane-based vesicles (see, FIG. 3, panel b)) and mitochondria-encapsulating lipid membrane-based vesicles (see, FIG. 4) were stained in accordance with a negative staining method suitable for nanoparticle observation. The chemical fixation method was carried out as follows. First, a sample was fixed with the same amount of a 0.1 M cacodylate buffer (pH 7.4) comprising 4% of paraformaldehyde and 2% of glutaraldehyde, cooled, and thereafter further fixed with a 0.1 M cacodylate buffer (pH 7.4) comprising 2% of glutaraldehyde at 4° C., overnight. The sample fixed was washed with the cacodylate buffer, and then further fixed with a 0.1 M cacodylate buffer (pH 7.4) comprising 2% of osmium tetraoxide. The sample was soaked stepwise in a solution comprising ethanol to remove water. The sample was treated twice with propylene oxide and incubated in a mixture comprising propylene oxide and a resin (Quetol-812; Nisshin EM Co., Tokyo, Japan) in a ratio of 70:30 for one hour. Thereafter, the cap of a tube was removed and the sample was allowed to stand still overnight to evaporate propylene oxide. Thereafter, the sample was embedded in 100% resin and a polymerization reaction was carried out at 60° C. for 48 hours. The sample was sliced into sections of 70 nm in thickness. The sections were observed by an electron microscope. Nanoparticles were observed by a negative staining method. This is because lipid membrane is decomposed by the chemical fixation method and the resultant sample is not suitable for observing a membrane structure. The electron microscope used herein was JEM-1400 Plus (JEOL Ltd., Tokyo, Japan). Observational analysis was outsourced to Tokai Electron Microscopy, Inc. The results were as shown in FIGS. 7 to 10. In the chemical fixation method as shown in FIG. 7, it was observed that the isolated mitochondria have a christie structure characteristic in mitochondria. As shown in FIG. 8, it was observed that the lipid-membrane base vesicle not encapsulating mitochondria has a particle form; however the interior of the particle was filled up with lipid membrane. In contrast, it was observed that the mitochondria-encapsulating lipid membrane based vesicle (see, FIG. 4) has a hollow lipid membrane structure, as shown in FIG. 9. The lipid membrane, if no material is encapsulated inside, the interior of a particle is filled up with lipid membrane as shown in FIG. 8. In the particle shown in FIG. 9, since no lipid membrane was able to enter the interior, it was presumed that mitochondria are encapsulated. Note that, the negative staining method is not an appropriate method for observing the mitochondrial structure as shown in FIG. 7. Because of this, mitochondria were not shown in FIG. 9. However, in STR-R8 modified and isolated mitochondria, it was observed that mitochondrial structure is destroyed as shown in FIG. 10. It was found that mitochondria are preferably modified with STR-R8 after the mitochondria were encapsulated by lipid membrane.

### Example 5

### Mitochondria-Encapsulating Lipid Membrane-Based Vesicles Using Various Types of Lipids

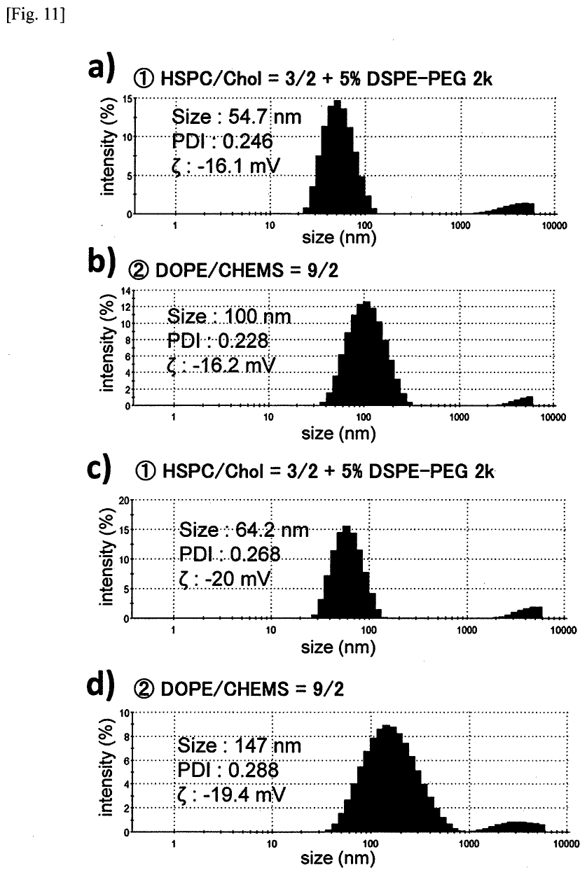

In place of the lipid membrane composition (nano-capsule material: DOPE/SM/STR-R8) of mitochondria-encapsulating lipid membrane-based vesicles prepared in FIG. 4, various lipid membrane compositions were used to examine whether or not mitochondria-encapsulating lipid membrane-based vesicles can be prepared from these compositions. The lipid membrane material compositions used herein were a neutral lipid membrane composition: hydrogenated soybean phosphatidylcholine (HSPC)/cholesterol (Chol)/1,2-dimyristoyl-rac-glycero-3-methoxy polyethylene glycol-2000 (DMG-PEG 2000)=3/2/0.25 (molar ratio), which is a component of the same composition as that of nano-capsule clinically used, Doxil; and DOPE/cholesterol hemisuccinate (CHEMS)=9/2 (molar ratio), which is a component of a lipid membrane composition having a negative potential. The mitochondria-encapsulating lipid membrane-based vesicles were prepared by the micro flow channel device as mentioned above. As a negative control, particles prepared from a solution not comprising mitochondria and an organic solvent comprising a lipid, were used. The results were as shown in FIG. 11. As shown in FIG. 11, it was observed that any one of particle groups shows a particle size distribution without aggregation. It was found that the particle size of the mitochondria-encapsulating lipid membrane based vesicles (“mitochondria packaging” in the figure) tends to be bigger than that of the particles having no mitochondria encapsulated therein (“nanoparticles” in the figure). It was also found that, in not only the composition positively charged and shown in FIG. 4 but also both neutral lipid (see, FIG. 11, panel c)) and lipid negatively charged (see, FIG. 11, panel d)), mitochondria-encapsulating lipid membrane-based vesicles can be obtained. Based on the comparison between particle sizes, the possibility that particle size distribution changes depending on the lipid membrane composition, was suggested (see, FIG. 11).

### Example 6

### Mitochondria-Encapsulating Lipid Membrane-Based Vesicles Packaged at Various Flow Rates

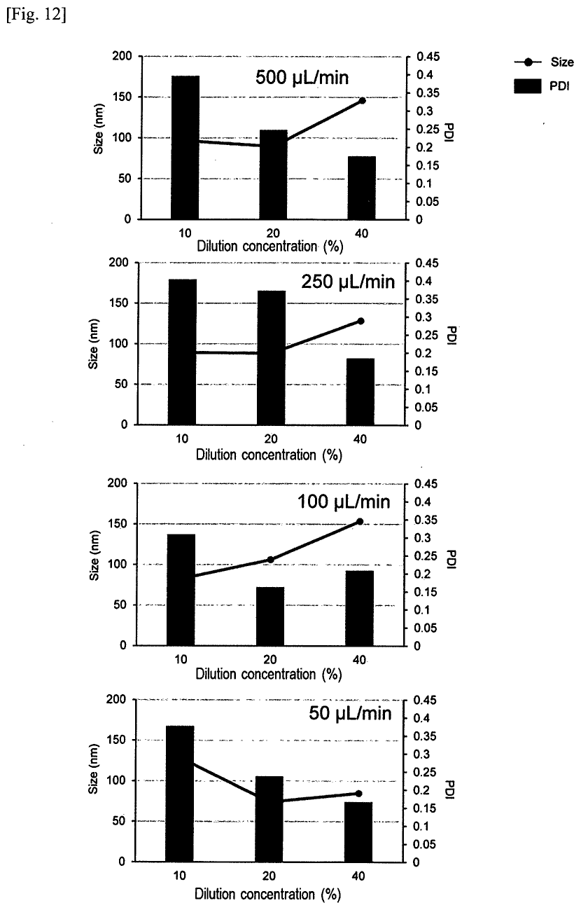

Mitochondria-encapsulating lipid membrane-based vesicles were prepared by using the same micro flow channel device as in the above (see, FIG. 4) except that only the flow rate and ethanol concentration of the organic phase were changed. The total flow rate was changed to be 50 μL/minute, 100 μL/minute, 250 μL/minute, or 500 μL/minute. The ethanol concentration of the organic phase was changed to be 10%, 20% or 40%. The particle sizes and zeta potentials of the obtained mitochondria-encapsulating lipid membrane-based vesicles were measured in the same manner as above. The results were as described in FIG. 12. As described in FIG. 12, in any conditions, the particle size was about 100 to 150 nm.

### Example 7

Incorporation of the Obtained Mitochondria-Encapsulating Lipid Membrane-Based Vesicles into Cells

Incorporation of the mitochondria-encapsulating lipid membrane-based vesicles obtained in FIG. 4 into cells and intracellular dynamics thereof in the cells after incorporation were observed. Mitochondria in the mitochondria-encapsulating lipid membrane-based vesicles were stained in red with MitoTracker (trademark) Deep Red before packaging of isolated mitochondria (by incubating them at a concentration of 100 nM at 4° C. for 15 minutes for the purpose of staining the mitochondria). HeLa cells were prepared and mitochondria in the HeLa cells were stained in green (by incubating them at a concentration of 100 nM and 37.0° C., in 5% CO₂ condition for 15 minutes) with MitoTracker (trademark) Green. Thereafter, to the resultant HeLa cells, the mitochondria-encapsulating lipid membrane-based vesicles were added. The mixture was incubated for 3 hours. After incubation, the cells were observed by a confocal laser scanning microscope (CLSM, machine used OlympusFV10i-LIV, objective lens UPlanSApo 60×/NA=1.2 water, LD laser 473 nm, 635 nm). The results were as shown in FIG. 13. As shown in FIG. 13, in the HeLa cells, a red signal of the mitochondria derived from the mitochondria-encapsulating lipid membrane-based vesicles and a green signal of the mitochondria derived from HeLa cells were observed. Almost all mitochondria showed co-localization. From this, it was found that the mitochondria derived from the mitochondria-encapsulating lipid membrane-based vesicles were incorporated into cells; and the mitochondria incorporated into cells are fused with mitochondria in the cells; and that fusion are uniformly carried out.

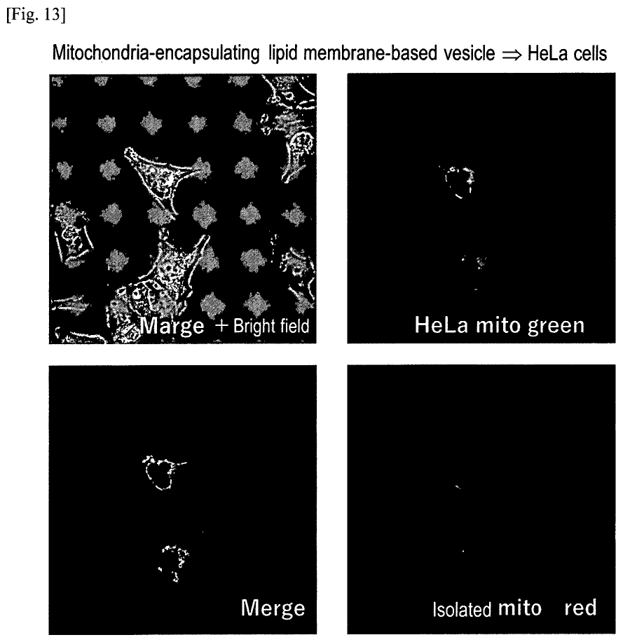

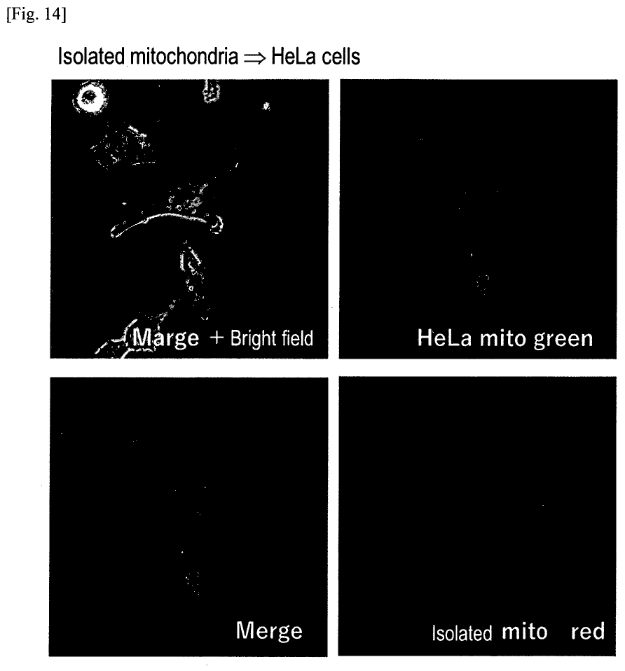

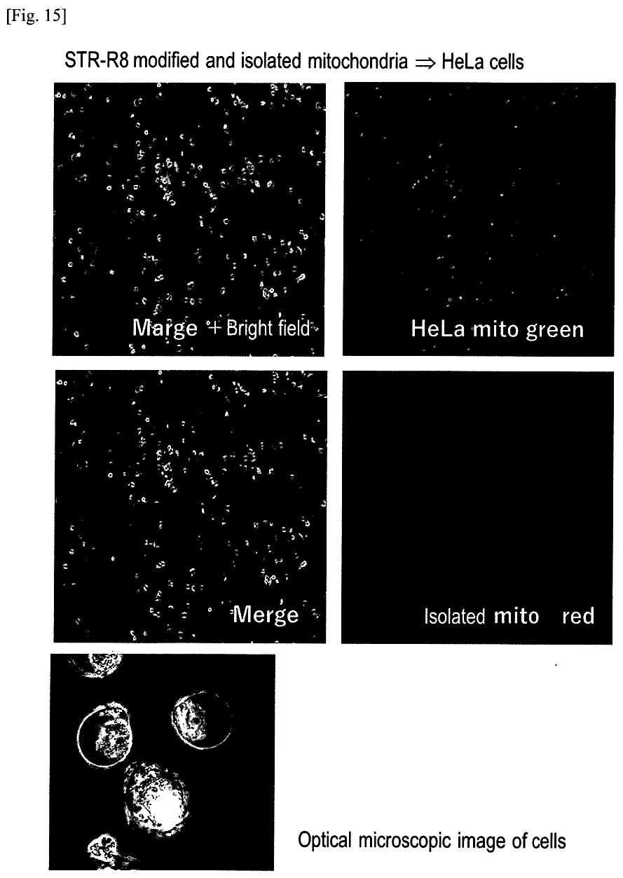

In contrast, in the case where isolated mitochondria (the same amount) before packaging were added to HeLa cells, red signal was not virtually observed in the cells (see, FIG. 14). It was observed that in the HeLa cells, to which STR-R8 modified and isolated mitochondria (not coated with a lipid) were added, many cells died (see, FIG. 15).

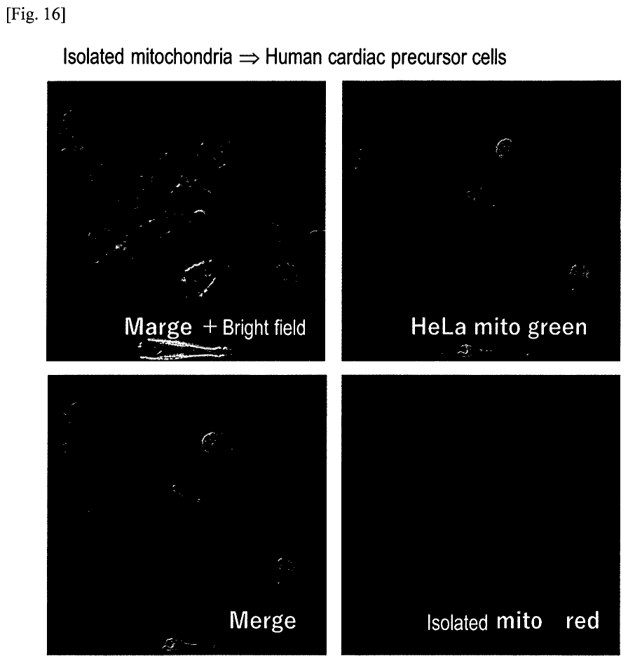

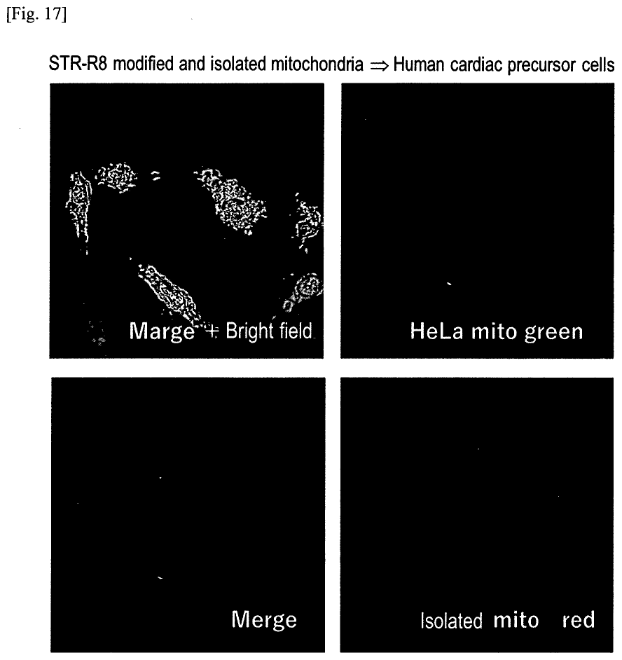

Mitochondria were isolated from human cardiac muscle stem cells (hCDC) and brought into contact with HeLa cells in the same manner as above. Thirty minutes later, incorporation of the isolated mitochondria into the cells was observed. Incorporation of the isolated mitochondria not packaged with lipid into the cells was not observed (see, FIGS. 16 and 17).

### Example 8

### Rescue Experiment of Mitochondrial Disease Model Cells

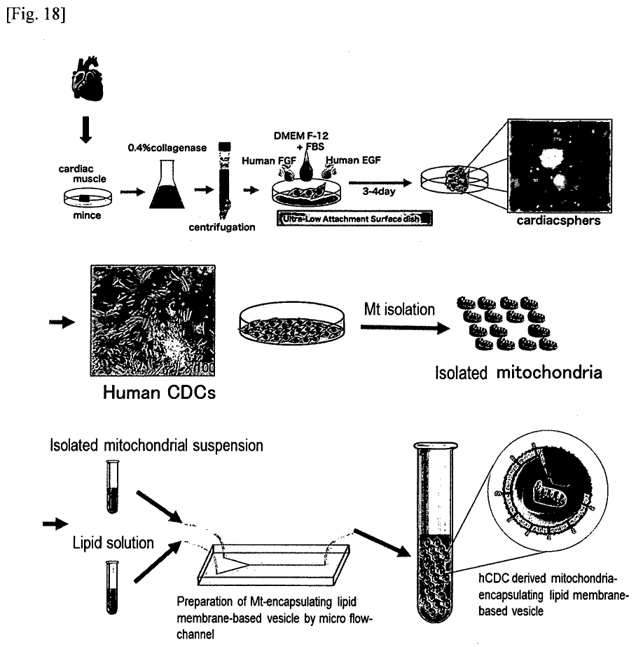

In the experiment, mitochondria were isolated from human cardiac muscle stem cells (hCDC), packaged in lipid and transplanted into cells having a mitochondrial mutation. In this manner, mitochondrial mutation was rescued. Isolated mitochondrial population was collected from hCDC in the same manner as above except that the cells were changed to hCDC (see, FIG. 18). The collected population of isolated mitochondria was packaged in lipid membrane as shown in FIG. 4 to obtain lipid membrane-based vesicles encapsulating hCDC-derived mitochondria. Thereafter, the obtained lipid membrane-based vesicles encapsulating hCDC-derived mitochondria were added to skin fibroblasts obtained by isolation culture from a MELAS patient (MELA cell), and skin fibroblasts obtained by isolation culture from a LHON patient (LHON cell). Three hours and 24 hours later, the mitochondrial respiratory activity was evaluated by an extracellular flux analyzer (machine used: extracellular flux analyzer XFp, Agilent Technologies, California, USA). To wells of an assay plate, cells were seeded at a rate of 15,000 cells/well. Three hours and 24 hours before the assay, lipid membrane-based vesicles encapsulating hCDC-derived mitochondria, were added. To the basal medium for respiratory activity measurement, glucose (5.5 mM), pyruvate (1.25 mM) and glutamine (4.0 mM) were added. After fundamental respiration was measured by an extracellular flux analyzer, oligomycin (final concentration 1 μM, carbonylcyanide-p-trifluoromethoxyphenylhydrazone (FCCP) (final concentration 1.5 μM), and rotenone and antimycin A (final concentration each 0.5 μM) were added sequentially in this order to measure the mitochondrial oxygen consumption rate.

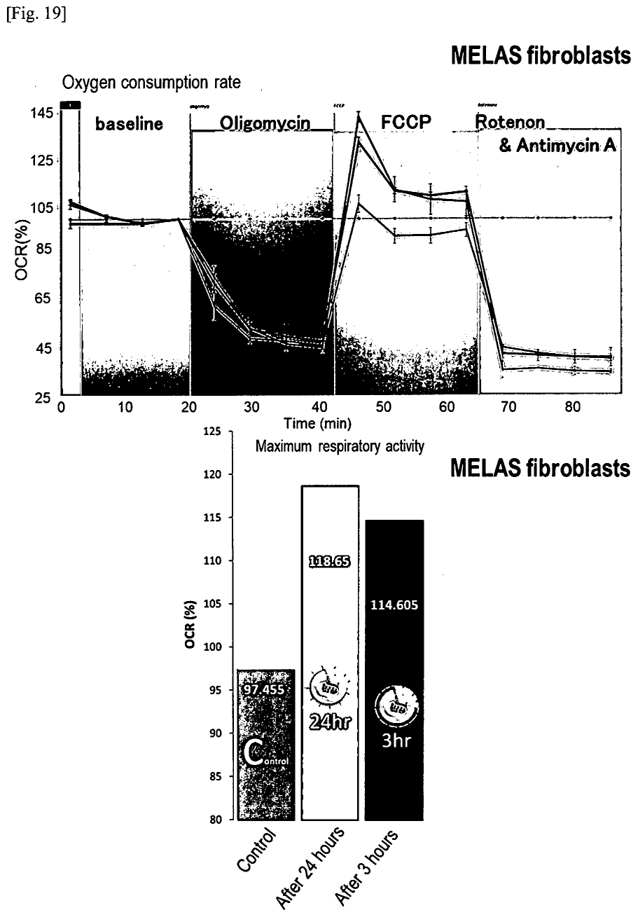

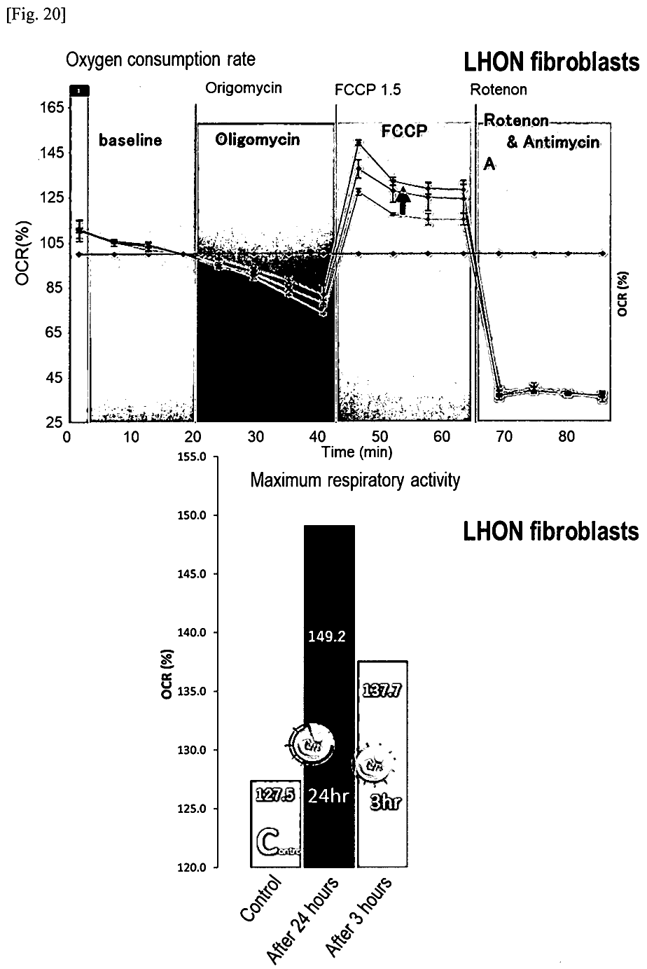

The results were as shown in FIGS. 19 and 20. As shown in FIGS. 19 and 20, mitochondrial respiratory activity after addition of FCCP was greatly improved by adding lipid membrane-based vesicles encapsulating hCDC-derived mitochondria. The improvement of the respiratory activity was observed even after 3 hours and a further great improvement thereof was observed after 24 hours. As described, it was found that mitochondrial function in the treated cells is improved by transplanting mitochondria-encapsulating lipid membrane-based vesicles into the cells. The improvement was observed within only three hours, suggesting that the mitochondrial genome DNA does not necessarily be required for the improvement, and rather, some of the other components in the encapsulated mitochondria might help to support the intracellular mitochondrial function.

### Example 9

Delivery of Mitochondria to Cells by Mitochondria-Encapsulating Lipid Membrane-Based Vesicles in Comparison with Delivery of Mitochondria to Cells by Lipofection

As the mitochondria-encapsulating lipid membrane-based vesicles, lipid membrane-based vesicles encapsulating hCDC-derived mitochondria prepared as mentioned above were used. For lipofection, the product to be prepared at time of use prepared as mentioned above and a lipid complex (lipoplex, hereinafter referred to also as “LFN iso Mt”), which was prepared by blending a solution (1 μL) of lipofectamine (Lipofectamine® 2000 Invitrogen, California, USA) with 0.32 μg (in terms of protein) of mitochondria, were used. As the cells, normal skin fibroblasts (normal fibroblasts), skin fibroblasts obtained by isolation culture from a Leigh encephalopathy patient (Leigh encephalopathy cells) and skin fibroblasts obtained by isolation culture from a LHON patient (LHON cell), were used. To the wells of an assay plate, the cells were seeded at a rate of 15,000 cells/well. Twenty-four hours before the assay, lipid membrane-based vesicles encapsulating hCDC-derived mitochondria and LFN iso Mt were added (the amounts of introduced mitochondria were the same). To the basal medium for respiratory activity measurement, glucose (5.5 mM), pyruvate (1.25 mM) and glutamine (4.0 mM) were added. After fundamental respiration was measured by an extracellular flux analyzer, oligomycin (final concentration 1 μM, FCCP (final concentration 1.5 μM), and rotenone and antimycin A (final concentration each 0.5 μM) were added sequentially in this order to measure the mitochondrial oxygen consumption rate.

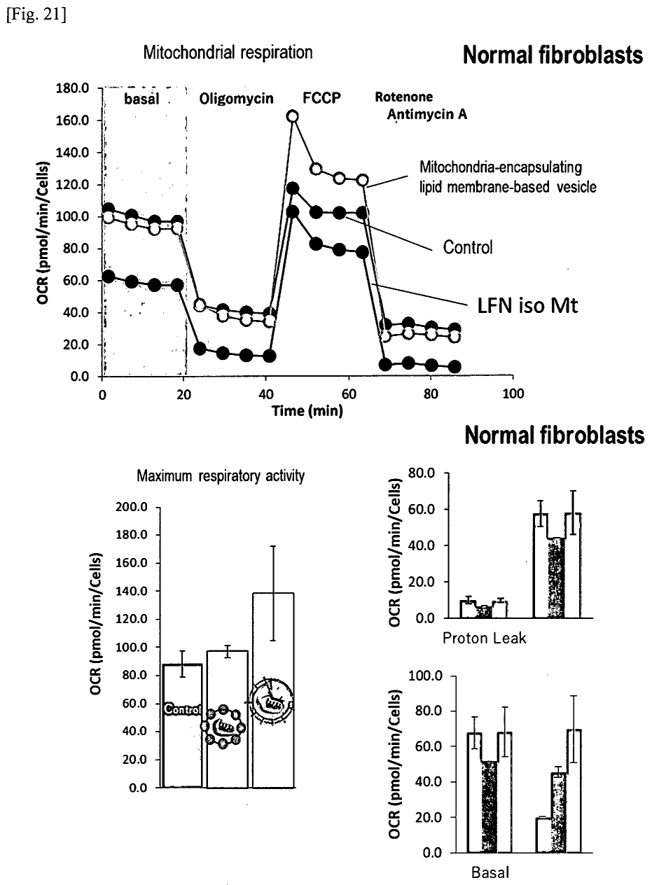

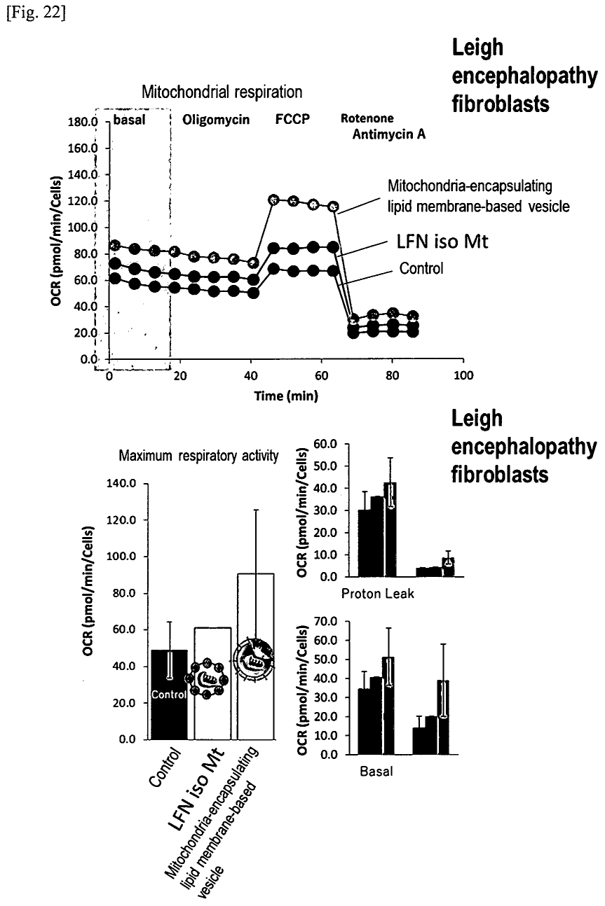

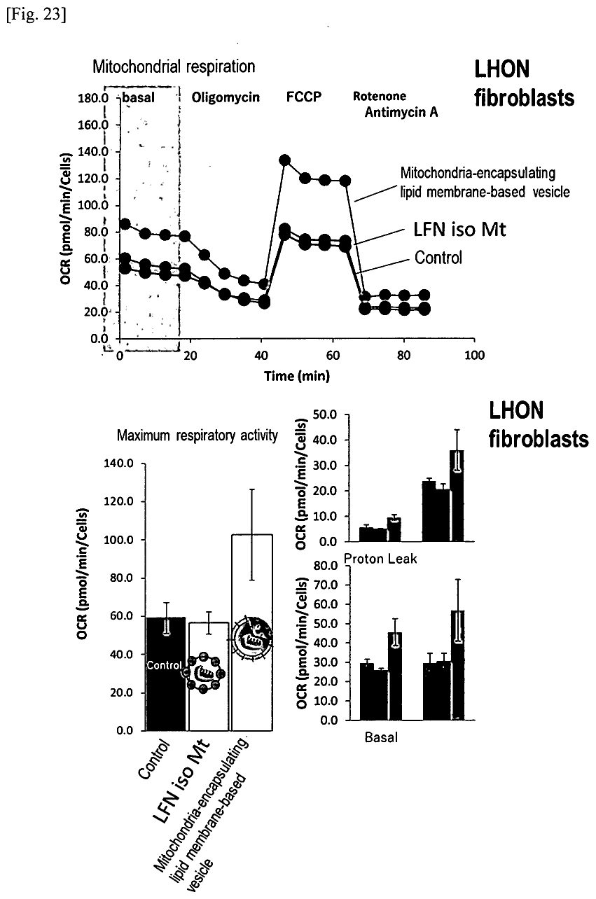

The results were as shown in FIGS. 21 to 23. As shown in FIGS. 21 to 23, the mitochondria-encapsulating lipid membrane-based vesicles increased mitochondrial respiratory activity of any one of the cells including normal cells, Leigh encephalopathy fibroblasts, and LHON fibroblasts. In contrast, in the group using lipofectamine (LFN iso Mt), an increase of mitochondrial respiratory activity was not observed or limited. The group using lipofectamine (LFN iso Mt) as show in FIG. 21 had a negative effect on the mitochondrial respiratory activity of normal cells. From this, it was suggested that the group using lipofectamine (LFN iso Mt) is possibly cytotoxic.

Subsequently, the capability of mitochondria-encapsulating lipid membrane-based vesicles to be incorporated into cells was compared to that of LFN iso Mt. Test samples were each prepared in the same manner as above except that mitochondria were taken from normal skin fibroblasts in the same manner as in the product to be prepared at time of use. The capabilities of the test samples to be incorporated into cells were evaluated by a flow cytometer (CytoFlex, Beckman Coulter, Inc., Tokyo, Japan). HeLa cells were seeded in a 6-well plate in a ratio of 2.0×10⁵ cells/well. Eight hours after seeding, a LFN iso Mt solution, an isolated mitochondrial solution, and mitochondria-encapsulating lipid membrane-based vesicles were added such that the same amount of mitochondria were supplied. Twenty-four hours later, Hela cells were collected, and the intensity of fluorescence emitted from MITO MitoTracker (trademark) Deep Red in the cells was measured by a flow cytometer (Laser 638 nm, channel APC).

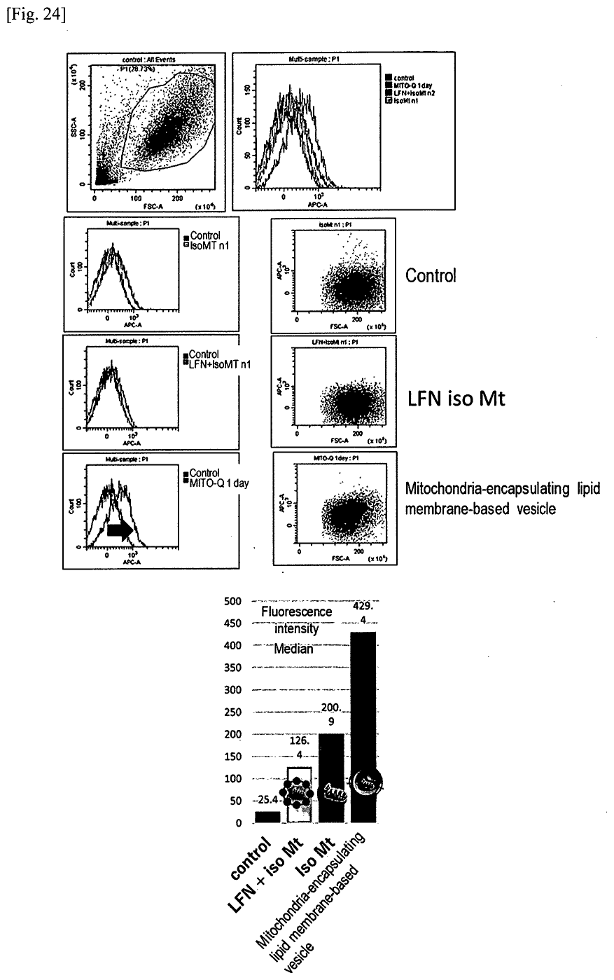

The results were as shown in FIG. 24. As shown in FIG. 24, it was confirmed that the capability of the group using lipofectamine (LFN iso Mt) to enter into cells is lower than that of isolated mitochondria. It was confirmed that the mitochondria-encapsulating lipid membrane-based vesicles are efficiently incorporated into cells similarly to those shown in the observation image of FIG. 13.

### Example 10

### Comparison Between Mitochondria-Encapsulating Lipid Membrane-Based Vesicles and Lipofectamine-Mitochondria Complex

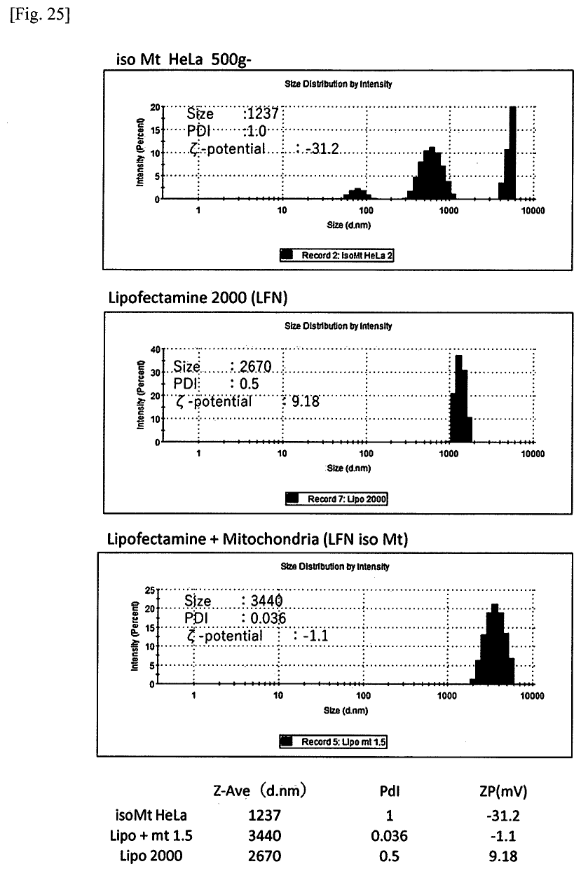

Isolated mitochondria (product to be prepared at time of use), and a mixture of a product to be prepared at time of use and lipofectamine 2000, i.e., a mixture prepared by blending 1 μL of a lipofectamine solution (Opti MEM) and mitochondria (0.32 μg in terms of protein) (LFN iso Mt) were prepared. The particle size distributions and zeta potentials of them were measured by Zetasizer. The results were as shown in FIG. 25. As shown in FIG. 25, lipofectamine had a particle size of about 2670 nm and the lipofectamine-mitochondria complex had a particle size of about 3500 nm. The lipofectamine-mitochondria complex had a small minus zeta potential. This means that the complex was electrically neutralized; in other words, that mitochondria are not completely encapsulated by lipofectamine (positive charge), and suggested that lipofectamine and free-form mitochondria may form a complex so as to neutralize charges.

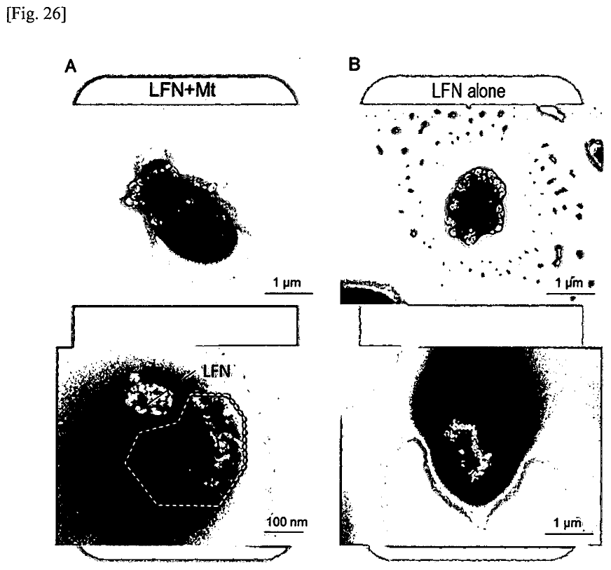

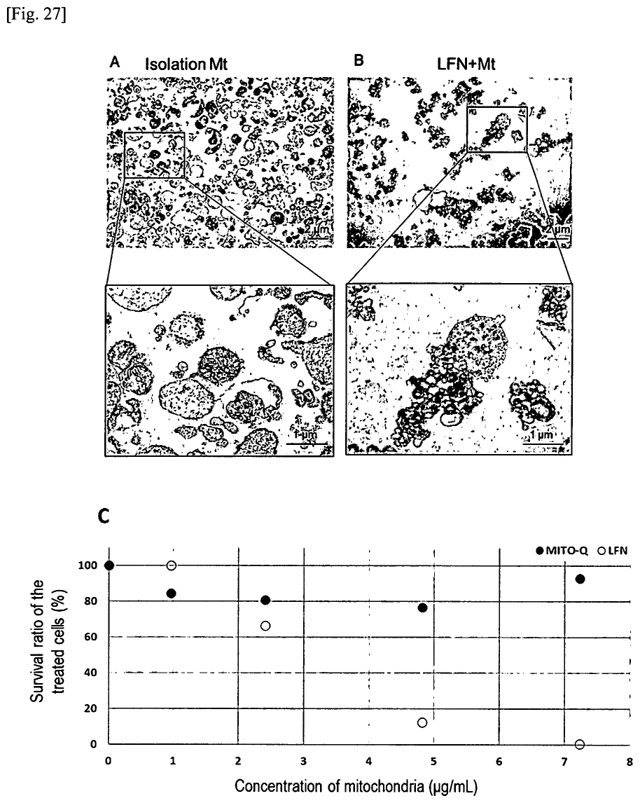

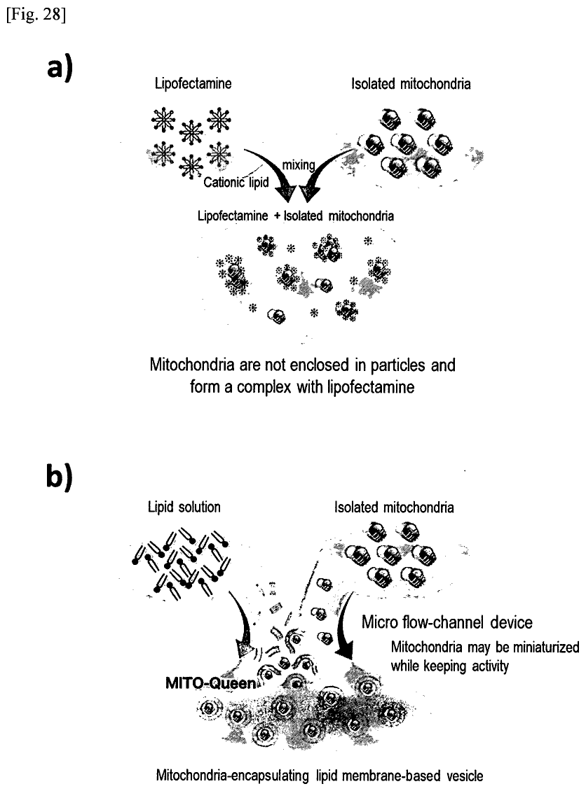

With respect to the mixture of the product to be prepared at time of use and lipofectamine 2000, a lipofectamine-mitochondria association formed in the mixture was observed by an electron microscope. The mixture of the product to be prepared at time of use and lipofectamine 2000 were stained with a chemical fixation method and a negative staining method, respectively in accordance with routine manners, and then, an association was observed. The results of the negative staining were as shown in FIG. 26 and the results of the chemical fixation method were as shown in FIG. 28. As shown in FIG. 26, lipofectamine 2000 (LFN alone) formed an association of particles in a solution. In contrast, in the case of the mixture of lipofectamine 2000 and mitochondria (LFN+Mt), an association between an association of LFN particles and debris-like mitochondria (encapsulated by white dashed line) was observed. As shown in FIG. 27, in a lipofectamine 2000-mitochondria mixture (LFN+Mt; panel B) compared to the product to be prepared at time of use (panel A), it was observed that an aggregate of lipofectamine particles are associated with a side of a part of mitochondria. Thus, the complex of LFN and mitochondria is clearly not a lipid membrane-based vesicle encapsulating mitochondrion.

Further, the cytotoxicity of MITO-Q was measured and compared to that of LFN+Mt. HeLa cells were plated at 1.0×10⁴ cells/well, and cultured for 24 hours. Then, the Hela cells were contacted with MITO-Q or Q treated with LFN at a different concentration. After one hour incubation, the culture medium was removed, and 500 μL of the fresh medium (FBS(−)) was added to each of the wells to further incubate the cells. After 24 hours of the mitochondria treatment, the cells were washed with 500 μL of PBS. Then, the incubated cells were subjected to WST-1 assay (Premix WST-1 Cell Proliferation Assay System, Takara Bio, Japan) to measure a survival rate (%) of the treated cells. The results were shown in Panel C in FIG. 27. As shown in Panel C in FIG. 27, the HeLa cells shows a cytotoxicity by the treatment with LFN+Mt in a dose dependent manner, while the Hela cells shows substantially no cytotoxicity by the treatment with MITO-Q. It is suggested that the fully encapsulated mitochondria may decrease the cytotoxicity of the mitochondria-encapsulating particle. It is also suggested that smaller mitochondria may be useful in preparing a lipid membrane-based nano vesicle without exhibiting substantial cytotoxicity.

The results were summarized in FIG. 28. As shown in FIG. 28, in the mixture of lipofectamine and isolated mitochondria, particles are formed; however, mitochondria are not encapsulated within the particles. It is considered that mitochondria (particles) and lipofectamine particles are formed into complexes. In contrast, in the present invention, lipid membrane-based vesicles encapsulating mitochondria in a closed space were obtained. The mitochondria-encapsulating lipid membrane-based vesicles have mitochondrial activity. Introduction of the vesicles into cells can enhance the mitochondria activity of the cells. In addition, since the micro flow channel device was used for mixing isolated mitochondria with a lipid solution, the isolated mitochondria were segmented into small populations while keeping the activity and encapsulated in lipid membrane-based vesicles in the state of segmented small populations. According to the present invention, not only mitochondria-encapsulating lipid membrane-based vesicles are provided but also the mitochondria-encapsulating lipid membrane-based vesicles can be miniaturized. The miniaturized mitochondria-encapsulating lipid membrane-based vesicles can be also provided.

### Example 11

### Effect of Loss of Membrane Potential on Improving Intracellular Mitochondria Function

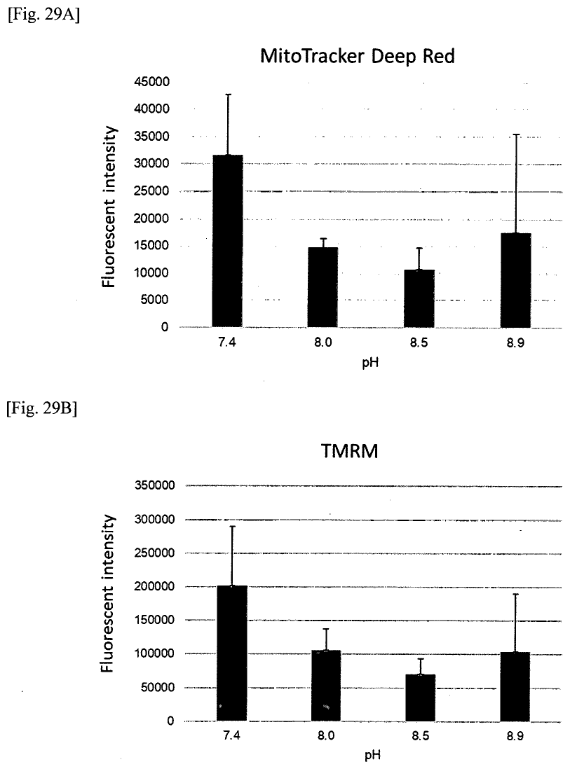

The isolated mitochondria were prepared as shown in Example 1, except that a solution having pH 7.4 to 8.9 was used throughout the isolation process, as indicated in FIGS. 29A and 29B. Then, the mitochondria isolated in a solution having a different pH were stained with 100 nM MitoTracherDeep Red or 250 nM tetramethylrhodamine methyl ester (TMRM) in the presence of malic acid and glutamine (each 5 mM), and the activity of the isolated mitochondria was evaluated. The results are shown in FIGS. 29A and 29B. As shown in FIGS. 29A and 29B, the mitochondria isolated in a solution having pH 8.0 to 8.9 decrease in its membrane potential as evidenced by the decreased fluorescent intensity in these samples.

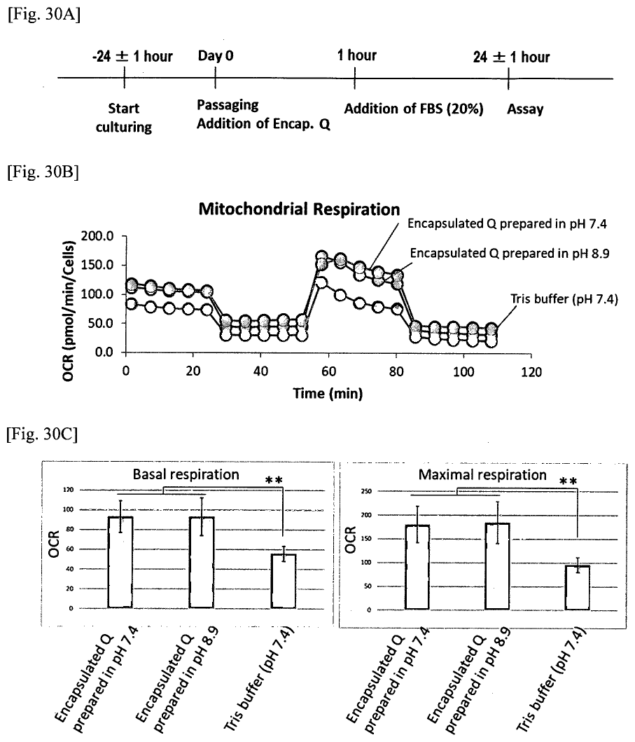

In order to evaluate the effect of transfer of mitochondria with decreased membrane potential on improving intracellular mitochondria function, the mitochondria isolated in a solution having pH 8.9, which have a decreased membrane potential, was encapsulated into a lipid membrane-based vesicle, and then was introduced into HeLa cells according to the procedures described in Example 9. The encapsulated mitochondria isolated in pH 7.4 solution were used as a positive control and the tris buffer was used as a negative control. Experiments were performed as shown in FIG. 30A. Briefly, HeLa cells were plated 24 before the experiment. 24 hours later, the cells were passaged and then the prepared encapsulated mitochondria were added to the cultures. After 1-hour incubation, FBS was added to the cultures at a final concentration of 20%. 24 hours later, cellular respiration function was measured as described in Example 9.

As shown in FIGS. 30B and 30C, mitochondrial respiration in the treated cells were improved in a group treated with encapsulated mitochondria prepared in a pH 8.9 buffer. This improvement of the respiration function was comparable to a group treated with encapsulated mitochondria prepared in a pH 7.4 buffer and was a significantly large improvement compared to the negative control group. From these results, it was considered that the membrane potential of mitochondria is not necessarily required to improve the mitochondria function within the cells into which the mitochondria were transferred.

### Example 12

mtDNA Abundancy in MITO-Q and its Impact on the MITO-Q Activity

MITO-Q was prepared from Q isolated in pH7.4 buffer, Q isolated in pH8.9 buffer, and mitochondria isolated by a conventional method using a detergent at a concentration higher than the critical micelle concentration (hereinafter also referred to as “D method”).

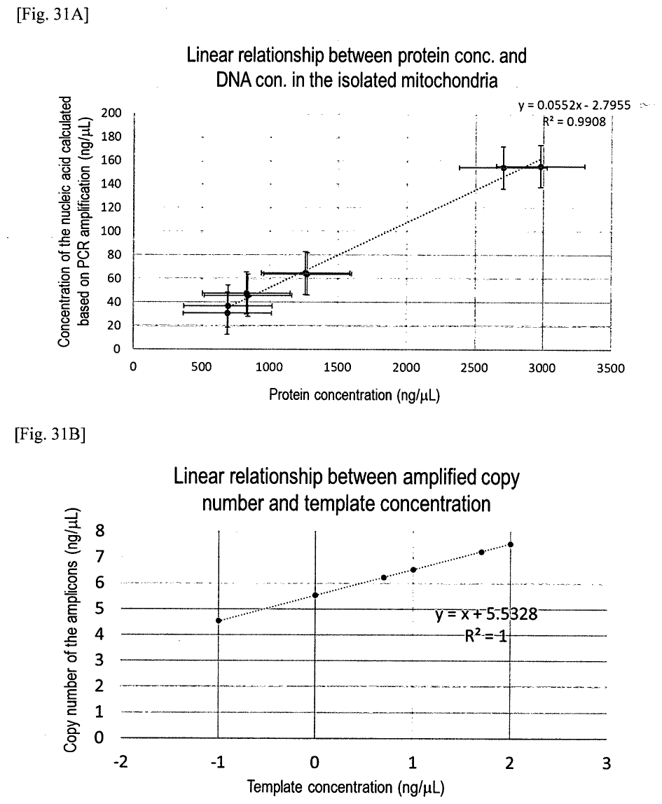

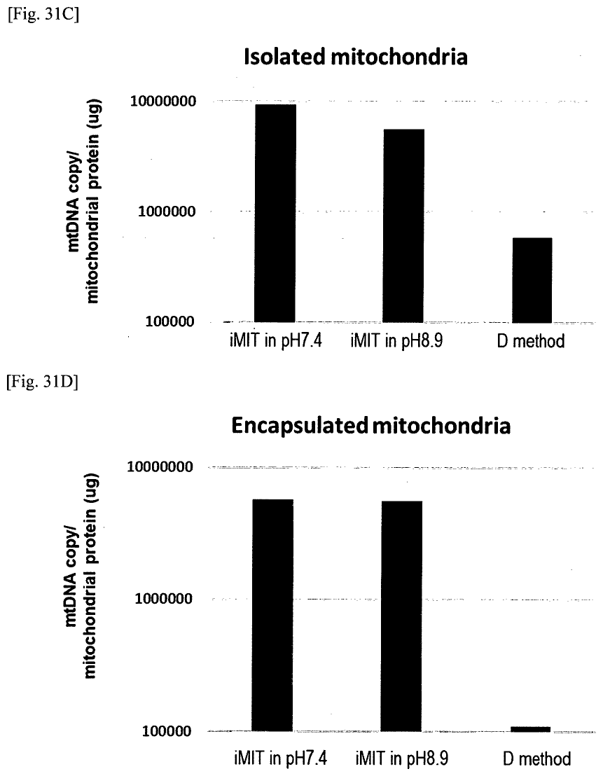

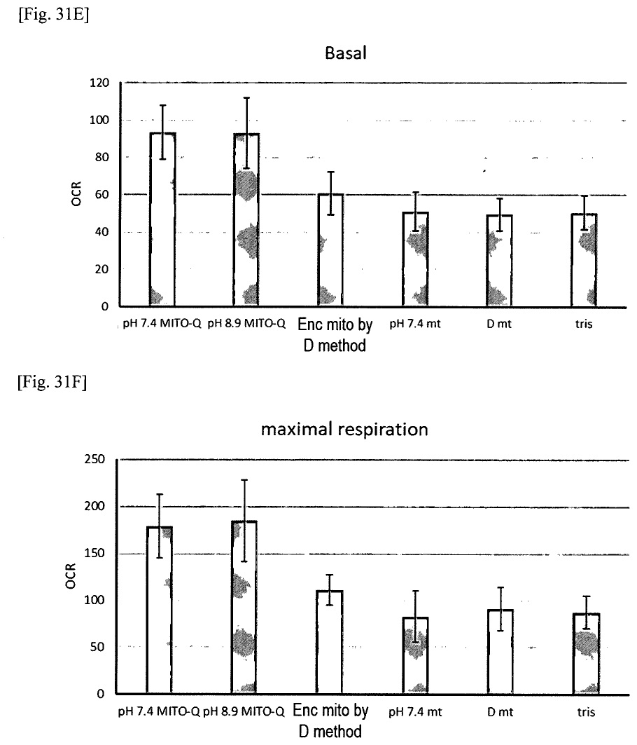

Mitochondrial DNA (also referred to as “mt DNA”) was measured by the quantitative PCR method. Before the measurement, a primer set was selected in order to detect mtDNA in a linear fashion for a wide dynamic range. After selecting a vast number of primer pairs, a forward primer having a nucleic acid sequence set forth in SEQ ID NO: 1 and a reverse primer having a nucleic acid sequence set forth in SEQ ID NO: 2 were identified to achieve a linear amplification of mtDNA by PCR. In FIG. 31A, the isolated mitochondria were diluted with Tris buffer to obtain a dilution series. Protein concentration was measured by a conventional Bradford method and the concentration of mtDNA was calculated by PCR amplification using the above-described primer pair. The relationship between the protein conc. and mtDNA conc was shown in FIG. 31A. As shown in FIG. 31A, the concentrations were related with each other in a highly linear manner. Further, pT7-tRNA^{Leu} was prepared by inserting tRNA^{Leu} (3230-3304) into EcoRI and EcoRV restriction enzyme sites of a pUC57-Amp plasmid vector. The plasmid was diluted with Tris buffer to obtain a dilution series. Then, the plasmid having a different concentration was amplified to measure a copy number of the amplicons. FIG. 31B shows that the copy number (or concentration of the amplicons) (ng/μL) measured was highly correlated with a concentration of plasmid, suggesting that the selected primer pair can linearly amplify mtDNA and is useful in quantifying the template mtDNA included in a sample.

If the membrane of the isolated mitochondria is broken, mtDNA will leak out from the mitochondria, resulting in the decrease in the amount of mtDNA in the obtained mitochondria. The number of mtDNA in the mitochondria isolated by the various method was calculated. The mitochondria isolated by iMIT in pH7.4 solutions have 9.3×10⁶ copy/μg protein of mtDNA, the mitochondria isolated by iMIT in pH8.9 solutions have 5.6×10⁶ copy/μg protein of mtDNA, and the mitochondria isolated by D method at a concentration higher than the CMC have 8.7×10⁵ copy/μg protein of mtDNA, provided that 1 ng of mtDNAs contain 10×10⁶ copy of mt DNA, as shown in FIG. 31C. Thus, it was thought the mitochondria isolated by iMIT keep their mtDNA inside the mitochondria, while the mitochondria isolated by D method lost most of their mtDNA from the mitochondria during the isolation process.

Further, these isolated mitochondria were packaged to obtain encapsulated mitochondria, and then, the copy of mtDNA in the encapsulated mitochondria was calculated in a similar manner. The results are shown in Table 1 and FIG. 31D.

TABLE 1
Copy number of mtDNA in the encapsulated
mitochondria differently obtained

| | iMIT | iMIT | |
|---|---|---|---|
| | (pH 7.4) | (pH 8.9) | D method |
| Recovery rate of | 28.7 | 37.9 | 41.7 |
| mitochondria protein (%) | | | |
| mtDNA/protein (copy/μg) | 5.7 × 10⁶ | 5.5 × 10⁶ | 1.2 × 10⁵ |

The obtained encapsulated mitochondria were contacted with cells to introduce the mitochondria into the cells. The basal mitochondria respiration and maximal mitochondria respiration were measured. The results are shown in FIGS. 31E and 31F. As shown in FIGS. 31E and 31F, MITO-Q isolated in pH 7.4 and MITO-Q obtained in pH 8.9 show the dramatic increase in the respiration activity, while the mitochondria obtained by D method shows the moderate increase in the respiration activity. From these results, mitochondria may lose some of the mitochondrial components during isolation process, while Q isolated by iMIT maintains them.

The amount of Transcription Factor A, Mitochondrial (TFAM) included in each of the encapsulated mitochondria was measured with Enzyme-linked Immunosorbent Assay Kit For Transcription Factor A, Mitochondrial (TFAM) organism Species: Homo sapiens (Human) (#MBS2706301) according to the manufacturer's manual. The mitochondria isolated by iMIT method using pH 7.4 buffer or pH 8.9 buffer, or by D method were used as the isolated mitochondria. These mitochondria were encapsulated into a lipid-membrane-based nano vesicle according to the Examples shown above. The results from the WST-1 assay are shown in FIG. 31G. As shown in FIG. 31G, the levels of TFAM included in the mitochondria isolated by the various methods were comparable to one another. As for the amount of total protein in the mitochondria isolated by these methods was comparable to one another (FIG. 31H).

The divided mitochondria or divided Q loses its respiration activity, and thus, some of the mitochondrial components that decrease during isolation process by the conventional method may play an important role in activating the mitochondrial function. It was also shown that the concentration of mtDNA in the vesicle will be an important indicator of the MITO-Q function.

### Example 13

Preparation of Lipid Membrane-Based Vesicle Comprising Mitochondria from Human Cardiac Progenitor Cells

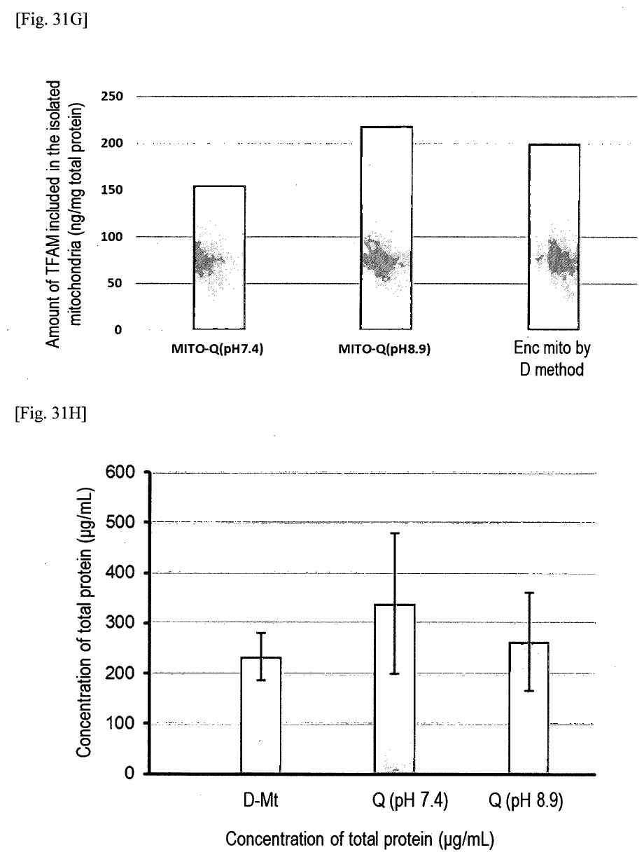

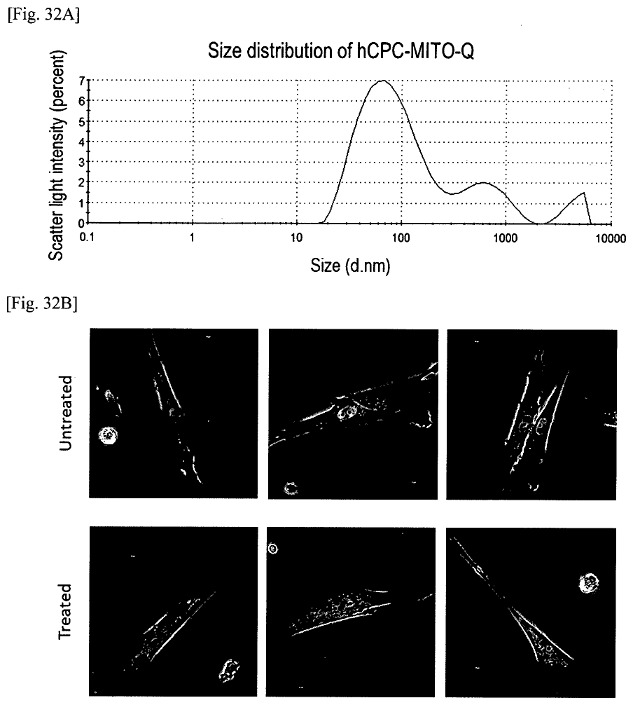

Mitochondria were isolated from human cardiac progenitor cells (hCPCs) according to Example 1, and then, were encapsulated into a lipid membrane-based vesicle according to Example 2. The size distribution of the isolated hCPC mitochondria and the encapsulated mitochondria were shown in FIG. 32A and Table 2 below.

TABLE 2
Average size, PDI and zeta potential of the isolated
hCPC mitochondria and the encapsulated mitochondria

| | Size (nm) | PdI | ζ potential (mV) |
|---|---|---|---|
| Isolated hCPC Mt | 805 | 0.70 | −34 |
| hCPC-MITO-Q | 85 | 0.44 | 43 |

As shown in Table 2 and FIG. 32A, the isolated hCPC mitochondria (i.e., isolated hCPC Mt) have a peak at nearly 800 nm in size distribution, PDI of 0.7, which is larger than 0.5, and the negative zeta potential. After encapsulated in a lipid membrane-based vesicle presenting cationic peptide, the vesicles (i.e., hCPC-MITO-Q) have a peak at 85 nm in size distribution, PDI of less than 0.5, and the positive zeta potential. These results indicate that the hCPC mitochondria were successfully encapsulated in a vesicle presenting a cationic peptide.

Then, the prepared encapsulated mitochondria (hCPC-MITO-Q) were contacted with hCPC to measure the degree of the membrane polarization of the resulting hCPC by staining the mitochondria using TMRM. As shown in FIG. 32B, hCPC-MITO-Q induces stronger fluorescence in the treated cells than in the untreated control cells.

### Example 14

Effect of the Transfer of the Isolated Mitochondria from Cells that are Activated in their Mitochondria Function by MITO-Porter

In this Example, hCPCs were treated with a mitochondria-activating agent Resveratrol by using MITO-Porter, which is a drug delivery system for mitochondria, in the same method as shown in Example 1 in WO2018/092839. Then, the mitochondria were isolated from the Resveratrol-treated hCPCs and encapsulated in a lipid membrane-based vesicle presenting S2 peptide (Szeto, H. H. et al., Pharm. Res., 2011, 28, pp. 2669-2679) according to the method described in Example 12, by using stealylated S2 peptide instead of stealylated octaarginine. The resulting vesicles were called Res-hCPC-MITO-Q.

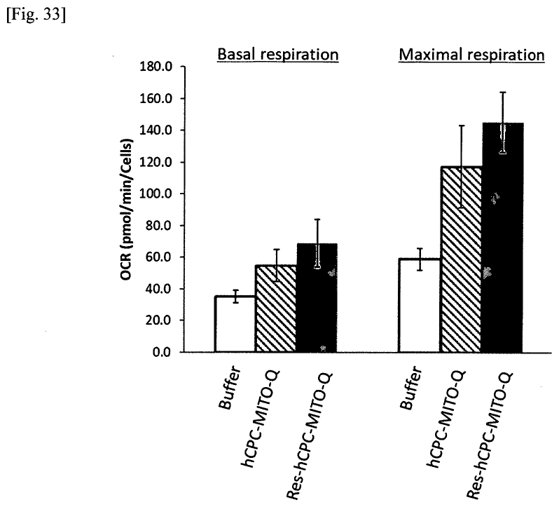

The basal respiration and maximal respiration of the hCPC treated with Tris buffer, hCPC-MITO-Q prepared in Example 12, and Res-hCPC-MITO-Q prepared in Example 13 were shown in FIG. 33. As shown in FIG. 33, Res-hCPC-MITO-Q induce a larger improvement in respiration capacity of the treated cells than hCPC-MITO-Q. These results indicate that activated mitochondria can further improve the respiration capability of the treated cells.

### Example 15

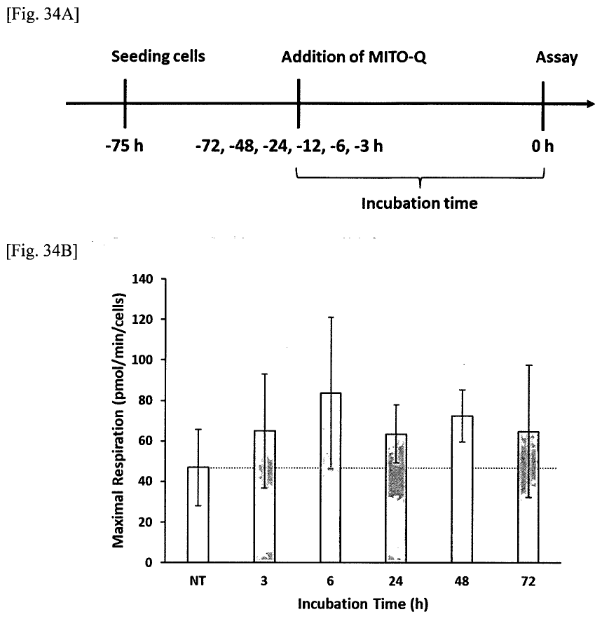

The MITO-Q prepared as shown in Examples 1 to 2 was contacted with normal fibroblasts and then the treated cells were incubated for a different time as indicated in FIG. 34A. The maximal respiration activity was measured in each sample. The results were shown in FIG. 34B. As shown in FIG. 34B, all of the treated cells show improved maximal respiration activities compared to a non-treated group (NT). It is thought that MITO-Q contains mitochondria DNAs and the other components of mitochondria, and that the improvement observed in a sample incubated for a shorter time (e.g., 3 hours to 48 hours) will be resulted from some of the non-DNA components including proteins, metabolites, and the like, while the improvement observed in a sample incubated for a longer time (e.g., 72 hours) will be resulted from mitochondria DNA transferred to the cells in view of the turnover period of the mitochondria (about 2 to 3 days).

### Example 16

### Storage of a Lipid-Membrane Based Vesicle Encapsulating Mitochondria

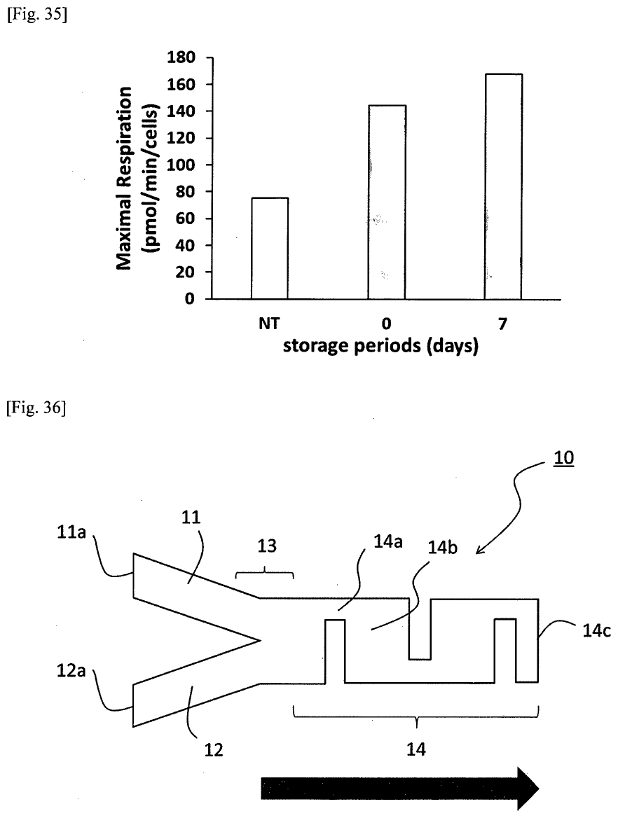

The mitochondria were isolated from hCPCs and encapsulated in a lipid membrane-based vesicle presenting S2 peptide/aptamer according to the method described in Example 12. The obtained hCPC-MITO-Q was stored at 4° C. for one week. Then, the stored hCPC-MITO-Q was contacted with hCPC to measure the maximal respiration capability. The hCPC-MITO-Q before the storage was used as a positive control. As shown in FIG. 35, the maximal respiration of the cells treated with the stored hCPC-MITO-Q was larger than the non-treated cells (NT) and the cells treated with the hCPC-MITO-Q before the storage. Thus, these results indicate that the formulation containing a lipid membrane based vesicle can be stably stored for at least one week.

### REFERENCE SIGNS LIST OF FIG. 36

11: Channel 1
11a: Liquid sample inlet of channel 1
12: Channel 2
12a: Liquid sample inlet of channel 2
13: Confluent channel of channel 1 and channel 2
14: Mixing channel for mixing liquid samples joined in confluent channel
14a: Narrowed region of channel
14b: Widened region of the narrowed region
14c: Outlet for discharging liquid sample mixture

What is claimed is:

1. A composition comprising a population of mitochondria, wherein the population has a particle size distribution, as determined by dynamic light scattering, having a peak at less than 1 μm.

2. The composition according to claim 1, wherein the population has a particle size distribution, as determined by dynamic light scattering, having a peak at less than 500 nm.

3. The composition according to claim 1 or 2, wherein the population has a PDI of less than 0.5.

4. A composition comprising a population of lipid membrane-based vesicles encapsulating mitochondria, wherein the population of lipid membrane-based vesicles has a particle size distribution, as determined by dynamic light scattering, having a peak at less than 1 μm.

5. The composition according to claim 4, wherein the population of lipid membrane-based vesicles has a particle size distribution, as determined by dynamic light scattering, having a peak at less than 500 nm.

6. The composition according to claim 4 or 5, wherein the population of lipid membrane-based vesicles has a PDI of less than 0.5.

7. The composition according to any one of claims 4 to 6, wherein the encapsulated mitochondria can be incorporated into the cytoplasm of cells in contact therewith, and the mitochondria can be fused with endogenous mitochondria in the cytoplasm.

8. The composition according to any one of claims 4 to 7, for use in delivering mitochondria into cells.

9. The composition according to claim 8, for use in improving respiratory activity of mitochondria in cells.

10. A method for producing the composition according to claim 4, comprising
bringing an aqueous solution comprising isolated mitochondria and an ethanol solution comprising a lipid that can form lipid membrane into contact with each other in a confluent channel within a micro flow channel device to mix the solutions.

11. The method according to claim 10, wherein the micro flow-channel device comprises a flow channel for facilitating mixing of the solutions brought into contact with each other in the confluent channel, the flow channel having a baffle construct.

12. A method of measuring mitochondria DNA level in the isolated mitochondria or the encapsulated mitochondria, comprising amplifying at least a part of the mitochondria DNA in a sample including the isolated mitochondria or the encapsulated mitochondria to obtain amplicon of the amplified DNA and counting the amplicon to obtain the mitochondria DNA level.

13. The method according to claim 12, further comprising comparing the measured mitochondria DNA level to a standard value.
